(19) United States
(12) Patent Application Publication
(10) Pub. No.: US 2005/0095594 A1
(43) Pub. Date: May 5, 2005

(54) Nucleic acid sequences of hyperplasies and tumors of the thyroid

(76) Inventors: Jorn Bullerdiek, Bremen (DE); Volkhard Rippe, Emtinghausen (DE); Maren Meiboom, Bremen (DE); Gazanfer Belge, Bremen (DE)

Correspondence Address: FOLEY & LARDNER LLP, 111 HUNTINGTON AVENUE, 26TH FLOOR, BOSTON, MA, 02199-7610, US

(21) Appl. No.: 10/684,042

(22) Filed: Oct. 10, 2003

Related U.S. Application Data
(63) Continuation of application No. PCT/EP02/04090, filed on Apr. 12, 2002.

(30) Foreign Application Priority Data
Apr. 12, 2001 (DE) 10118452.2

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

The invention relates to a nucleic acid that has a modified expression caused by hyperplasias and/or tumours, said nucleic acid comprising a nucleic acid sequence selected from the group including SEQ. ID. No. 1 to 12 and SEQ. ID. No. 16 to 19.

The present invention relates to nucleic acids that have a modified expression caused by hyperplasias and/or tumours, nucleic acids coding for the human homologues to CAT, DC2, hereinafter also called CAT and DC2, and PKCgamma and especially homologues thereof, containing vectors and cells containing these, polypeptides encoded by these, antibodies directed theretowards, methods for determining compounds suitable as therapeutic substances for tumours, methods for determining genes involved in the formation of tumours of the thyroid gland and uses of said nucleic acids.

The importance of the thyroid gland as a result of its hormone production for controlling the growth and development of the body has been known for a long time in medicine in the same way as the eye-catching changes caused by a functional disorder of this gland, at least in some cases, in the truest sense of the word.

With regard to the pathogenesis of goitres and tumours of the thyroid gland in general, it has not yet been possible to develop any comprehensive picture, especially on the molecular level. Two concepts are currently being discussed, of which one assumes that hyperplastic tissue is conditionally considered to be the result of chronic stimulation by a trophic hormone which ultimately results in the growth of polyclonal nodules. This concept is called non-neoplastic endrocrine hyperplasia (NNEH). The second concept assumes that the nodules are genuine clonal tumours.

In the case of the thyroid gland it has been found that the simple concept of non-neoplastic endrocrine hyperplasia which can be applied to other glands, cannot be applied. A review of the current thinking in this field is found in Studer, H. (1995); Endocrine Reviews, Vol. 16, No. 4, pp. 411-426.

Thus, an early diagnosis is required for the treatment of goitres and tumours of the thyroid so that suitable therapeutic concepts can be applied.

The object of the present invention is to provide means for the diagnosis and treatment of functional disorders of the thyroid, hyperplasias of the thyroid and tumours of the thyroid on the molecular level, especially to provide nucleic acid sequences which are involved in the pathogenicity mechanisms and are also suitable for investigating these mechanisms, as well as those which act on the pathogenicity mechanisms or can influence these mechanisms.

In addition, medicaments based thereon and general pharmaceutical compositions should also be provided.

Furthermore, kits for the diagnosis and/treatment of functional disorders of the thyroid, hyperplasias of the thyroid and tumours of the thyroid as well as methods for detecting these should also be provided.

The object is solved according to the invention by the subject matter of the independent claims. Further embodiments are obtained from the dependent claims.

The object is solved according to the invention in a first aspect by a nucleic acid that has a modified expression caused by hyperplasias and/or tumours, said nucleic acid comprising a nucleic acid sequence which is selected from the group comprising SEQ. ID. No. 1, 2, 3, 4, 5, 6, 7, 8, 9, 10, 11, 12, 16, 17, 18 and 19, hereinafter also called SEQ. ID. No. 1 to 12 and SEQ. ID. No. 16-19.

In one embodiment it is provided that the tumour is selected from the group comprising epithelial tumours with a change to the chromosomal band 19q and tumours with a change to the chromosomal band 19q13.

In another embodiment it is provided that the hyperplasia is selected from the group comprising hyperplasias of the thyroid.

In yet another embodiment it is provided that SEQ. ID. No. 1, 2, 7, 10, 11 and/or 12 codes for a CAT, especially a human CAT, or a part thereof.

In another embodiment it is provided that that SEQ. ID. No. 3, 4, 8, 16 and/or 17 codes for a DC2, especially a human DC2, or a part thereof.

In yet another embodiment it is provided that in that SEQ. ID. No. 5, 6, 9, 18 and/or 19 codes for PKCgamma, especially human PKCgamma, or a part thereof.

In another aspect, the object according to the invention is solved by a nucleic acid comprising a nucleic acid sequence which, without the degeneration of the genetic code, would code for the same amino acid sequence as a nucleic acid according to the invention.

In another aspect the object according to the invention is solved by a nucleic acid which hybridises to one or with one of the nucleic acids according to the invention.

In yet another aspect the object is solved by a vector, said vector comprising at least one of the nucleic acids according to the invention.

In one embodiment it is provided that the vector furthermore comprises at least one element selected from the group comprising promoters, terminators and enhancers.

In another embodiment it is provided that the vector is an expression vector.

In yet another embodiment it is provided that at least one promoter is in the reading frame with at least one part of a nucleic acid coding for a polypeptide according to any one of claims 1 to 8.

In another aspect the object according to the invention is solved by a polypeptide coded for by a nucleic acid according to the invention.

In one embodiment it is provided that the polypeptide is modified.

In another aspect the object according to the invention is solved by a cell, especially an isolated cell which comprises a vector according to the invention.

In another aspect the object according to the invention is solved by an antibody which is directed against a polypeptide according the invention.

In one embodiment it is provided that the antibody is directed against a nucleic acid according to the invention.

In another aspect the object according to the invention is solved by a ribozyme which is directed against a nucleic acid according to the invention.

In one embodiment it is provided that the ribozyme comprises at least a part of one of the nucleic acids according to the invention.

In another aspect the object according to the invention is solved by an antisense nucleic acid comprising a sequence complementary to or identical with one of the nucleic acids according to the invention.

In another aspect the object according to the invention is solved by RNAi comprising a sequence complementary to or identical with one of the nucleic acids according to the invention, wherein preferably the RNAi comprises a region having a length of 21 to 23 nucleotides which is complementary or identical.

In another aspect the object according to the invention is solved by a method for determining a compound which influences the effect of a translation product of a nucleic acid according to any one of the preceding claims, especially inhibits said effect, characterised by the following steps:
Preparation of the translation product and the compound
Bringing in contact the translation product and the compound in a system which represents the effect of the translation product, and
Determining whether any modification of the effect of the translation product occurs under the influence of the compound.
In yet another aspect the object according to the invention is solved by a method for determining a compound which influences the effect of a transcription product of a nucleic acid according to any one of the preceding claims, especially inhibits said effect, characterised by the following steps:
Preparation of the transcription product and the compound
Bringing in contact the transcription product and the compound in a system which represents the effect of the transcription product, and
Determining whether any modification of the effect of the transcription product occurs under the influence of the compound.

In an embodiment of the two aforesaid methods according to the invention it is provided that the system is selected from the group comprising cellular expression systems, cell-free expression systems, assay to determine the interaction between compound and translation products and assay to determine the interaction between compound and transcription products.

In another aspect the object according to the invention is solved by a method for determining genes responsible for the formation of hyperplasias and tumours, especially of the thyroid, comprising the following steps:
Determination of break points at chromosomal translocations of the hyperplasias and the tumours,
Determination of genes which lie within a region of 400 kbp, preferably 150 kbp, in each direction from the break point region, and
Determining whether the translation/transcription of the gene in a cell of the hyperplasia or of the tumour is modified compared with a non-hyperplasia cell or a non-tumour cell.

In yet another aspect the object according to the invention is solved by using one of the nucleic acids according to the invention and/or a ribozyme according to the invention and/or an antisense nucleic acid according to the invention and/or an RNAi according to the invention for the diagnosis and/or treatment of functional disorders of the thyroid and/or hyperplasias of the thyroid and/or thyroid tumours.

In yet another aspect the object according to the invention is solved by using one of the nucleic acids according to the invention and/or a ribozyme according to the invention and/or an antisense nucleic acid according to the invention and/or an RNAi according to the invention for the manufacture of a medicament, especially for the treatment and/or prevention of functional disorders of the thyroid and/or hyperplasias of the thyroid and/or thyroid tumours.

In another aspect the object according to the invention is solved by using a polypeptide according to the invention for the diagnosis and/or treatment of functional disorders of the thyroid and/or hyperplasias of the thyroid and/or thyroid tumours.

In another aspect the object according to the invention is solved by using a polypeptide according to the invention for the manufacture of a medicament, especially for the treatment and/or prevention of functional disorders of the thyroid and/or hyperplasias of the thyroid and/or thyroid tumours.

In another aspect the object according to the invention is solved by using an antibody according to the invention for the diagnosis and/or treatment of functional disorders of the thyroid and/or hyperplasias of the thyroid and/or thyroid tumours.

In another aspect the object according to the invention is solved by using an antibody according to the invention for the manufacture of a medicament.

In yet another aspect the object according to the invention is solved by a kit for the diagnosis of functional disorders of the thyroid and/or hyperplasias of the thyroid and/or thyroid tumours, characterised in that the kit comprises at least one element selected from the group comprising a nucleic acid, a vector, a polypeptide, a cell, an antibody, an antisense nucleic acid, RNAi and a ribozyme, each according to the present invention.

In another aspect the object according to the invention is solved by a method for detecting functional disorders of the thyroid and/or hyperplasias of the thyroid and/or thyroid tumours, characterised by the following steps:
contact of thyroid material with the agent selected from the group comprising a nucleic acid, a vector, a polypeptide, an antibody, an antisense nucleic acid, RNAi, a ribozyme and a cell, each according to the present invention, and
determining whether functional disorders of the thyroid and/or hyperplasias of the thyroid and/or thyroid tumours are present.

In one embodiment it is provided that the thyroid material is present ex vivo.

In another aspect the object according to the invention is solved by using a nucleic acid according to the invention as a primer and/or a probe.

In yet another aspect the object according to the invention is solved by a primer for representing and/or screening and/or detecting a nucleic acid, said primer being complementary to a part of one of the nucleic acids according the invention or identical therewith.

In another aspect the object according to the invention is solved by a method for representing a nucleic acid which comprises a sequence which can be detected in thyroid tumours or goitres, in which there is a translocation with a break point in the chromosomal band 19q13, wherein the sequence lies within the chromosomal band 19q13, characterised in that the method comprises the following steps:
Preparation of primers according to the invention for carrying out a polymerase chain reaction,
Preparing a nucleic acid sequence taken from the band 19q13 of the human chromosome 19 or one of the nucleic acids according to the invention according to on e of the . . .

Mixing the nucleic acid sequence or the nucleic acid with the primers,
Carrying out a polymerase chain reaction.

In another aspect the object according to the invention is solved by a pharmaceutical composition characterised in that it comprises:
at least one agent selected from the group comprising a nucleic acid, a vector, a polypeptide, a cell, an antibody, an antisense nucleic acid, RNAi, a ribozyme, each according to the present invention, as well as a combination thereof, and
at least one pharmaceutically acceptable carrier.

In yet another aspect the object according to the invention is solved by a method for the treatment and/or prevention of tumours and hyperplasias, wherein it is provided that a compound is administered to a patient which prevents or enhances the effects of modified expression of one of the nucleic acids according to the invention.

In another aspect the object according to the invention is solved by using a compound which prevents the effects of modified expression of the nucleic acids according to any one of the preceding claims, for the manufacture of a medicament.

In one embodiment it is provided that the medicament is for the treatment and/or prevention of tumours and/or hyperplasias, especially of tumours and/or hyperplasias of the thyroid.

In another aspect the object according to the invention is solved by using a nucleic acid having a sequence, wherein the sequence is selected from the group comprising SEQ. ID. No. 1-12 and SEQ. ID. No. 16-19, or derivatives thereof and/or polypeptides encoded thereby or derivatives thereof for the manufacture of a medicament, especially for the treatment of functional disorders of the thyroid and/or hyperplasias of the thyroid and/or thyroid tumours and/or for the manufacture of a diagnostic means, especially for the diagnosis of functional disorders of the thyroid and/or hyperplasias of the thyroid and/or thyroid tumours.

In one embodiment it is provided that the polypeptide has an amino acid sequence in accordance with SEQ. ID. No. 13, SEQ. ID. No. 14 and/or SEQ. ID. No. 15.

In another embodiment it is provided that the nucleic acid would hybridise with the nucleic acid according to one of the sequences SEQ. ID. No. 1-12 and/or SEQ. ID. No. 16-19 without the degeneracy of the genetic code.

In another aspect the object according to the invention is solved by using a polypeptide having a sequence, wherein the sequence is selected from the group comprising SEQ. ID. No. 13-15, or derivatives thereof for the manufacture of a medicament, especially for the treatment of functional disorders of the thyroid and/or hyperplasias of the thyroid and/or thyroid tumours and/or for the manufacture of a diagnostic means, especially for the diagnosis of functional disorders of the thyroid and/or hyperplasias of the thyroid and/or thyroid tumours.

In another aspect the object according to the invention is solved by a method for screening a means for the treatment of functional disorders of the thyroid and/or hyperplasias of the thyroid and/or thyroid tumours and/or a diagnostic means for the diagnosis of functional disorders of the thyroid and/or hyperplasias of the thyroid and/or thyroid tumours, comprising the steps:
a) Preparation of a candidate compound,
b) Preparation of an expression system and/or activity system;
c) Bringing the candidate compound in contact with the expression system and/or the activity system;
d) Determining whether under the influence of the candidate compound, the expression and/or the activity of a nucleic acid having a sequence, wherein the sequence is selected from the group comprising SEQ. ID. No. 1-12 and SEQ. ID. No. 16-19, or derivatives thereof and/or polypeptides encoded thereby and/or polypeptides having a sequence in accordance with SEQ. ID. No. 13-15 or derivatives thereof, is modified.

In one embodiment it is provided that the candidate compound is contained in a compound library.

In another embodiment it is provided that the candidate compound is selected from the group of compound classes comprising peptides, proteins, antibodies, anticalins, functional nucleic acids and small molecules.

In yet another embodiment it is provided that the functional nucleic acids are selected from the group comprising aptamers, aptazymes, ribozymes, spiegelmers, antisense oligonucleotides and RNAi.

In another aspect the object according to the invention is solved by using a nucleic acid having a sequence, wherein the sequence is selected from the group comprising SEQ. ID. No. 1-12 and SEQ. ID. No. 16-19, or derivatives thereof and/or polypeptides encoded thereby or derivatives thereof and/or a polypeptide having a sequence in accordance with SEQ. ID. No. 13-15 or a derivative thereof and/or an especially natural interaction partner of a nucleic acid having a sequence wherein the sequence is selected from the group comprising SEQ. ID. No. 1-12 and SEQ. ID. No. 16-19, or derivatives thereof and/or polypeptides encoded thereby or derivatives thereof and/or a nucleic acid coding therefor and/or an interaction partner of a polypeptide having a sequence in accordance with SEQ. ID. No. 13-15 or a derivative thereof as a target molecule for the development and/or manufacture of a diagnostic means for the diagnosis of functional disorders of the thyroid and/or hyperplasias of the thyroid and/or thyroid tumours and/or for the development and/or manufacture of a medicament for the prevention and/or treatment of functional disorders of the thyroid and/or hyperplasias of the thyroid and/or thyroid tumours.

In one embodiment it is provided that the medicament or the diagnostic means comprises an agent which is selected from the group comprising antibodies, peptides, anticalins, small molecules, antisense molecules, aptamers, spiegelmers and RNAi molecules.

In one embodiment it is provided that the agent interacts with a polypeptide having a sequence in accordance with SEQ. ID. No. 13-15 or a derivative thereof or an interaction partner thereof.

In an especially alternative embodiment it is provided that the agent interacts with a nucleic acid having a sequence, wherein the sequence is selected from the group comprising SEQ. ID. No. 1-12 and SEQ. ID. No. 16-19, or derivatives thereof and/or with a nucleic acid coding for an especially natural interaction partner, especially with mRNA, genomic nucleic acid or cDNA.

In another aspect the object according to the invention is solved by using a polypeptide that interacts with a peptide which is encoded by a nucleic acid having a sequence wherein the sequence is selected from the group comprising SEQ. ID. No. 1-12 and SEQ. ID. No. 16-19, or derivatives thereof and/or with a polypeptide in accordance with SEQ. ID. No. 13-15 and/or interacts with an especially natural interaction partner thereof for the development or manufacture of a diagnostic means for the diagnosis of functional disorders of the thyroid and/or hyperplasias of the thyroid and/or thyroid tumours and/or for the development or manufacture of a medicament for the prevention and/or treatment of functional disorders of the thyroid and/or hyperplasias of the thyroid and/or thyroid tumours.

In one embodiment it is provided that the polypeptide is selected from the group comprising antibodies and binding polypeptides.

In another aspect the object according to the invention is solved by using a nucleic acid that interacts with a polypeptide wherein the polypeptide is encoded by a nucleic acid having a sequence wherein the sequence is selected from the group comprising SEQ. ID. No. 1-12 and SEQ. ID. No. 16-19, or derivatives thereof and/or with a polypeptide in accordance with SEQ. ID. No. 13-15 and/or with an especially natural interaction partner thereof for the development or manufacture of a diagnostic means for the diagnosis of functional disorders of the thyroid and/or hyperplasias of the thyroid and/or thyroid tumours and/or for the development or manufacture of a medicament for the prevention and/or treatment of functional disorders of the thyroid and/or hyperplasias of the thyroid and/or thyroid tumours.

In one embodiment it is provided that the nucleic acid is selected from the group comprising aptamers and spiegelmers.

In another aspect the object according to the invention is solved by using a first nucleic acid that interacts with a second nucleic acid wherein the second nucleic acid has a sequence wherein the sequence is selected from the group comprising SEQ. ID. No. 1-12 and SEQ. ID. No. 16-19, or derivatives thereof and/or with a nucleic acid which codes for an interaction partner of a polypeptide having a sequence in accordance with SEQ. ID. No. 13, 14 or 15 for the development or manufacture of a medicament for the prevention and/or treatment of functional disorders of the thyroid and/or hyperplasias of the thyroid and/or thyroid tumours.

In one embodiment it is provided that the interacting first nucleic acid is an antisense oligonucleotide, a ribozyme and/or RNAi.

In another embodiment it is provided that the second nucleic acid is the respective cDNA or mRNA.

In another aspect the object according to the invention is solved by a pharmaceutical composition comprising at least one agent selected from the group as defined by at least one use according to the invention, and at least one pharmaceutically acceptable carrier, especially for the prevention and/or treatment of functional disorders of the thyroid and/or hyperplasias of the thyroid and/or thyroid tumours.

In another aspect the object according to the invention is solved by a kit for characterising the state of a thyroid gland or of tissue forming said gland or one or a plurality of cells forming said gland, comprising at least one agent that is defined by at least one of the uses according to the invention.

The present invention is based on the surprising finding that there are a series of genes or nucleic acid sequences whose modified expression compared with normal tissue can be suspected of being connected with the occurrence of tumours and hyperplasias and appears to be causally connected thereto.

Within the scope of the present invention it was established that in the case of certain hyperplasias or tumours, some change in the expression of certain genes or sequences occurs. Herein especially change should be understood as either an increase in the expression or, however, a decrease in the expression. The extent of the expression of the gene in question or the sequence in question in cells or tissue which is or are different from the cells of the hyperplasias or tumours is used as a reference here.

The tumours in question here include the first group of epithelial tumours, especially those with a change in the chromosomal band 19q, and the second group of tumours showing a change in the band 19q13. Herein especially change should be understood as a chromosome translocation, chromosome deletion, chromosome insertion and chromosome inversion.

The first group of tumours especially comprises tumours of the thyroid and preferably epithelial tumours of the thyroid. The second group of tumours comprises, among others, leukaemias (such as chronic lymphatic leukaemia, acute myeloid leukaemia), B-cell lymphoma, glioma, malignant fibrous histocytoma, osteosarcoma, leiomyosarcoma, liposarcoma, ovarian tumours, mammarian tumours, carcinoma of the kidney, carcinoma of the pancreas and gall bladder carcinoma.

According to the WHO classification, the tumours of the thyroid are mostly of epithelial origin and can be divided into benign and malignant forms. In the case of benign tumours of the thyroid a distinction is made between “genuine” adenomas and thyroid hyperplasias (benign, adenomatous goitre nodules) which are described as “tumour-like lesions”. These hyperplasias are mostly polyclonal nodules and frequently have a variable, macrofollicular structure with incomplete capsule formation. Thyroid adenomas on the other hand are encapsulated tumours which are derived from the follicular epithelium. These generally solitarily occurring tumours are uniformly constructed and differ structurally from the adjacent thyroid tissue. They can be classified microscopically (in terms of fine tissue) according to their degree of differentiation as normofollicular (simple/single), macrofollicular (colloid), microfollicular (foetal), or trabecular (embryonal) adenomas.

The abbreviation CAT used here stands for “cationic amino acid transporter” (transporter for cationic amino acids). This transporter is responsible for the transport of cationic amino acids and thus for supplying the cells with amino acids. As a result of the fact that some of the amino acids function as neurotransmitters, some of the transporters are also involved in signal transmission. At the present time, it cannot be eliminated that CAT-A possibly has a function different therefrom.

The homology established within the framework of the present invention is based on a comparison of the human sequences with CAT3 from Rattus norvegicus.

The known DC2 is a ubiquitary protein in cells which was first described in connection with Drosophila and there in connection with the early embryonic development. DC2 exhibits a protein kinase activity. In humans DC was described for the first time in connection with dendritic cells.

PKCgamma stands herein for Protein Kinase C gamma which mediates the phosphorylation of serine and threonine and in this way is involved in the transcription regulation. In this respect it already follows from this general function of Protein Kinase C gamma that this is a target which allows an intervention into the transcription event in the cell and thus also provides a starting point both for the diagnosis and also for the treatment of tumours and hyperplasias as described herein. The finding that this transcription-influencing factor is of central importance for the events in connection with the tumours described herein was surprising for the inventor.

It is within the scope of the present invention that the sequences according to the invention also comprise the respective complementary sequences.

Furthermore included within the scope of the present invention are those nucleic acid sequences which especially under the standard conditions for Northern Blot hybridisations for the detection of single-copy sequences hybridise with the sequences in accordance with SEQ. ID. No. 1 to SEQ. ID. No. 12.

The method according to the invention for determining a compound which can influence the effect of a translation product of one of the nucleic acids according to the invention can also involve a method for screening a compound library. Such compound libraries are preferably libraries of low-molecular compounds. Basically, the procedure is that the translation product of one of the nucleic acids according to the invention is brought in contact with a compound in a system, wherein the system represents the effect of said translation product and thus an effect of the compound on the translation product or reproduces this in the form a detectable signal. This effect can, for example, be expressed in a modified function with respect to strength, substrate specificity (recognition, binding, transport) and/or receptor specificity or affinity or membrane association, i.e., in the signal transmission. The effect could also have an influence on enzymatic processes in the cell. The effect can be examined (in vitro, in vivo and in situ) both in cell-free and in cellular systems, in the cell culture and in transgenic animal models.

Insofar as in the method or applications according to the invention reference is made to tumours or hyperplasias, all tumours or hyperplasias disclosed in connection with the nucleic acids according to the invention should be understood as comprised hereunder.

Thus new possibilities are opened up with the present nucleic acid sequences both for the diagnosis and for the treatment and for investigating the mechanisms associated with the occurrence of goitres and tumours of the thyroid or carcinomas of the thyroid. In particular, through a knowledge of the sequence it becomes possible to indirectly or directly use suitable diagnostic or therapeutic means at the molecular level in the sense of—pharmaceutical—composition and medicaments.

The nucleic acid sequences according to the invention can be used in a known fashion for the person skilled in the art for the formation of suitable means which can be used diagnostically and therapeutically in the above sense as well as kits and methods.

Nucleic acid should be understood as both DNA sequences and RNA sequences, including hybrids thereof, including derivatives derived herefrom with modified backbones such as PNA and LNA.

This also includes the fact that the nucleic acids are present as single-stranded, double-stranded or as a triple structure.

It can also be provided that the strandedness of the DNA, i.e., whether this is present as single-stranded or double-stranded for example, changes via the length of the nucleic acid sequence.

In particular, it can also be provided that the nucleic acid sequence according to the invention is not complete but is present as a fragment.

Furthermore, it is also within the scope of the disclosure presented herein that the nucleic acid sequences can be present in mutated form. Mutation should be understood herein as all mutations known to the person skilled in the art which can occur within a nucleic acid sequence, including point mutation as well as non-point mutations, i.e., inversions, insertions and deletions.

Especially under the aspect of the interaction of the nucleic acid according to the invention with other nucleic acids, the hybridisation criterion which is generally recognised in engineering should be used herein. It is recognised that by selecting suitable hybridisation conditions, the stringency of the hybridisation within a specific region can be modified and it thus becomes possible for the nucleic acid sequences according to the invention to hybridise to nucleic acid sequences whose degree of deviation from the incompletely corresponding, i.e., complementary sequence, can vary.

Nucleic acid sequence in the sense according to the invention should be understood as that sequence which would hybridise with one of the sequences according to the invention if it were not for the degeneration of the genetic code. This means that by viewing the open reading frame present in the nucleic acid sequence according to the invention or the open reading frames, this/these can be translated into an amino acid sequence using the genetic code. As a result of the degeneration of the genetic code, however, it is also possible, starting from an amino acid sequence obtained in such a fashion, again using the genetic code, to obtain a nucleic acid sequence which is so different that, taken for itself, it can possibly no longer hybridise with the nucleic acid sequence used to determine the amino acid sequence.

Starting from the nucleic acids disclosed herein, it is possible for the person skilled in the art to produce a suitable antisense nucleic acid, especially antisense RNA, which can interact with the nucleic acid sequences according to the invention. As a result of this interaction, it is possible to directly influence the processes involving the nucleic acid sequences. This interaction can take place, for example, on the transcription level in the same way as on the translation level.

Nucleic acid sequences should be understood herein as generally nucleic acid sequences which can be obtained by isolation from said tissue in situ or ex vivo, for example, from corresponding cell, tissue or organ cultures. However, corresponding nucleic acid sequences which can be isolated from gene banks, especially human gene banks and more preferably gene banks of the human chromosome 19, should also be understood herein. Furthermore, the term nucleic acid sequences should also herein comprise nucleic acids which can be produced by means of suitable synthesis techniques, including the polymerase chain reaction and other biochemical and chemical synthesis methods known in the prior art.

The sequences according to the invention can also be modified.

Modification should be understood herein among other things as fragmentation, insertion, deletion and reversion of (part) sequences of the nucleic acid sequences according to the invention. This also includes the insertion of other nucleic acid sequences. These nucleic acid sequences can, for example, code for specific domains, serve as spacers and as elements for regulation of translation and transcription.

Furthermore, the nucleic acids according to the invention can be modified such that they comprise sequences or molecules which allow interaction with other molecules. This can be accomplished, for example, in the form of a binding site to a solid carrier or a sequence which mediates the binding to a nucleic acid-binding protein.

Furthermore, the nucleic acid sequences according to the invention can be labelled. Labelling should herein basically be understood as both direct and indirect labelling. Labelling can be accomplished using labellings and labelling methods known in the prior art and includes radioactive, non-radioactive and fluorescence labelling. Non-radioactive labellings comprise, among others, the use of digoxygenin, avidin, streptavidin and biotin.

Vector should herein especially be understood as recombinant vectors as known in technology. Such vectors include, among others, viral vectors, such as for example, adenoviral or retroviral vectors and phage systems as well as plasmid vectors, including cosmid vectors, and artificial chromosomes which can be used in prokaryotic and eukaryotic systems.

In addition to the nucleic acid sequences according to the invention, the vectors according to the invention can comprise further elements known in the prior art. The respective elements such as for example, promoters, terminators and enhancers are selected according to the respective host cell system in a fashion known to the person skilled in the art. Especially considered here is the choice of a suitable eukaryotic promoter and an inducible promoter. In addition to said elements, it is also still possible that such vectors contain such elements which have the result that at least the nucleic acid sequence according to the invention or a part thereof is integrated into the genome of the host cell system.

It is also possible that at least one of said elements in the reading frame (“in-frame”) is combined with at least one open reading frame of the nucleic acid sequences according to the invention and it is quite especially advantageous if the transcription rate of the special open reading frame is controlled by means of an additionally introduced promoter wherein the promoter is then typically at a suitable distance and “in-frame” with the open reading frame.

It can furthermore be provided that an open reading frame of the nucleic acid sequences according to the invention, preferably under the afore-said conditions, has a signal sequence which allows a translocation of the gene product encoded by the open reading frame via a membrane and if necessary, as a consequence thereof, a further modification of the gene product. Such signal sequences include those for the transport of synthesised proteins to the endoplasmic reticulum, Golgi apparatus, to lyosomes, to organelles such as mitochondria and chloroplasts as well as to the cell core. The passage possible in such a fashion through different cellular compartments allows a post-translational modification and thus, if necessary, a further advantageous development of the gene product.

In addition to signal sequences, such a construct can also contain additional nucleic acid sequences which have the result that the gene product of an open reading frame of the nucleic acid sequences according to the invention forms a fusion product wherein the fused-on part can correspond to a domain of another protein and for example, is used to detect the gene product of the open reading frame of the sequences according to the invention, or the interaction with other molecules or structures in biological systems wherein the biological system is preferably the cell.

In addition to the aforesaid and the further advantages of the nucleic acid sequences according to the invention obtained from the description by the person skilled in the art, these are also deducible from said nucleic acid sequences or inherent to polypeptides encoded thereby. These polypeptides can, on the one hand, be derived directly from an open reading frame of the nucleic acid sequence according to the invention or they can be produced in a fashion known to the person skilled in the art by a vector according to the invention expressed in a host organism. In this case, the host organism is typically initially transformed with the vector according to the invention, the host organism multiplies and the polypeptide is obtained from the host organism or, in the event of secretion of the same into the medium, from this medium.

In addition to a direct use of the polypeptides according to the invention and the nucleic acids encoding said polypeptides to influence the—cellular—events in the thyroid tissue, these can also be used in the purification, for example using affinity chromatography, of other components involved in the cellular events or for the production of suitable antibodies which then, among other things, can be used for their part for therapeutic and/or diagnostic purposes. In this fashion, among other things interaction partners of the polypeptides according to the invention can thus be determined or isolated.

Depending on the particular requirements such as post-translational modification or the desired degree of purity and the like, when the polypeptides according to the invention are produced in a host organism, either a prokaryotic or a eukaryotic host organism can be advantageous.

The polypeptide according to the invention can be modified in a suitable fashion. Modification should be understood, among other things, as a fragmentation, especially shortening of the molecule. Modification in the sense used herein also includes the labelling of the polypeptide. Said labelling can be achieved by means of both high-molecular and low-molecular compounds and includes radioactive, non-radioactive and fluorescence labelling. Labelling can also, for example, be in the form or phosphorylation or glycosylation of the protein. Corresponding labelling methods or modification methods are known in technology (see, for example, Protein Methods, 2^{nd} ed., D. M. Bollag et al., Wiley Liss, Inc., New York, N.Y., 1996) and are taken up herein to their full extent.

Modification is also understood according to the invention as any form of post-translational modification, as known among persons skilled in the art, especially proteolytic processing of the polypeptide, attachment or detachment of residue at the N-terminus, acetylation of the N-terminus, myristoylation of the N-terminus, attachment of glycosyl phosphatidyl residues, farnesyl residues or geranylgeranyl residues to the C-terminus, N-glycosylation, O-glycosylation, attachment of glycosaminoglycan, hydroxylation, phosphorylation, ADP-ribosylation and formation of disulphide bridges.

It is also within the scope of the present invention that the polypeptide according to the invention is or has been produced from a polypeptide according to the invention by mutation(s), especially amino acid mutation(s). Amino acid mutation is understood both as a mutation in which an amino acid is exchanged for a similar amino acid in its side chain (conservative mutation), for example, I to L or D to E, as well as a mutation in which one amino acid is exchanged for another amino acid without this exchange having a disadvantageous effect on the function of the encoded polypeptide (silent mutation). The function of the encoded polypeptide can be checked by means of a suitable assay. Suitable functional assays for CAT, i.e., transporters for cationic amino acids, for DC2, i.e., for the corresponding kinase activity, and for PKCgamma, i.e., for the Protein Kinase Cgamma are known to the persons skilled in the art and are described, for example, for CAT in Closs E I et al. Biochemistry 36(21): 6462-8; Wu F et al. Amm J Physiol Regul Integr Comp Physiol 278(6): R1506-12; and Palacin M et al. Physiol Rev 78(4): 969-1054 or for PKCG these are described in Becton DL et al., J Cell Physiol 125(3): 485-91; Persons D A et al, Cell Growth Differ 2(1): 7-14 and Perander M et al., J. Biol. Chem 276(16): 13015-24.

Further advantages can be achieved with the cells according to the invention. These comprise, among other things, the production of corresponding nucleic acid sequences or gene products derived therefrom.

In particular, the insertion of the nucleic acid sequences according to the invention in the genome of a cell is of particular importance, for example, for the further study of the influence of such sequences, especially in the cellular environment. In this case, gene dose effects and the like can be investigated or used for diagnostic and/or therapeutic purposes. Quite especially advantageous appears to be a state in which, starting from diploid cells, only one chromosome contains one or a plurality of nucleic acid sequences and preferably inserted in the position corresponding to their position in the band 19q13 and the second chromosome exhibits no chromosome translation involving the chromosomal band 19q13. Such cells can be used, for example, as positive and/or negative controls in a diagnostic approach.

It is within the scope of the present invention that in addition to the respective translation product according to the invention and/or the nucleic acid(s) coding therefor, as herein described, other means can also be used to produce or also to suppress the effects arising from the respective translation product or the nucleic acid coding therefor. Such means can be determined within the scope of a so-called screening method. In this case, one or a plurality of candidate compounds are prepared in a first step. Candidate compounds in the sense of the present invention are such compounds whose suitability should be established in a test system to treat the diseases disclosed herein, especially functional disorders of the thyroid and/or hyperplasias of the thyroid and/or thyroid tumours or to be used as diagnostic means for diagnosis of the same. If the candidate compound shows a corresponding effect in the test system described, it represents a corresponding means, i.e., suitable in principle for the treatment of these diseases. In a second step the candidate compound is then brought in contact with a (translation product) expression system or with a (translation product) activity system. A translation product expression system is in this case an expression system which shows the expression of the translation product, the extent of the expression being fundamentally variable. A translation product activation system is in this case substantially an expression system, wherein the focus here is less on the expression of the translation product than on its activity or its activity state and its influenceability. In this connection it is notable that the translation product as such need not be the result of an actual expression process but can also be already added to the corresponding activity system as a polypeptide or protein. Specifically it is established in this case whether the activity of the translation product or the nucleic acid coding for it changes under the influence of the candidate compound. In this case, both a reduction in the expression or activity and an increase in the expression or activity can take place regardless of the presence of the respective specific expression system or activity system. The expression system and/or the activity system typically involves an in vitro attachment, such as for example a cell extract or a fragment of a cell extract such as a cell core extract for example. A translation product expression system in the sense of the present invention can however also be a cell, preferably a cell of the thyroid, the hyperplasias of the thyroid or the cells forming a thyroid tumour.

A determination as to the extent to which any increase or a decrease in the expression system takes place, can be made on every level of the expression, i.e., for example, by the increase or decrease in the quantity of nucleic acid coding for the translation product, especially the mRNA, or also of the translation product produced in the expression system under the influence of the candidate compound, i.e., the respective protein. The techniques required for this such as for example a method for quantifying mRNA are known to the person skilled in the art and are described for example in Sambrook et al. (Sambrook, Joseph: Molecular Cloning: a laboratory manual/J. Sambrook; E. F. Fritsch; T. Maniatis—Cold Spring Harbor, N.Y.: Cold Spring Harbor Laboratory Press, 19XX 1st edition under the title Maniatis, Thomas P.: Molecular Cloning; author of the 3rd edition: Joseph Sambrook and David W. Russell; bibliography ISBN 0-87969-309-6 ISBN 0-87969-576-5 ISBN 0-87969-577-3 1st-2nd edition, 4th [Dr.]-1989). Furthermore, methods for determining the content of the quantity of translation product are also known to the person skilled in the art, for example, by using suitable antibodies. Antibodies can be produced using generally known methods described for example in Current Protocols (“Current Protocols in Protein Science”, published by Coligan J. E.; Dunn B. M., Hidde L. P., Speicher D. W., Wingfield P. T.; Wiley & Sons). It is furthermore possible that within the scope of the expression system the translation product produce carries a label, wherein suitable labels are known to the person skilled in the art. Such a suitable labelling is for example labelling with His₆ (Janknecht R et al. (1991) Proc Natl Acad Sci USA 88 (20): 8972-6).

In the case of the translation product activity system the increase in the activity or decrease in the activity of the translation product is typically tested in a functional assay, as already described in connection with the definition of the mutated translation product, more precisely the mutated polypeptide according to the invention.

The bringing in contact of candidate compounds and translation product expression system or activity system is generally achieved by adding a preferably aqueous solution of the candidate compound to the corresponding reaction system i.e., to the expression system or the activity system which are herein also generally described as test systems. The aqueous solution can in this case preferably be a buffer solution.

Candidate compounds are generally used in the form that in every test, only one compound at a time is used in the corresponding test system. It is also within the scope of the present invention that corresponding tests are carried out in parallel and in a high throughput system. In the last step of the screening method according to the invention, consisting in determining whether the expression or activity of the translation product or a nucleic acid coding for it is changed under the influence of the candidate compound, the method is generally carried out by comparing the behaviour of the test system without adding the candidate compound with the behaviour of the test system with the addition of the candidate compound. The candidate compound is preferably contained in a compound library. Basically every compound library regardless of the compound class is feasible as a compound library. Suitable compound libraries are, for example, libraries of small molecules. However, it is also within the scope of the present invention that compound classes other than small molecules are used, such as, for example, peptides, proteins, antibodies, anticalins and functional nucleic acids.

It is also within the scope of the present invention that the translation product according to the invention or the nucleic acid coding for it can be used as target molecules for the production of the aforesaid compound classes such as especially peptides, proteins, antibodies, anticalins and functional nucleic acids. The corresponding compounds i.e. peptides, proteins, antibodies, anticalins and functional nucleic acids can then be used in the screening method according to the invention.

It is furthermore within the scope of the present invention that those compounds, i.e., peptides, proteins, antibodies, anticalins and functional nucleic acids which are produced against the translation product according to the invention or the nucleic acid(s) coding for it(them), are used as means in the sense of the present invention, i.e., as means for the treatment of functional disorders of the thyroid and/or hyperplasias of the thyroid and/or thyroid tumours or as corresponding diagnostic means.

Peptides and especially binding peptides in the sense of the present invention are preferably such proteins and peptides which bind to the translation product according to the invention or one or the interaction partner, especially the natural interaction partner(s) of the translation product according to the invention, preferably in biological systems, especially in the thyroid or hyperplasias of the thyroid or thyroid tumours and the cells forming them. The same also applies to the antibodies, anticalins, functional nucleic acids and small molecules described herein in connection with the use according to the invention. At the same time, every member of the aforesaid compound classes preferably interacts with the translation product according to the invention or a nucleic acid coding for it and thus influences the corresponding activity of the translation product or the nucleic acid coding for it.

However, it is also within the scope of the present invention to produce or use or subject to a corresponding screening method such members which interact with the interaction partner, especially the natural interaction partner of the translation product according to the invention or a nucleic acid coding for it and influence the expression or activity of the interaction partner or the nucleic acid coding for it. Here also the concept “influence” describes either an increase or a decrease in the expression, the scope of the expression or the activity as disclosed generally herein. As a result of the interaction of said members with the interaction partners of the translation product according to the invention or the nucleic acid coding for it, the cascade of reactions coupled to the translation product is interrupted, so that the naturally observed effect of the translation product according to the invention or the nucleic acid encoded thereby is interrupted which can be used within the scope of the therapy or diagnosis disclosed herein relating to functional disorders of the thyroid and/or hyperplasias of the thyroid and/or thyroid tumours.

These peptides, in the following also called herein binding peptides or binding proteins, can be screened or produced using methods known in the prior art such as for example phage display. The techniques are known to the persons skilled in the art. The typical procedure for the production of such peptides is thus that a peptide library is constructed, for example, in the form of phages, and this library is brought in contact with a target molecule, in the present case for example with a translation product or the natural interaction partner of the translation product. The binding peptides are then typically removed as a complex together with the target molecule from the non-binding members of the library. It is in this case within the scope of the knowledge of the persons skilled in the art that the binding properties depend at least to a certain extent on the specific test conditions in each case, such as for example salt content and the like. After separating the peptides binding to the target molecule with a higher or stronger affinity from the non-binding members of the library or the target molecule, in the present case from the translation product according to the invention, these can then be characterised. If necessary, an amplification step may be required before the characterisation, for example, by multiplication of the corresponding phages encoding the peptide or the peptides or protein(s). The characterisation preferably comprises the sequencing of the peptides binding to the translation product or its natural interaction partner according to which of the two molecules was used as the target molecule in the phage-display screening method. The peptides are basically not limited with respect to their length. Typically however, peptides having a length of 8-20 amino acids are contained or used in such methods. The size of the libraries is 10²-10¹⁸, preferably 10⁸-10¹⁵ different peptides. The anticalins represent a special form of peptides binding to target molecules, as are described, for example, in German Patent Application DE 197 42 706.

In the light of the technical teaching disclosed herein, antibodies can then also be produced against a gene product, especially a translation product according to the invention such as a polypeptide or the nucleic acid sequences according to the invention. Thus, in this case also the advantages known to the person skilled in the art from the presence of antibodies against chemical compounds are also obtained, especially for in vitro and in vivo applications. In particular, with these antibodies it is possible, for example, to purify the compounds according to the invention, i.e., the nucleic acids according to the invention and/or the polypeptides or translation products according to the invention, to detect them and also to influence the biological availability, including the biological availability, of the compounds to which the antibody is directed both in situ and ex vivo, in vivo and/or in vitro. More precisely, especially using monoclonal antibodies, the gene products can be specifically detected or on the cellular level an interaction of the gene products or nucleic acid sequence with other cellular components can be influenced and thus specific intervention in the cellular event can be achieved. Depending on the effect of the respective compounds to which the antibody is directed and on which its effect develops in the system under study, in principle stimulating and inhibiting effects can be achieved.

Antibodies are understood herein as both polyclonal antibodies and monoclonal antibodies. Especially preferred however are monoclonal antibodies as a result of the increased specificity. However, applications are also feasible in which the purity or specificity of polyclonal antibodies can be sufficiently used or the plurality of specificities and other properties achieved with polyclonal antibodies can be used in an advantageous fashion. The production and use of antibodies is described for example in Antibodies: A Laboratory Manual (E. Harlow & D. Lane, Cold Spring Harbor Laboratory, NY, 1988) and is not included herein.

Furthermore, it is within the scope of this invention that the antibody can also be a single-chain antibody.

It is also within the scope of the present invention that the antibody is fragmented, especially is shortened. This includes the fact that the antibody is fragmented, especially is shortened. This includes the fact that the antibody is largely truncated as long as the antibody-specific property, i.e., binding to a defined epitope is still given. Truncation is especially advantageous if the corresponding antibody is to be used on the cellular level since this has improved permeation and diffusion properties compared with a complete antibody.

Moreover, there are also provided other forms of modification known to the person skilled in the art and described, for example, in Antibodies: A Laboratory Manual (E. Harlow & D. Lane, Cold Spring Harbor Laboratory, NY, 1988). In general it can be stated that in principle, the modification of antibodies can be carried out in a similar fashion to that of polypeptides and to a certain extent, that of nucleic acids and the reasoning put forward on this above also applies correspondingly to the antibodies according to the invention.

Another class of compounds which can be used within the scope of the screening method according to the invention and for the production of medicaments and diagnostic means according to the invention are functional nucleic acids. Functional nucleic acids should be understood herein especially as aptamers, aptazymes, ribozymes, spiegelmers, antisense oligonucleotides and RNAi.

Aptamers are D-nucleic acids, either single-stranded or double-stranded, RNA- or DNA-based which specifically bind to a target molecule. The production of aptamers is described for example in European Patent EP 0 533 838. The following procedure is employed:

A mixture of nucleic acids, i.e., potential aptamers is prepared, wherein each nucleic acid consists of a segment of at least eight successive randomised nucleotides and this mixture is brought in contact with the target, thus in the present case with a translation product, especially a translation product according to the invention, nucleic acid(s) coding for it, interaction partners of the translation product, especially the natural interaction partners, and/or nucleic acid(s) coding for them, wherein nucleic acids which bind to the target, if necessary on the basis of an increased affinity compared with the affinity of the candidate mixture, are separated from the remainder of the candidate mixture and the nucleic acids binding to the target thus obtained are amplified. These steps are repeated many times so that at the end of the method, nucleic acids binding specifically to the respective target or target molecule, precisely the so-called aptamers, are obtained. It is within the scope of the present invention that these aptamers can be stabilised, for example, by introducing certain chemical groups known to the person skilled in the art of aptamer development. Aptamers are currently already used therapeutically. It is also within the scope of the present invention that the aptamers produced in such a fashion are used for target validation and as indicator substances for the development of medicaments, especially of small molecules.

A fundamentally similar principle forms the basis for the manufacture or production of spiegelmers which can be developed within the scope of the present invention on the basis of a translation product according to the invention, the nucleic acid(s) coding for it, the translation product interaction partner, especially the natural interaction partner and/or the nucleic acid(s) coding for it as the target molecule. The manufacture of spiegelmers is described for example in International Patent Application WO 98/08856. Spiegelmers are L-nucleic acids, i.e., consist of L-nucleotides and are substantially distinguished thereby that they have a very high stability in biological systems and at the same time, comparable with aptamers, can interact specifically with a target molecule or bind thereto. During the manufacture of spiegelmers the procedure is thus employed that a heterogeneous population of D-nucleic acids is produced, the population is brought in contact with the optical antipodes of the target molecule, in the present case thus, for example, with the D-enantiomer of the naturally occurring L-enantiomer of a translation product, then those D-nucleic acids which have not interacted with the optical antipodes of the target molecule are separated, the D-nucleic acids which have interacted with the optical antipodes of the target molecules are determined, if necessary separated and sequenced, and then L-nucleic acids whose sequence is identical to those sequence(s) determined previously for the D-nucleic acid(s) are synthesised. Similarly to the method for the manufacture of aptamers, by repeating the steps many times it is also possible here to enrich or produce suitable nucleic acids, i.e., spiegelmers, which bind the translation product, one or a plurality of its especially natural interaction partners according to which of the aforesaid compounds is used as target molecule, or a nucleic acid coding for it.

Another form of the functional nucleic acids which can be used according to the invention are so-called aptazymes. Aptazymes are described, for example, by Piganeau, N. et al. (2000), Angew. Chem. Int. Ed., 39, No. 29, Pages 4369-4373. A specific form of aptamers is involved here, which are distinguished by the fact that in addition to the aptamer fraction which binds specifically to the target molecule, in the present case a translation product or an interaction partner, it can also contain a ribozyme fraction with the consequence that after binding of the target molecule of the aptamer fraction of the aptazyme, the ribozyme fraction is activated and as a result, a nucleic acid functioning as the substrate of the ribozyme fraction of the aptazyme is cleaved. With a corresponding configuration of the ribozyme substrate, a change in the fluorescence can be observed for example as a result of a change in the spatial arrangement of a fluorescence donor relative to a fluorescence acceptor on the ribozyme substrate. Aptazymes are especially suitable in this respect for the application within the scope of target validation relating to a translation product and its interaction partner and as a diagnostic means in the sense of the present invention. Nevertheless, a therapeutic application within the scope as disclosed in connection with the ribozymes described herein is also possible.

A factor common to the aforesaid classes of compound is that they substantially bind to the respective protein, i.e., the or a translation product, preferably according to the invention or the interaction partner thereof but it is also within the scope of the present invention that these classes of compound and especially the functional nucleic acids as target molecule have as their subject the nucleic acid(s) coding for the aforesaid proteins or polypeptides.

Another class of compounds which can be produced or developed using a translation product, preferably a translation product according to the invention and/or an interaction partner thereof and especially the nucleic acid(s) coding for them are ribozymes, antisense oligonucleotides and RNAi.

A factor common to all these classes of compounds is that they do not develop their effect at the level of the translation product, i.e., on the level of the proteins (translation product and interaction partner thereof) but at the level of the nucleic acid(s) coding for the respective protein, especially the mRNA coding for a translation product or the mRNA coding for an interaction partner of the translation product. The genome DNA or the corresponding cDNA is also basically suitable as the target molecule.

Ribozymes comprise catalytically active nucleic acids which are preferably constructed of RNA and consist of two part regions.

The first part region is responsible for catalytic activity whereas the second part is responsible for a specific interaction with a target nucleic acid. If an interaction develops between the target nucleic acid and the second part of the ribozyme, typically by hybridisation of base regions substantially complementary one to the other, the catalytic part of the ribozyme can hydrolyse the target nucleic acid either intramolecularly or intermolecularly, the latter being preferred, for the case that the catalytic effect of the ribozyme is a phosphodiesterase activity. As a result, an, if necessary further, degradation of the encoding nucleic acid takes place, wherein the titre of the target molecule can be reduced both at the nucleic acid and at the protein level both intra-cellularly and extra-cellularly and thus a therapeutic approach for the treatment of functional disorders of the thyroid and/or hyperplasias of the thyroid and/or thyroid tumours is provided. Ribozymes whose use and construction principles are known to the persons skilled in the art are described for example in Doherty and Doudna (Ribozyme structures and mechanisms. Annu Rev Biophys Biomol Struct 2001; 30: 457-75) and Lewin and Hauswirth (Ribozyme gene therapy: applications for molecular medicine. Trends Mod Med 2001, 7:221-8).

With a view to a pharmaceutical composition comprising a ribozyme in addition to the pharmaceutically acceptable carrier or the use of the ribozyme according to the invention as a medicament, and especially for the treatment of functional disorders, hyperplasias and tumours of the thyroid, it is also possible for the ribozyme to be constructed such that it acts specifically on one or a plurality of nucleic acid sequences according to the invention and thus also controls the expression or translation on the cellular level, which is especially important from the therapeutic, but also from the diagnostic aspect. As a result of the fact that ribozymes show both intra- and intermolecular catalytic effects, it can also be provided that the nucleic acid sequences according to the invention are modified such that regions of these sequences themselves are cleaved by the ribozyme activity of the modified region. A particular therapeutic possibility also opens up herein whose development is possible for the person skilled in the art in the light of the sequence information now available.

Like the nucleic acid sequences according to the invention, ribozymes themselves are then especially advantageous but not only when they can be incorporated into the effector cell for example, by means of gene therapy. However, it is also feasible that corresponding modifications are made ex vivo and such modified cells are then available for re-implantation, either allogenous or autogenous. The production and use of ribozymes is disclosed in Ribozyme Protocols (Phillip C. Turner, Ed., Humana Press, Totowa, N.Y., 1997) and is included herein.

The use of antisense oligonucleotides for the production of a medicament or diagnostic means in the sense of the present invention is based on a basically similar mechanism of action. Antisense oligonucleotides hybridise as a result of base complementarity typically with a target RNA, normally with mRNA and thereby activate RNaseH. RNaseH is activated both by phosphodiester and by phosphorothioate-coupled DNA. However, phosphorodiester-coupled DNA is rapidly degraded by cellular nucleases with the exception of phosphorothioate-coupled DNA. These resistant, non-naturally occurring DNA derivatives do not inhibit RNaseH when they are hybridised with RNA. In other words, antisense polynucleotides are only effective as a DNA-RNA hybrid complex. Examples for such antisense oligonucleotides are found, among others, in U.S. patents U.S. Pat. No. 5,849,902 or U.S. Pat. No. 5,989,912. In principle, the essential concept of the antisense oligonucleotide involves providing a complementary nucleic acid against certain RNA. In other words, starting from the knowledge of the nucleic acid sequence of a translation product or its interaction partner(s), especially the respective mRNA, by means of base complementarity it is possible to produce suitable antisense oligonucleotides which lead to degradation of the encoding nucleic acid, especially the mRNA. This degradation can then be recorded qualitatively or quantitatively in an expression or activity system.

Another class of compounds which can be used, identified or manufactured as a medicament or diagnostic means in the sense of the present invention is the so-called RNAi. RNAi is a double-stranded RNA which mediates RNA interference and typically has a length of around 21 to 23 nucleotides. In this case, one of the two RNA strands corresponds to a sequence of one or the gene to be degraded. In other words, starting from the knowledge of the nucleic acid(s) coding for a translation product and/or its interaction partner, especially the mRNA, a double-stranded RNA can be produced, wherein one of the two RNA strands is complementary to said nucleic acid(s) coding for the translation product and/or its interaction partner, preferably to mRNA, and this then leads to degradation of the corresponding encoding nucleic acid and with this, to a reduction in the titre of the respective translation products, i.e., the proteins. The production and use of RNAi as a medicament or diagnostic means is described for example in the International Patent Applications WO 00/44895 and WO 01/75164.

With a view to the mechanisms of action of the classes of compounds described previously, namely ribozymes, antisense oligonucleotides and RNAi, it is thus within the scope of the present invention to use, in addition to the translation product according to the invention, i.e., the respective polypeptide or protein and its especially natural interaction partner(s), the nucleic acid(s) respectively coding for them, especially the corresponding mRNA, cDNA or genome DNA, for the manufacture of a medicament for the treatment of functional disorders of the thyroid and/or hyperplasias of the thyroid and/or thyroid tumours or for the manufacture of a diagnostic means for the diagnosis of the aforesaid diseases or functional disorders and for monitoring the course of the disease or the therapy used, either directly or as a target molecule.

When using the translation product according to the invention, the nucleic acid coding for it, the translation product interaction partner(s), especially the natural interaction partner(s) and/or the nucleic acid(s) coding for it/them as a target molecule for the manufacture or development of a medicament for the treatment or therapy of functional disorders of the thyroid and/or hyperplasias of the thyroid and/or thyroid tumours and also for the manufacture and/or development of means for the diagnosis of the same, libraries of small molecules can also be used. Also in this case the target molecule is brought in contact with a library and those members of the library which bind thereto are determined, if necessary separated from the other members of the library or from the target molecule and optionally further characterised. Here also the characterisation of the small molecule is made using methods known to the persons skilled in the art, so that, for example, the compound is identified and the molecular structure determined. The libraries comprise as few as two and up to as many as a few hundred thousand members.

In connection with the various classes of compounds disclosed herein which can be used as therapeutic means or diagnostic means according to the invention, there is also an aspect that certain classes interact directly with a translation product or its interaction partner or bind to this. However, it is also within the scope of the present invention that said compounds of the various classes, especially if peptides, antibodies, anticalins, aptamers, aptazymes and spiegelmers are involved, block the interaction partner of the translation product by a more or less specific interaction for the translation product. In this respect, the concept of the use of a translation product should also be understood herein to comprise the use of one or a plurality of the translation product interaction partner(s), such as for example receptors and transcription and translation factors. The methods described herein should then be modified insofar as instead of the translation product protein or the nucleic acid coding for it, an interaction partner thereof is then used in the various selection methods, assays, screening methods or manufacturing methods. Interaction partners of PKCG are, among others, compounds having phospholipids and phosphatidylserine residue, the binding in the latter case being independent of phospholipids but dependent on Ca 2+ (Pawelcyk T, Kowara, R. Matecki, A; Mol. Cell Biochem. 2000 June; 209(1-2): 66-77). Another binding partner of PKCG is phorboldibutyrate (PDBu)/diacylglycerol and lipid binding domains (CaLB) (Quest A F, Bell, R M; J. Biol. Chem. 1994 Aug 5: 269 (31): 20000-12). Furthermore PKCG exhibits an interaction and thus a form of binding with PH domains of Tec family protein tyrosine kinases Ttk and Emt (similar to ltk and Tsk); in this case, an interaction with PKC genes takes place (Pawelczyk T, Matecki A, Dettlaff A; FEBS Lett 1998 Feb. 13; 423(1): 31-4. Interaction partners of CAT-A or CAT-A1 are, among others, generally amino acids and viruses.

Within the scope of the present invention, interaction partners of a translation product, especially of a translation product according to the invention, especially natural interaction partners are described as such molecules and structures with which said translation product interacts. Especially included here are those molecules and structures with which the protein interacts in a biological system under normal and/or under pathological conditions.

With respect to the organisation of the kit for diagnosis and/or treatment of functional disorders, hyperplasia and tumours of the thyroid, it should be noted that the precise organisation of such a kit, i.e., which component(s) is or are explicitly included therein, is known to the person skilled in the art and especially are possible in the light of the reasoning put forward above regarding the effect or usability of the nucleic acid sequences according to the invention disclosed herein and their translation product. The kit according to the invention will at least comprise one of the elements according to the invention which is selected from the group comprising the nucleic acid(s) according to the invention, the vector, the polypeptide, the cell, the antibody, the ribozyme and the various compounds of the various classes, as were characterised herein, especially the antibodies, peptides, proteins, anticalins, small molecules, aptamers, spiegelmers, ribozymes, aptazymes, antisense oligonucleotides as well as RNAi, specific for the nucleic acids according to the invention or translation products according to the invention and the interaction partners thereof, especially the natural interaction partners as well as the nucleic acids coding for each of these, each preferably in their definition according to the invention. Further elements of the kit according to the invention are selected from the group comprising buffer, negative controls, positive controls and instructions for use. Typically the individual compounds or constituents are contained in the kit in dry or liquid form, preferably portioned for a single case of application. The kit can be used to determine functional disorders of the thyroid and/or hyperplasias of the thyroid and/or thyroid tumours. With regard to the use of the cells according to the invention, these can especially be used as reimplants or as positive or negative controls in corresponding diagnostic and therapeutic attempts.

In the method according to the invention for the detection of functional disorders and hyperplasias and/or tumours of the thyroid, also in the sense of carcinomas and goitres, it is possible for the thyroid material to be present in situ, ex vivo, in vivo or in vitro. The concept “thyroid material”, as used here, especially describes material, preferably cellular material of the thyroid in its normal and/or pathogenic state. The concept of thyroid material thus also comprises material from thyroids with a functional disorder, from hyperplasias of the thyroid, from tumours of the thyroid, including carcinomas and goitres. The determination as to whether a functional disorder, hyperplasia and/or tumour is present, is ultimately made by comparing the effect of the agent or agents used according to the invention on the thyroid material to be studied with its effect on “normal” thyroid tissue. Further embodiments of the method according to the invention are obtained for the person skilled in the art on the basis of the disclosure contained herein.

The reasoning put forward above with regard to the various embodiments and the advantages attainable therewith also applies correspondingly to the medicaments according to the invention and especially to those means for the diagnosis and/or treatment of functional disorders, hyperplasias and/or tumours of the thyroid in the form of the sequence(s) according to the invention or their use, in the form of the vector according to the invention or its use, in the form of the polypeptide according to the invention, or its use, in the form of the cell according to the invention or its use, in the form of the ribozyme according to the invention or its use which are disclosed therein. The same also applies to the compounds produced according to the invention or selected from the group comprising binding peptides, anticalins and functional nucleic acids, wherein a factor common to these compounds is that they are directed against the nucleic acid sequences according to the invention or polypeptides encoded thereby or their interaction partner(s) or the nucleic acids coding for them, i.e., they are manufactured or selected against these as target molecule. The corresponding medicaments can also have a content of one or a plurality of said agents or compounds.

The same also applies to the pharmaceutical compositions according to the invention. In this case it is possible for a pharmaceutical composition to comprise only one of the cited compounds in addition to the pharmaceutically acceptable carrier. Alternatively it is also within the scope of the invention that the pharmaceutical composition has a plurality of said compounds in addition to the pharmaceutically acceptable carrier.

Pharmaceutically acceptable carrier should be understood herein as all carriers known to the person skilled in the art which are typically selected depending on the form of application. A pharmaceutically acceptable carrier can, among other things, be water, buffer solutions, alcohol solutions and the like.

With regard to the method for the manufacture of primers according to the invention, it should be noted that they comprise those which allow a specific representation of the nucleic acid sequences according to the invention or parts thereof.

In the method for the representation of a nucleic acid sequence according to the invention, from whatsoever source, the nucleic acid sequences according to the invention or sequences corresponding to these using primers according to the invention as primers for a polymerase chain reaction, can be used in all their different designs. The design of suitable primers as well as the implementation of polymerase chain reactions is disclosed in PCR & PCR 2: A practical approach (M. J. McPherson, P. Quirke and G. R. Taylor, Eds., IRL Press, Oxford, England 1991, & M. J. McPherson and B. D. Hames, Eds., IRL Press, Oxford, England 1995) and is included herein.

The nucleic acids according to the invention, including nucleic acid sequences according to the invention, are on the one hand the homologue to the human CAT genes disclosed herein, that is also designated as CAT or as CAT-A or CAT-A1 in special forms of definition of the CAT homologue according to the invention, homologues to the human DC2 gene and the PKCgamma gene whose N-terminus is known.

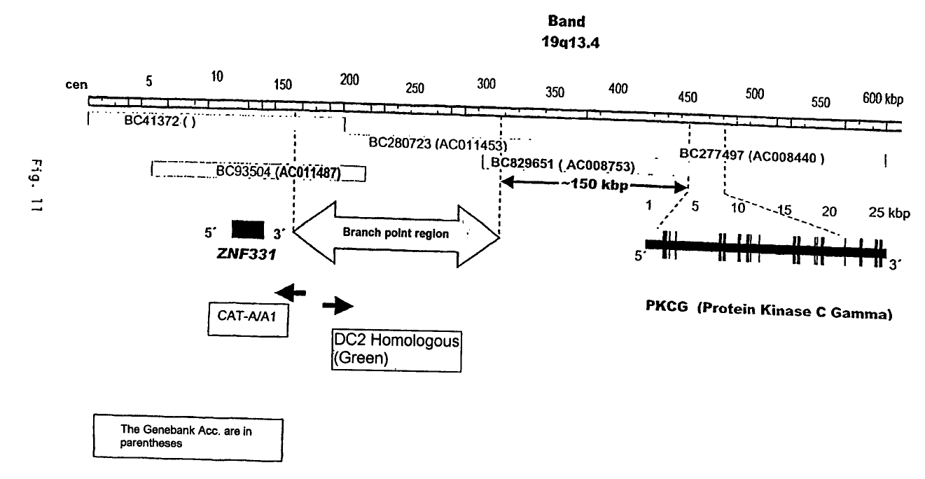

Nucleic acids or nucleic acid sequences according to the invention are especially also the nucleic acids cited hereinafter which comprise the nucleic acid sequences designated herein by “SEQ. ID. No.”, those nucleic acid sequences which partly or completely comprise the nucleic acid sequences designated herein by “SEQ. ID. No.” and comprise further nucleotides, especially at the 5′ and/or 3′ end, those nucleotides which contain further nucleotides in addition to the sequences designated herein by “SEQ. ID. No.”, in the case of the CAT-A or CAT-A1 sequence or gene according to the invention supplemented by further nucleotides from BC 93504, in the case of the PKCG gene supplemented by further nucleotides from BC 277497 or BC 829651 and in the case of DC2, i.e., the DC2 homologue according to the invention, supplemented by further nucleotides from BC 280723 or BC 93504. The relative arrangement of the aforesaid clones is shown in FIG. 11.

Furthermore, the nucleic acids according to the invention are those nucleic acids which code for a polypeptide, the polypeptide having or comprising a sequence as designated herein by one or a plurality of “SEQ. ID. No.”. Finally, nucleic acids according to the invention are also those which comprise one or a plurality of exons and/or one or a plurality of introns of one or a plurality of nucleic acids according to the invention disclosed herein.

The polypeptides according to the invention, which are also herein called polypeptides for short, are especially translation products of one or a plurality of the nucleic acids according to the invention. The translation products can be complete or shortened. These translation products are herein either generally described as translation product, regardless of the number, or as translation product according to the invention. Translation products according to the invention or polypeptides in the sense of the present invention are especially those parts of the corresponding polypeptide which are part of a domain structure, especially of such a domain which is present extracellularly in a cellular system.

The . . . for the nucleic acids according to the invention, especially for the CAT homologues according to the invention and DC2 homologues which are not known as such in the prior art, as well as PKCgamma are characterised or represented by the following nucleic acid sequences: The genome sequence from 19q13.4, beginning at cosmid 15841, continuing via BAC 280723, ends with the last bp of BAC 829651. The sequences are published with the accession numbers AC011453 (BAC280723), AC008753 (BAC 829651). The length of this genome sequence is 293395 base pairs. The nucleic acid sequence coding for the C-terminus for PKCgamma can be characterised or represented as follows: the genome sequence originates from 19q13.4 and is described in the sequence deposited under the accession number AC008440. This lies below or overlapping with BAC 829651. The length of the nucleic acid is 243085 base pairs. The nucleic acid sequence coding for the C-terminus of PKCgamma can furthermore be characterised by the following sequences: the genome sequence originates from 19q13.4 and is deposited with the accession number AC022318 and lies below or overlapping with the sequence BAC 829651. The total length of the sequence is 191422 base pairs:

The sequences disclosed here especially comprise human sequences wherein:
SEQ. ID. No. 1: corresponds to an EST (Acc No. BE 564764) of the CAT A or CAT-A1
SEQ. ID. No. 2: corresponds to an EST (Acc No. BF 029168) of the CAT-A or CAT-A1,
SEQ. ID. No. 3: corresponds to an EST (Acc No. AF201937) of the human DC2 gene,
SEQ. ID. No. 4: corresponds to an EST (Acc No. BE 271491) of the human DC2 gene,
SEQ. ID. No. 5: corresponds to an EST (Acc No. AF 345987) of the human PKCG gene and
SEQ. ID. No. 6: corresponds to an EST (Acc. No. X 625336) of the human PKCG gene, and
SEQ. ID. No. 7: designates the cDNA sequence of the CAT homologous gene on 19q13.4, length 647 bp,
SEQ. ID. No. 8: designates the cDNA sequence of the DC2 homologous gene on 19q13.4 (sequence according to the invention), length 749 bp; and
SEQ. ID. No. 9: designates the cDNA sequence of the PKCgamma homologous gene on 19q13.4, length 3457 bp, almost 100% homology as far as comparable, composed of Acc. No. X625337 and AF345987, and
SEQ. ID. No. 10 designates the cDNA of the CAT homologous gene disclosed herein as CAT-A,
SEQ. ID. No. 11: designates the cDNA sequence of a splice variant of CAT-A, designated herein as CAT-A1,
SEQ. ID. No. 12: designates the genome sequence of CAT-A or CAT-A1
SEQ. ID. No. 13: designates the amino acid sequence of CAT-A reading frame (ORFstart1),
SEQ. ID. No. 14: designates the amino acid sequence of CAT-A reading frame (ORFstart2),
SEQ. ID. No. 15: designates the amino acid sequence of CAT-A reading frame (ORF3),
SEQ. ID. No. 16: designates the genome sequence of the DC2 gene according to the invention which is homologous to the DC2 gene known in the prior art, consequently represents another form of the family of the DC2 gene;
SEQ. ID. No. 17: is the cDNA sequence of the DC2 homologous gene or the sequence in accordance with SEQ. ID. No. 16;
SEQ. ID. No. 18: is the genome sequence of PKCG; and
SEQ. ID. No. 19: is the cDNA of PKCG, consequently of SEQ. ID. No. 18.

It is within the scope of the present invention that every use disclosed herein for one or a plurality of polypeptides according to the invention or of the compounds derived herefrom as disclosed herein, such as small molecules, binding peptides, antibodies, anticalins and functional nucleic acids is basically also hereby disclosed for each of the other aforesaid molecules or compounds.

The invention is explained below with reference to the drawings and examples as well as the sequence protocol from which further features, embodiments and advantages of the invention are obtained. In the figures:

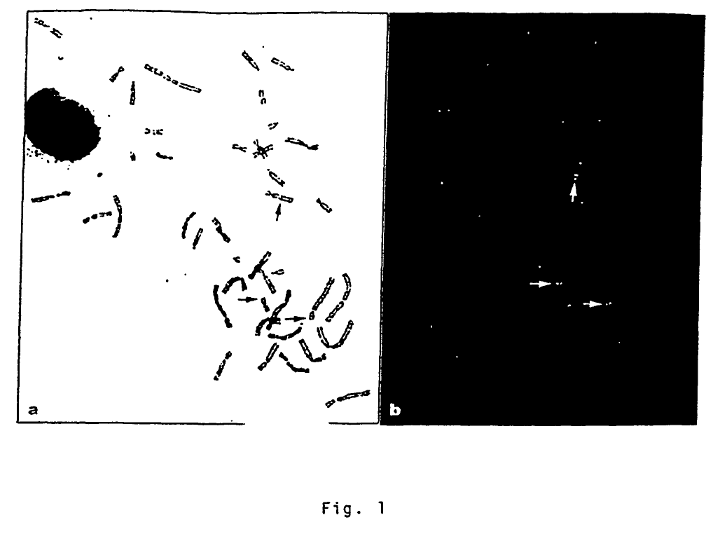

FIG. 1 is a metaphase of the cell line S 141.2 with t(2;19)(p12;q13) after G-banding wherein the chromosomes 19, der(19) and der(2) are marked with an arrow (a); FIG. 1(b) is the same metaphase after FISH with the BAC clone 280723 (the hybridisation signals are localised on the chromosomes 19, der(19) and der(2).

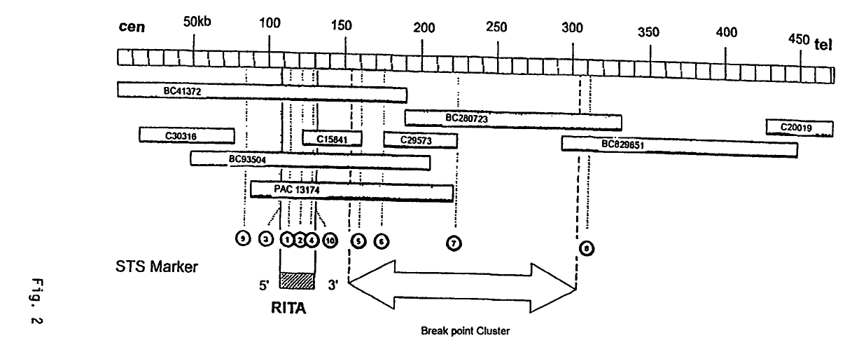

FIG. 2 is a schematic diagram of the break point cluster region in benign thyroid tumours with 19q13 aberrations. The FISH mapping data of the six tumours tested in the trial show that the break points of the benign thyroid tumours with 19q13 translocations in the region around 150 kbp 3′ map to RITA. The cosmid, BAC and PAC clones agree with the physical map of chromosome 19 of the Lawrence Livermore National Laboratory. The encircled numbers refer to the newly established STS marker RSTS1-RSTS 10 (for comparison see Table 2);

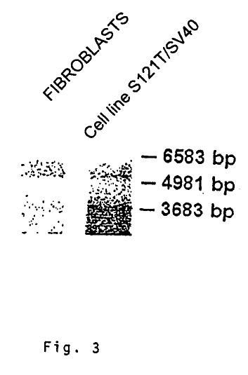

FIG. 3 shows the results of a Northern Blot hybridisation on RNA isolated from thyroid adenoma cell line S121/SV40 and fibroblasts, using a 1092 bp cDNA probe of exon 1 to exon 5. The 5.5 kb and 6.2 kb transcripts were found in the fibroblasts and the adenoma cell line; and

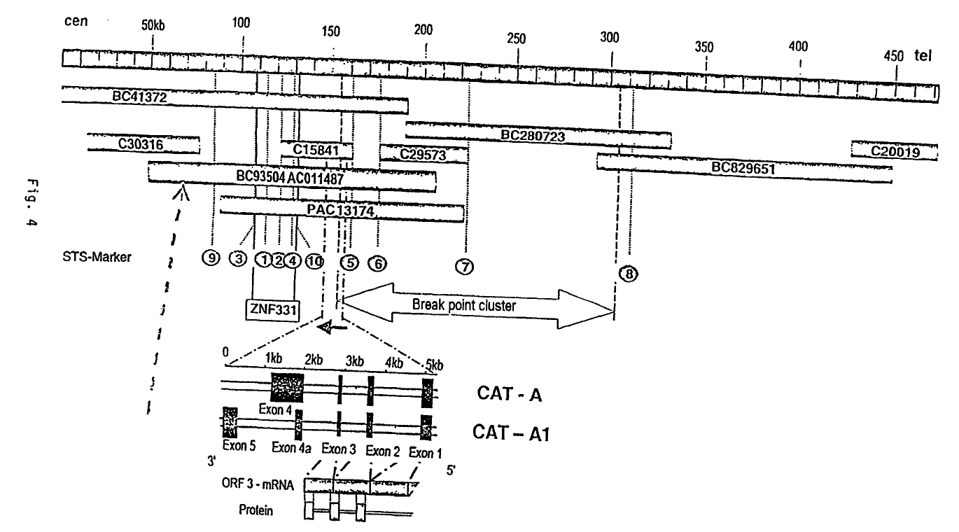

FIG. 4 shows the genome position of the CAT-A gene;

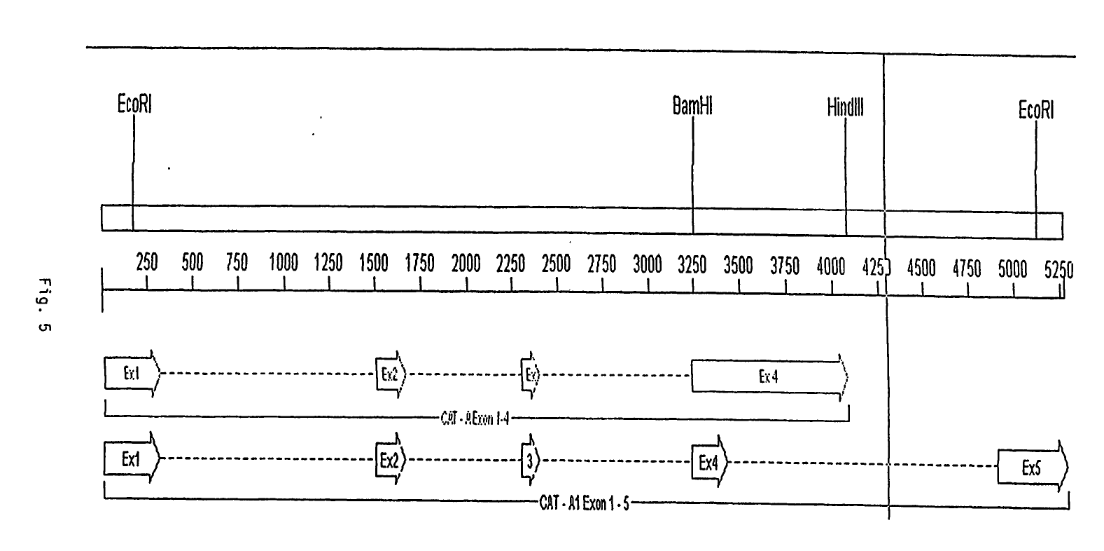

FIG. 5 shows the genome organisation of CAT-A;

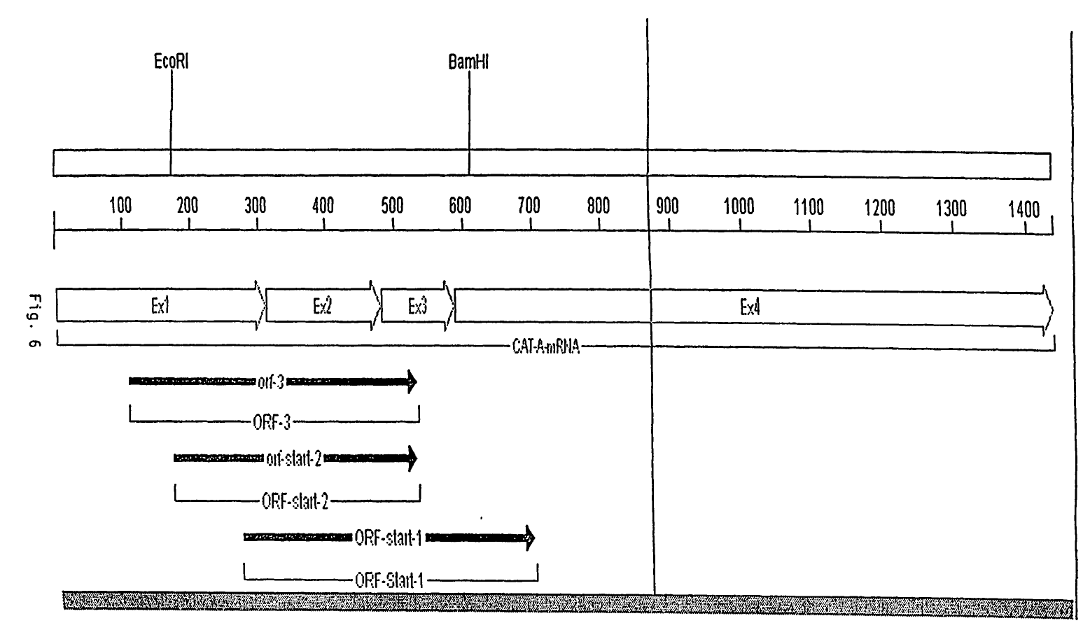

FIG. 6 is an organisational sketch of the mRNA of CAT-A;

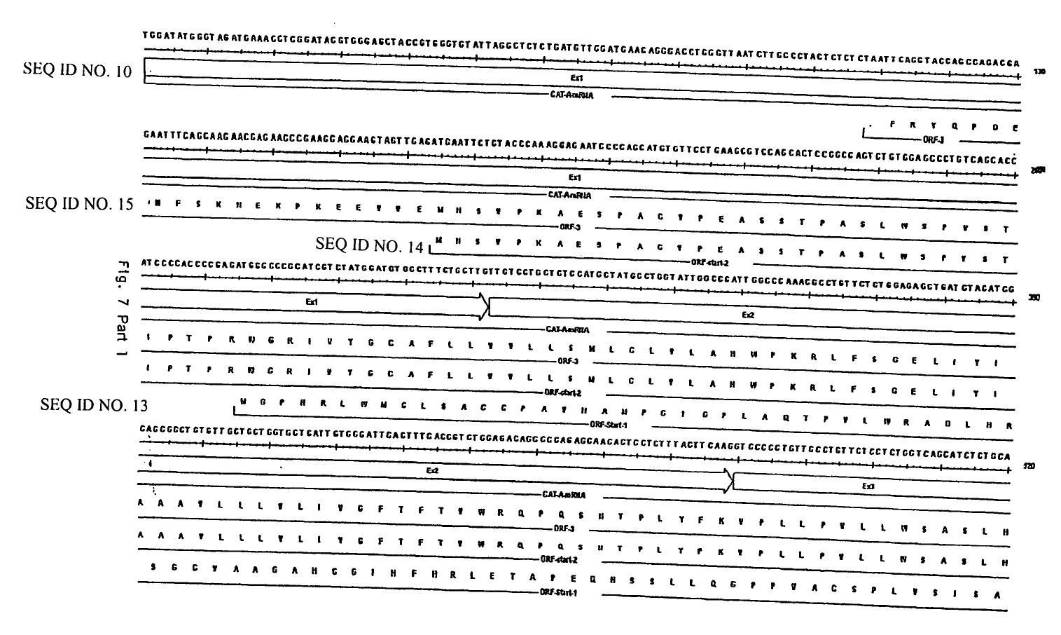

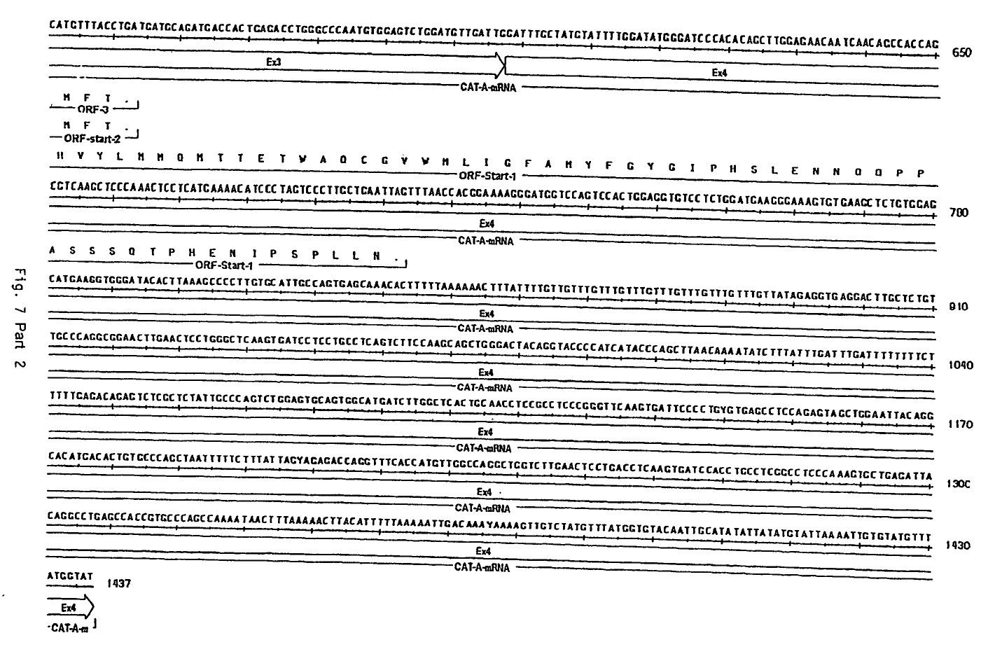

FIG. 7 shows the various open reading frames as well as open reading frames with possible amino acid sequences designated herein as ORFstart1, ORFstart2 and ORF3;

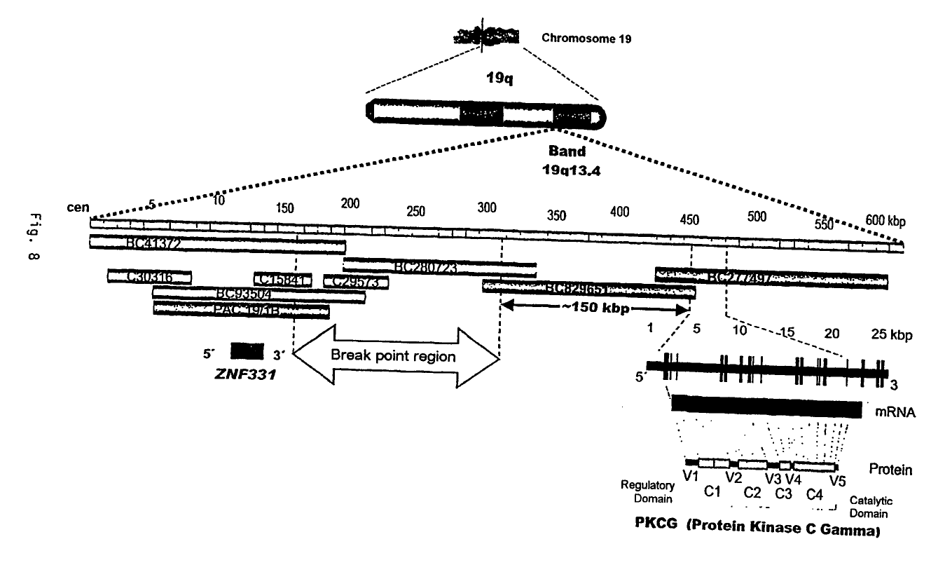

FIG. 8 shows the position of the PKCG gene;

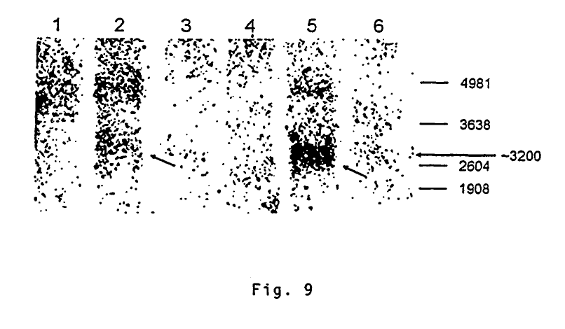

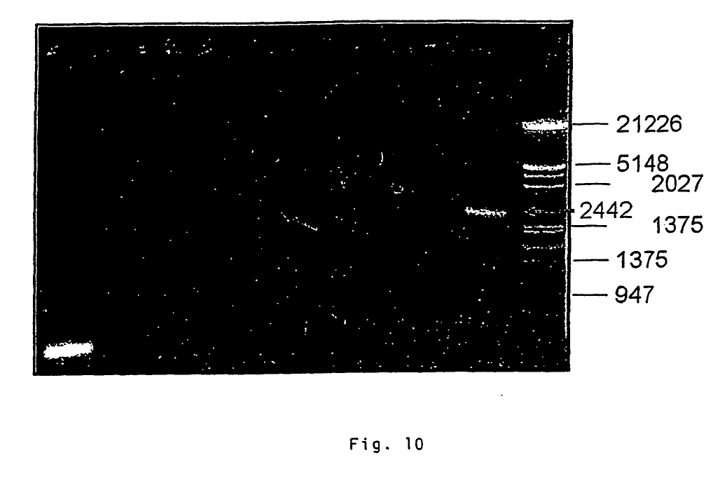

FIG. 9 is a Northern Blot to detect PKCG on thyroid carcinoma cells and thyroid normal tissue;

FIG. 10 is the result of a semiquantitative RT-PCR of the PKCG gene;

FIG. 11 is a diagram of the various genes or gene part sequences described herein for the first time relating to CAT-A or CAT-A1, the DC-2 homologue and protein kinase Cgamma;

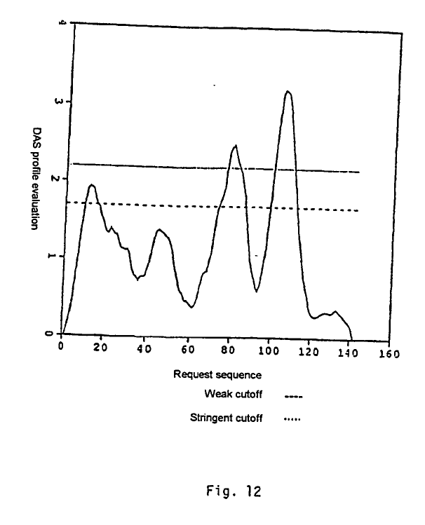

FIG. 12 shows the result of a transmembrane domain analysis using the DAS program of the gene product of ORFstart1;

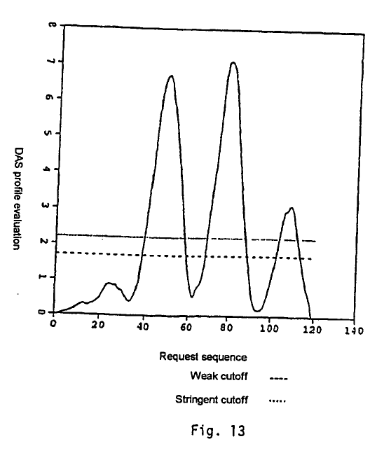

FIG. 13 shows the result of a transmembrane domain analysis using the DAS program of the gene product of ORFstart2;

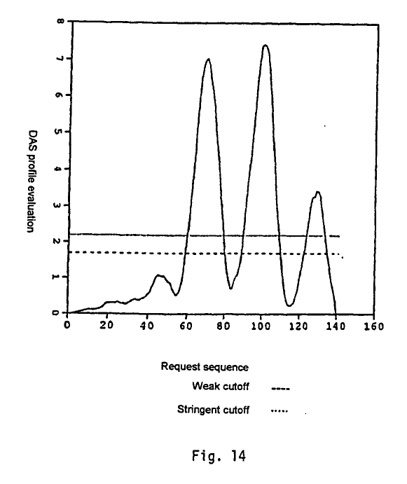

FIG. 14 shows the result of a transmembrane domain analysis using the DAS program of the gene product of ORF3;

FIG. 15 shows the cDNA of CAT-A;

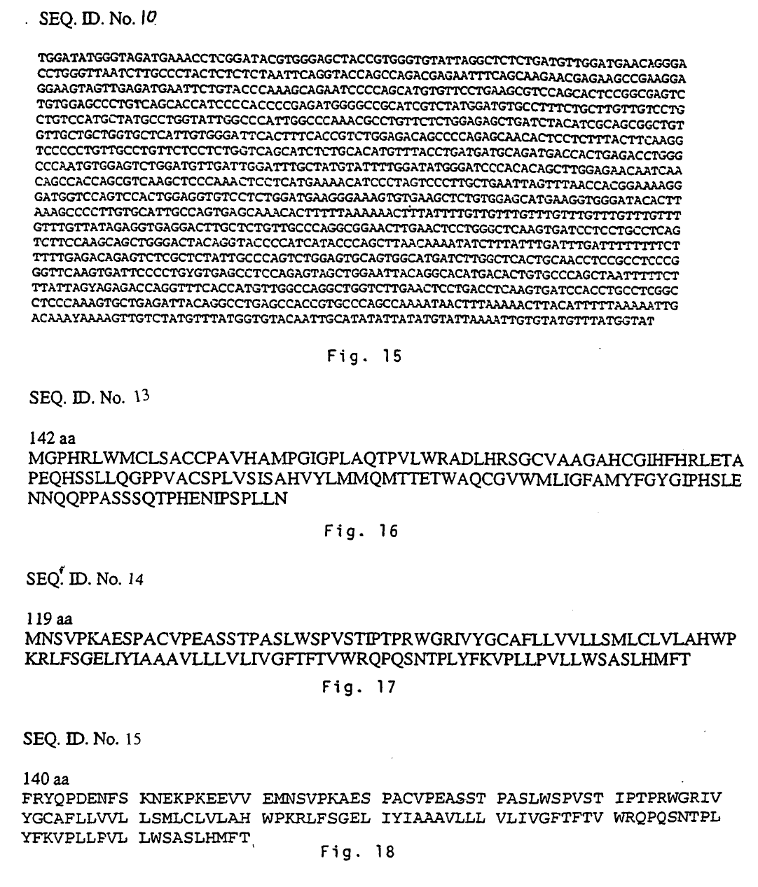

FIG. 16 shows the protein or amino acid sequence of ORFstart1 of CAT-A-cDNA;

FIG. 17 shows the protein or amino acid sequence of ORFstart2 of CAT-A-cDNA;

FIG. 18 shows the protein or amino acid sequence of ORF3 of CAT-A-cDNA;

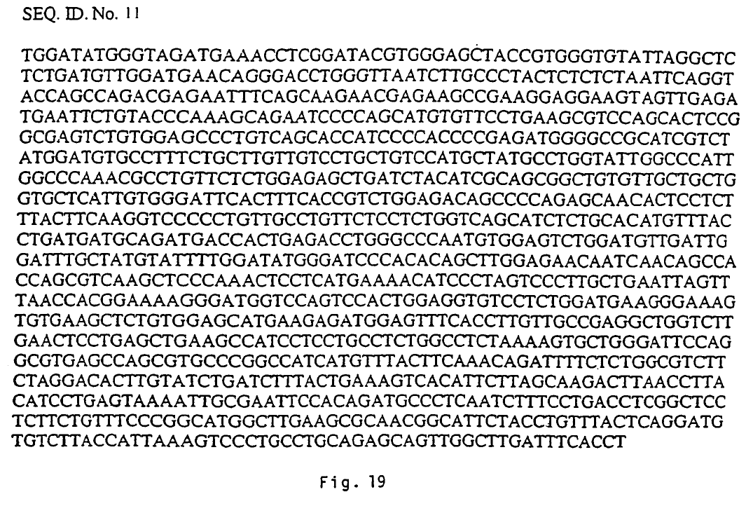

FIG. 19 shows the cDNA of CAT-A1;

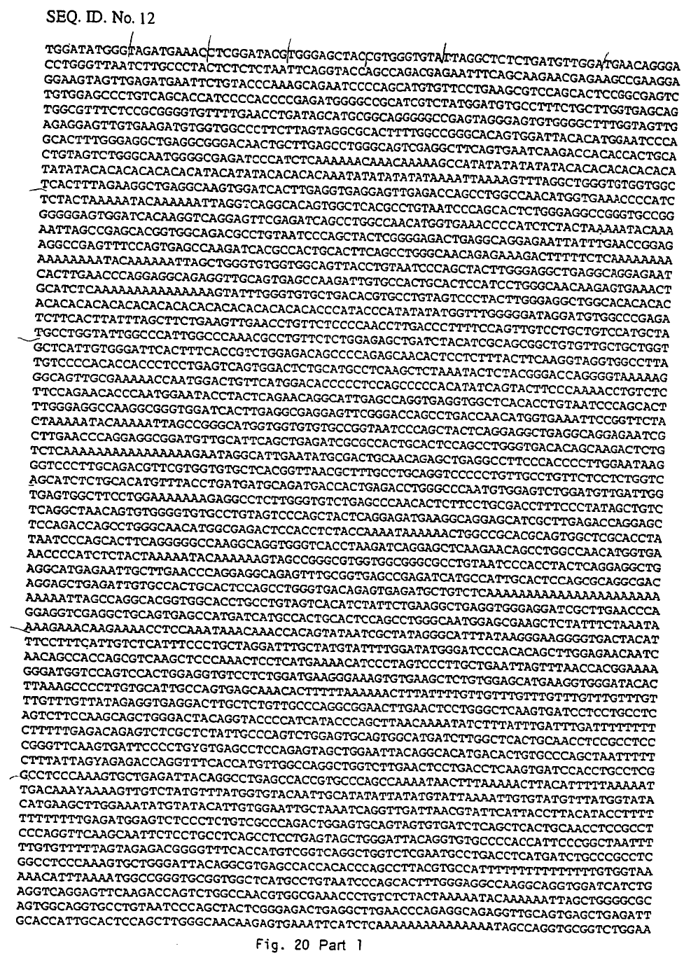

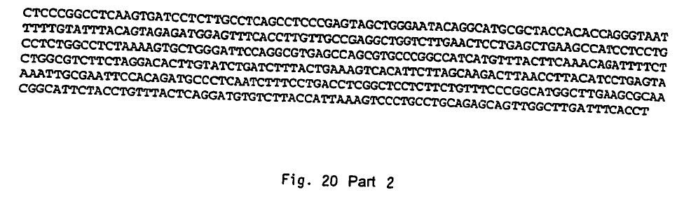

FIG. 20 shows the genome organisation or sequence of CAT-A or CAT-A1 with more extensive sequence information;

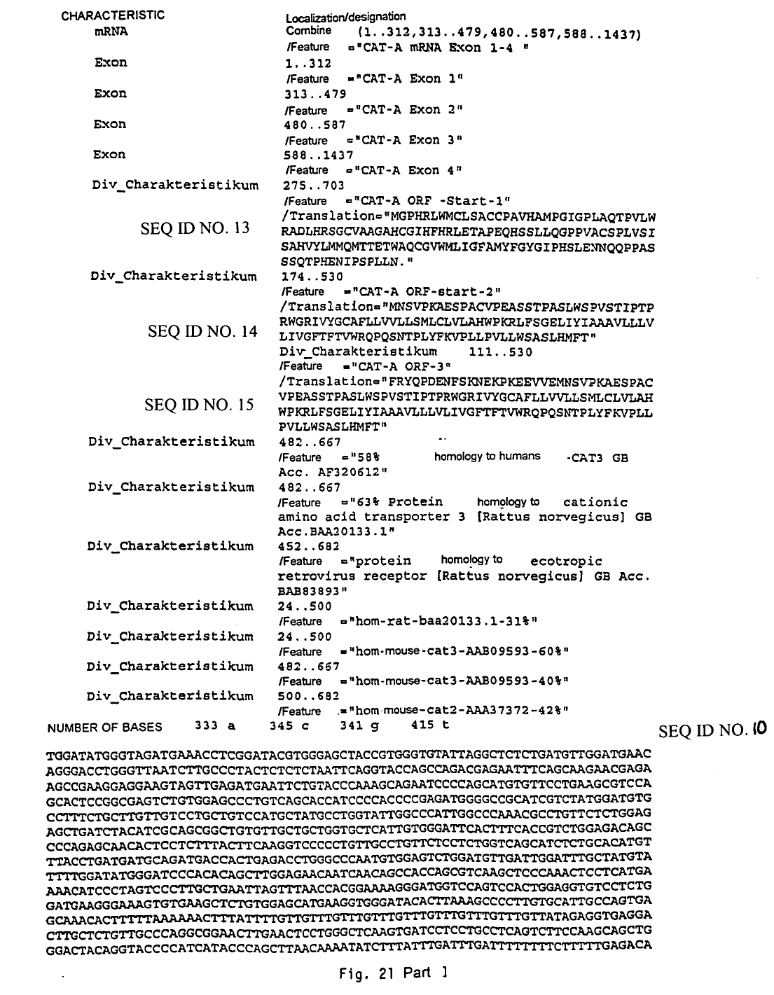

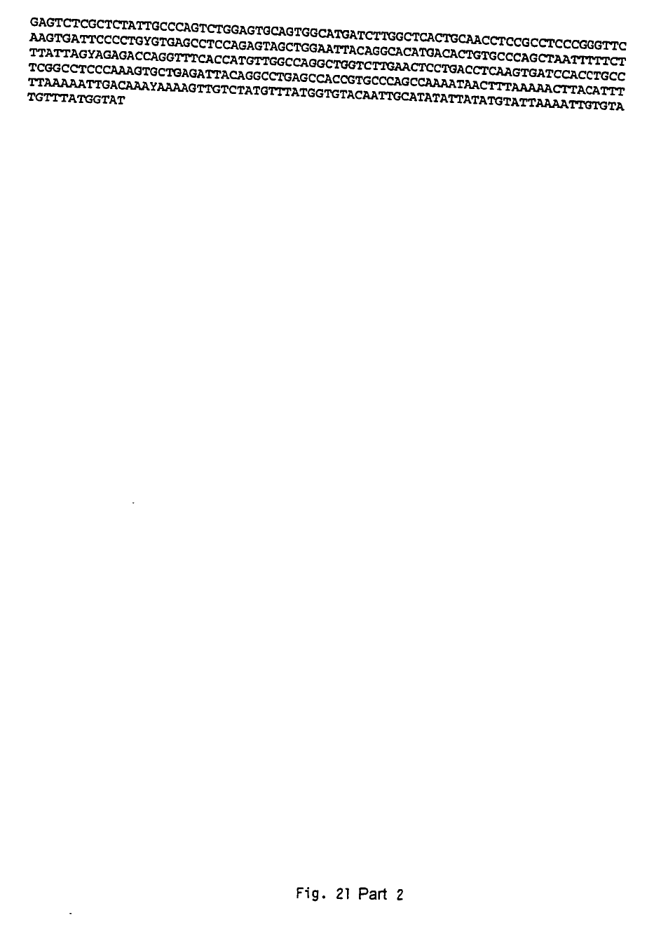

FIG. 21 shows the cDNA of CAT-A with more extensive sequence information;

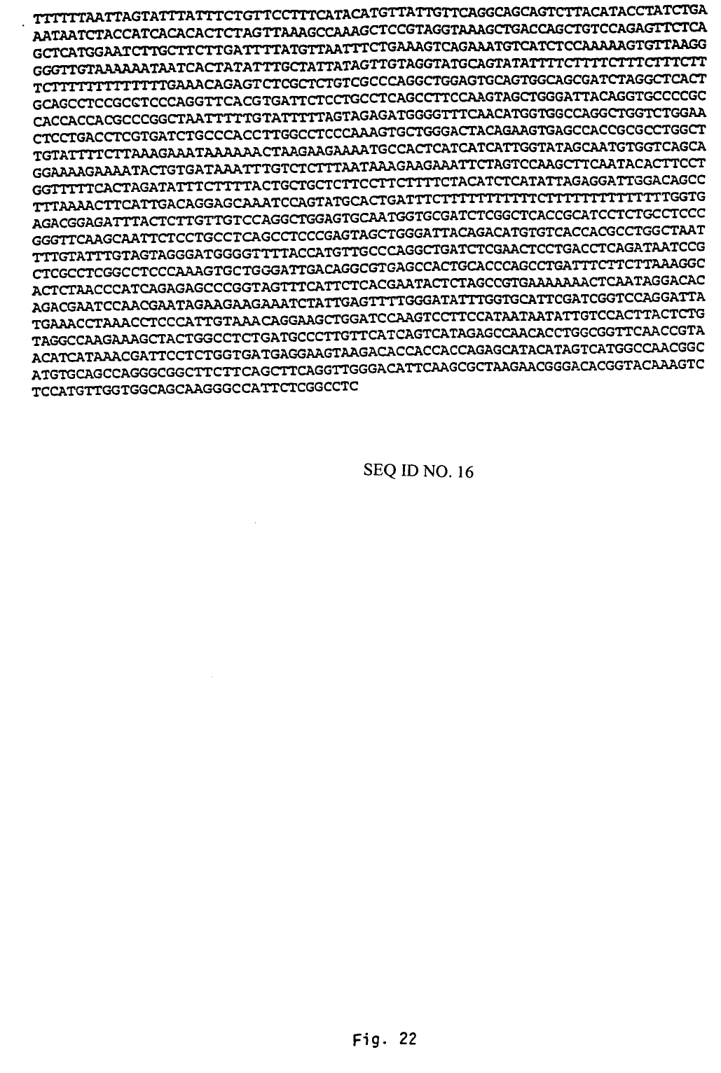

FIG. 22 shows the genome sequence of the DC2 homologous gene according to the invention;

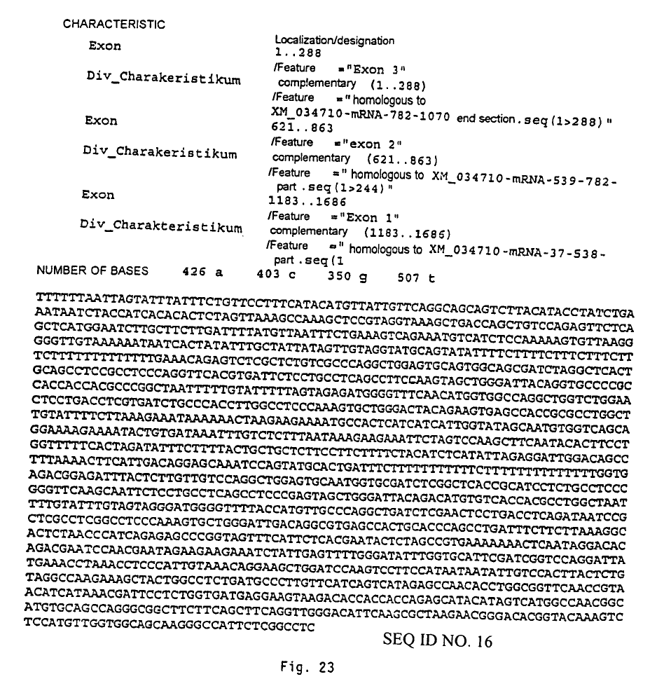

FIG. 23 shows the genome sequence of the DC2 homologous gene according to the invention with more extensive sequence information;

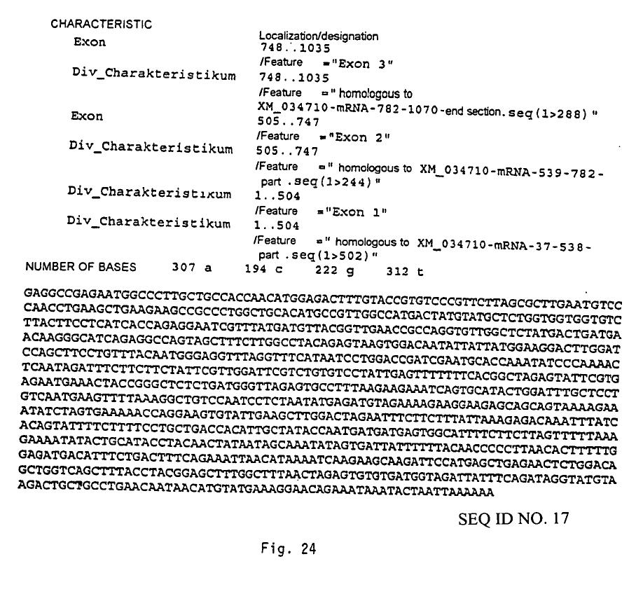

FIG. 24 shows the cDNA off the DC2 homologous gene with more extensive sequence information;

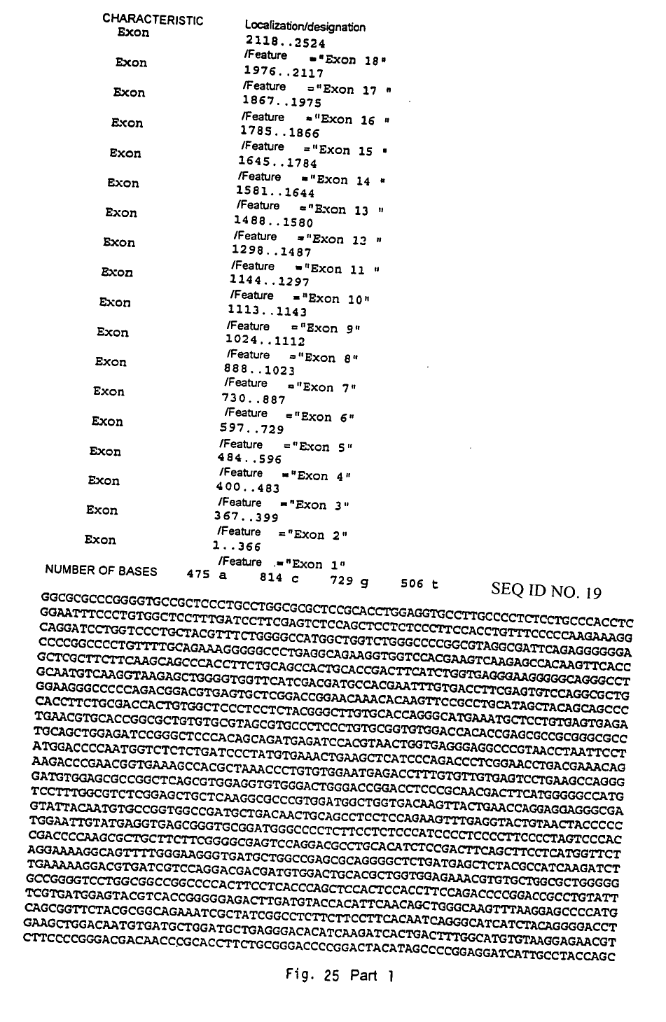

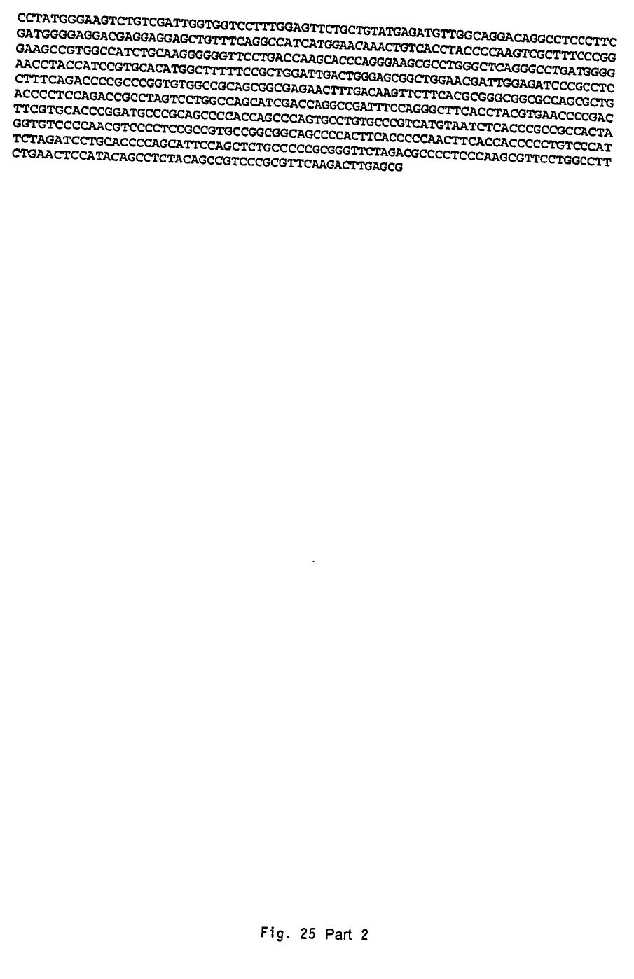

FIG. 25 shows the cDNA of PKCG with more extensive sequence information; and

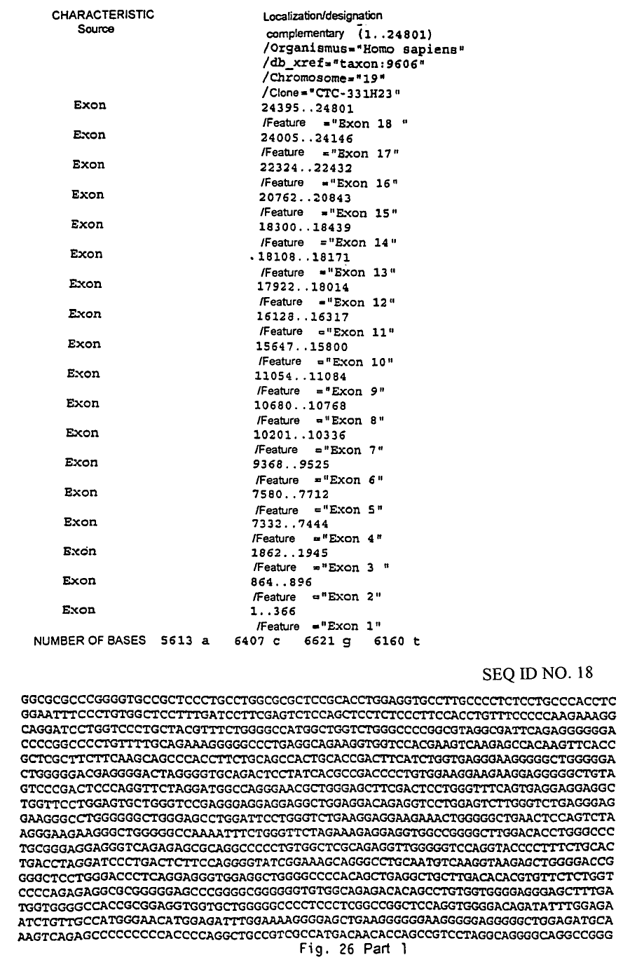

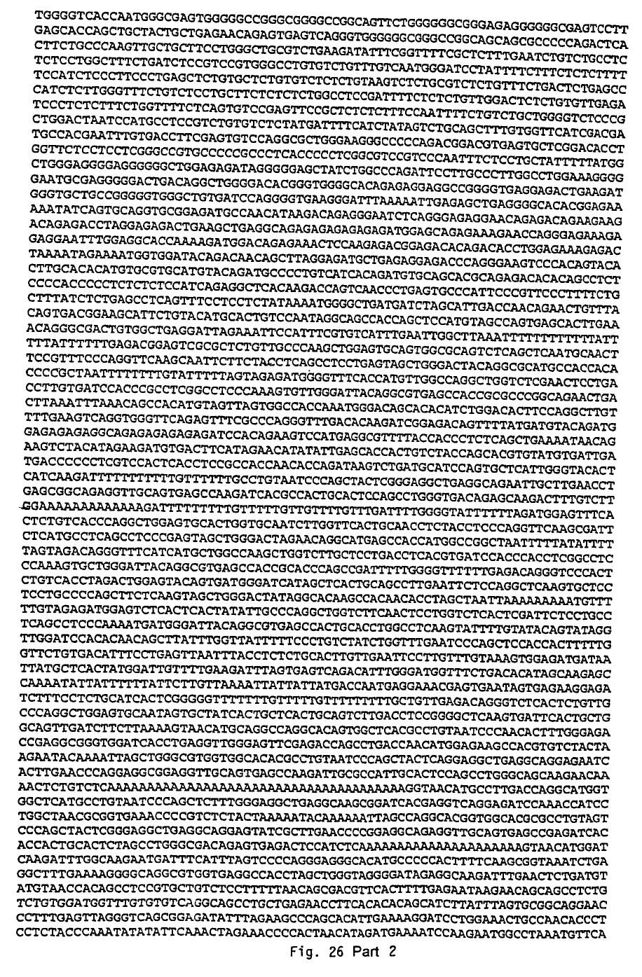

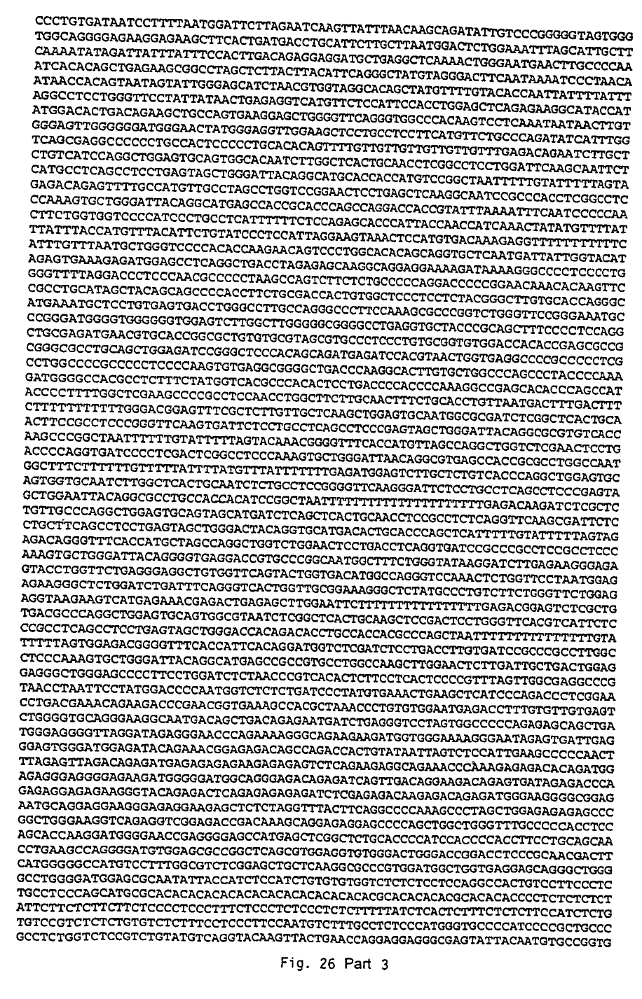

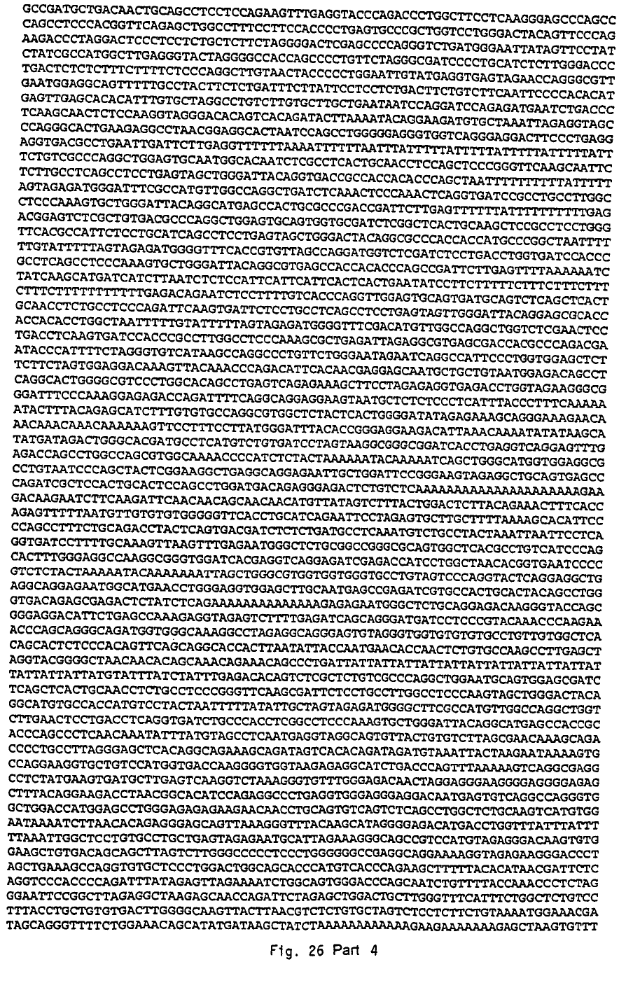

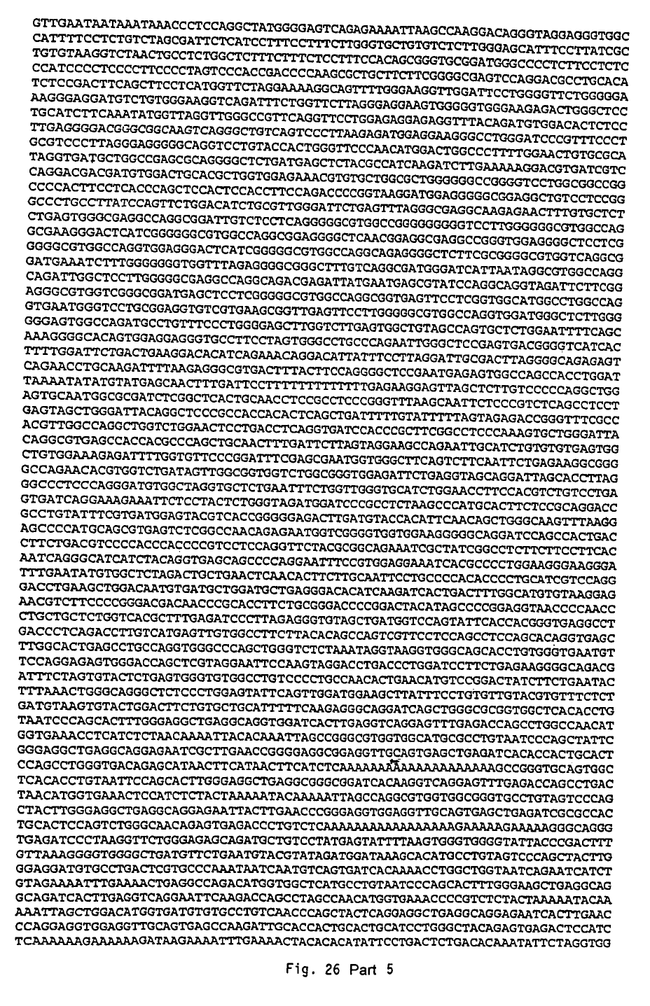

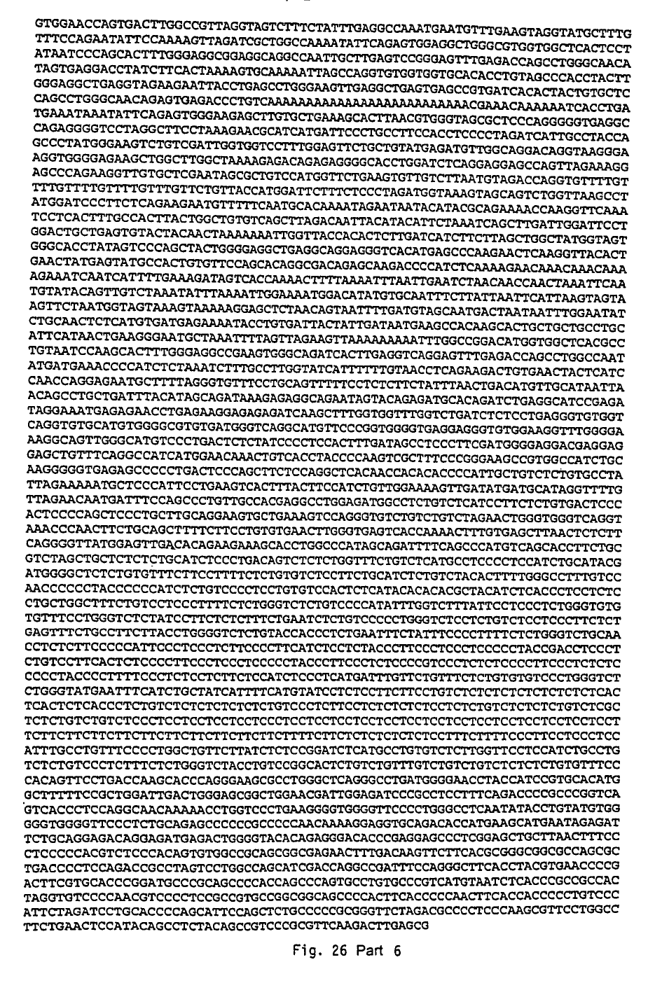

FIG. 26 shows the genome sequence of PKCG with more extensive sequence information.

### EXAMPLE 1

### Cytogenetic Analysis of Thyroid Hyperplasias and Thyroid Adenomas

Thyroid hyperplasias and adenomas are among the frequent changes to the thyroid epithelium. A distinction can be made here between lesions with clonal chromosome changes and those with apparently normal karyotype. There is a very high probability that the cytogenetic changes are associated with tumour genesis. In our investigations we were able to establish clonal aberrations in around 20% of cases. Changes in the chromosomal band 19q13 are the most frequent structural aberrations. The importance of the translocation for the formation and progression of thyroid tumours is so far unknown. By means of molecular genetic investigations we were able to show that the break points at translocations of chromosome 19 in the band 13q cluster in a genome region of 150 kbp.

Cell culture and chromosome analysis were carried out using the methods already described for pleomorphic adenoma of the parotid gland (Bullerdiek et al., 1987). The karyotypes are described in accordance with the ISCN of 1995. The cell lines were obtained by transformation with a construct containing the SV40 “early region” (Belge et al., 1992). Two new cell lines were produced from the primary tumours S172 and S290.1. These cell lines and four primary tumours were used in FISH studies. The karyotype descriptions are given in Table 1. For the localisation of the break point there was . . . the PAC clone 13174 (Genome Systems, USA), the cosmid clones 15841 and 29573 and the BAC clones 41372, 28072 and 82951, which are described using the Lawrence Livermore National Laboratory (LLNL) nomenclature (Ashworth et al., 1995). The cosmid clones were obtained from the human chromosome-19-specific “flow-sorted” Cosmid Library of the LLNL (deJong et al., 1998; Trask et al., 1992; Ashworth et al., 1995). The BAC clones originate from the human library CIT-HSPC and were isolated by the LLNL. Cosmid and BAC fragments were digested with E-coRI, segregated in 0.8% agarose gels, cut out from the gel, isolated from the gel piece using glass bead technology (QIAExII, QIAGEN, Hilden, Germany) and cloned in the pGEM11zf(+) vector (Promega, Mannheim, Germany). Plasmid, cosmid, PAC and BAC-DNA was isolated using a standard method for alkali lysis and purified over anion exchanger columns (QIAGEN, Hilden, Germany). DNA digestion was carried out using a standard procedure taking into account manufacturer's information. Sequencing was carried out on a 373 DNA sequencer (Applied Biosystems, Weiterstadt, Germany) using vector-specific and insert-specific primers. For the positional localisation of the STSs, cosmids, PACs and BACs, their DNA was digested with the restriction enzymes EcoRI, BamHI or HindIII, segregated in 0.8% agarose gels and transferred onto nylon membranes of the type Hybond N+ (Amersham Pharmacia, Freiburg, Germany). PCR-amplified, DIG-dUTP-labelled (Roche; Mannheim, Germany) STS or plasmid DNAs were used as probes. The labelling was carried out by random-primed incorporation of the DIG-dUTPS in the DNA. ExpressHyb hybridising solution (Clontech Laboratories, Palo Alto, USA) was used for the pre-hybridisation and for the hybridisation. The probes were visualised using chemiluminescence (Roche, Mannheim, Germany). The polymerase chain reaction (PCR) was carried out using the temperatures and primers as specified in Table 2. PCR analyses were carried out using standard procedures. Around 60 ng of template DNA was amplified in a volume of 50 μl with 400 ng of each primer, 300 μM dNTPs, 1×PCR buffer (contains 1.5 mM MgCl₂) and 2.5U AmpliTaq (Perkin Elmer Applied Biosystems, Weiterstadt, Germany). The PCR products were segregated on 1.5% agarose gels, cut from the gel, isolated from the gel using glass bead technology (QIAExII, QIAGEN, Hilden, Germany) and cloned in the pCR2.1 vector (Invitrogen, San Diego, Calif.). Fluorescence in-situ hybridisation (FISH) analyses were carried out by a GTG banding of the same metaphases. The treatment of the metaphases with subsequent FISH studies was carried using the protocol put forward by Kievits et al. (1990). Cosmid, PAC and BAC-DNA was labelled with biotin-14-dATP (Gibco BRL, Life Technologies GmbH, Eggenstein, Germany) using the Nick translation method to produce the probes. Twenty metaphases were studied per cosmid, PAC or BAC probe with the exception of the case S532.1 for which only two metaphases could be evaluated. The chromosomes were counterstained with propidium iodide, analysed using a Zeiss Axiophot fluorescence microscope using an FITC filter (Zeiss Oberkochem, Germany) and recorded using the PowerGene Karyotyping System (PSI, Halladale, U.K.). The FISH studies were carried out on four primary tumours of the thyroid (S476.1, S141.2, S172, S532.1) and on two cell lines from thyroid hyperplasias. The cell lines, like the primary tumours, exhibit a change in the chromosomal band 19q13. The results of these analyses showed that in all the primary tumours the break points are localised in a region spanned by the BAC clone 280723. With this BAC clone signals were obtained both on the normal chromosome 19 and on the two derivative chromosomes.

In further FISH studies signals proximal to the break points were obtained with the cosmids 15841 and 29573, PAC 13174 and BAC 41372 whereas BAC 829651 showed signals distal to the break points. The cell line from the primary tumour S172, i.e., cell line S172/SV40, after hybridisation with the BAC clone 280723, also showed signals on the normal chromosome 19 and on both derivative chromosomes. The cell line S290.1/SV40 during hybridisation with the PAC clone 13174 showed signals on the derivative chromosomes and on the normal chromosome 19. This means that the break point in chromosome 19 for all the tumours studied is localised in a segment of around 150 kbp, corresponding to the sequence between the cosmid 15841 and the BAC clone 829651. For further characterisation of the region the BAC clones 93504, 41372, 280723 and 829651 (LLNL), the PAC clone 13174 (Genome Systems, USA) and the cosmid clones 30316, 15841, 29573 and 20019 (LLNL) were brought together in a contig with the aid of restriction mapping and incorporation of sequencing data. Ten new STSs were produced from this sequence data, which were either obtained by sequencing the insert ends for plasmids or cosmids or by internal sequencing for BAC, PAC and cosmid clones. Oligonucleotide sequences for these PCR attachments as well as the product sizes of the PCR amplificates are listed in Table 2. The contig comprises 470 kbp in length, three of the STSs lie within the break point region, one lies below and six STSs are localised proximally to the break point.

Sequence homologies to three ESTs were found in the immediate vicinity of or within the break point region by sequence analysis methods. A comparison of the ESTs with the genome sequence of the region allowed the structure of this gene to be reconstructed. An EST (accession no. BE271492, AF201937) lies within the 150 kbp break point region, i.e., between the cosmid clones 15841 and 29573. The cDNA of BE271492 was isolated from the ovarian adenocarcinoma cell line library NIH_MGC_{—}9. The cDNA of AF201937 was isolated from dendritic cells (see gene bank entry for AF201937). This contains three exons, 469, 250 and 80 bp long, separated by one approximately 320 bp and one approximately 340 bp long intron. This gene is oriented in the telomere direction. The exons exhibit approximately 95-85% homology with sequences of the break point region. The mRNA of this gene codes for a protein, DC2, that is ubiquitarily expressed. The cDNA of the gene could be isolated from a wide range of tissue types such as fat tissue: brain, intestine, eye, heart, liver, kidney, thyroid, testis, skin; etc.

Another EST (BE564764, BF029168) begins approximately 4 kb upstream of the STS marker RSTS5 on the cosmid clone 15841. Both ESTs originate from the library NIH_MGC_{—}53 which was produced from a bladder carcinoma cell line. The orientation of the ESTs is directed towards the centromere and thus this gene lies with its 3′UTR still within the break point region. On comparison with the genome region an exon/intron structure could be shown. This gene extends, as far as can be seen from the sequence data, over at least 5 kbp. The first three exons exhibit 100% homology with the compared genome sequence, exons 4 and 5 exhibit 93% and 68% homology respectively. Over a section of 110 bp these ESTs exhibit 85% homology with the gene for the cationic amino acid transporter (CAT) of rattus norwegicus. The introns vary in size from around 500 bp to around 2 kbp.

A third EST (AF345987, X62533) was found below the break point.

The homology to the genome sequence below the break point region is approximately 100% as far as is comparable. The orientation of the relevant gene is directed towards the centromere. This gene codes for the protein kinase C gamma. The first known exon begins around 145 kbp downstream of the break point region. On this exon lies the start point for the translation around 1100 bp upstream of the exon. An alternative start point lies around 1600 bp upstream of the known sequence. A comparison with the genome sequence allowed an intron/exon structure to be constructed. As far as can be seen from the data, this gene consists of at least six exons. It is predicted that more exons lie 5′ of the known sequence, i.e., in the direction of the break point region. For the first two exons a size estimate of around 500 bp for intron 1 and around 1000 bp for intron 2 can already be given. PKCgamma cDNA has so far been isolated from brain and gastric tissue, and leucocytes.

The cDNA or mRNA sequences of the afore-mentioned ESTs were checked for expression in a Virtual Northern (SAGE, serial analysis of gene expression; http://www.ncbi.nlm.nih.gov/SAGE/).

During this online analysis selected sequences of ESTs (tags) were compared with the sequences ordered in libraries. Although these results rather represent the frequency of the tag and less the actual gene expression, it is however a reliable instrument for first evaluations. Tags of the ESTs BE271492 and AF201937 (DC2) are found in many libraries, and particularly strong “expression” is found in libraries of medulloblastomas, mamma-adenocarcinomas, normal vascular endothelium cell lines, normal skin and prostatic carcinomas. Tags of the ESTs AF345987 and X62533 (PKCgamma) were only found in two libraries produced from normal brain or astrocytoma cell lines. Tags of the ESTs BE564764 and BF029168 could not be found in the libraries.
Ashworth L K, Batzer M A, Bradriff B, Brandscop E, de Jong P, Garcia E, Garnes J A, Gordon L A, Lamardin J E, Lennon G, Mohrenweiser H, Olsen A S, Slezak T, Carrano A V: An integrated metric physical map of human chromosome 19. Nature Genet 11: 442-427 (1995).
de Jong P J, Yokabata K, Chen C, Lohman F, Pederson L, McNinch J, van Dilla M: Human chromosome-specific partial digest libraries in lambda and cosmid vectors. Cytogenet. Cell Genet 51: 985 (1985).
Bullerdiek J, Böschen C, Bartnitzke S: Aberrations of chromosome 8 in mixed salivary gland tumors: cytogenetic findings on seven cases. Cancer Genet Cytogenet 24: 205-212 (1987).
Belge G, Kazmierczak B, Meyer-Bolte K, Bartnitzke S, Bullerdiek J: Expression of SV40 T-antigen in lipoma with a chromosomal translocation t(3; 12) is not sufficient for direct immortalization. Cell Biol Int Rep 16:339-347 (1992).
ISCN (1995): An International System for Cytogenetic Nomenclature. Mitelman F (ed.) (S Karger, Basel 1995).
Kievits T, Dauwerse J G, Wiegant J, Devilee P, Breuning M H, Cornelisse C J, van Ommen G J B, Pearson P L: Rapid subchromosomal localization of cosmids by nonradioactive in situ hybridisation. Cytogenet Cell Genet 53:134-136 (1990).

Trask B, Christensen M, Fertitta A, Bergmann A, Ashworth L, Branscomp E, Carrano A, Van den Engh G: Fluorescence in situ hybridization mapping of human chromosome 19: Mapping and verification of cosmid contigs formed by random restriction enzyme fingerprinting. Genomics 14: 162-167 (1992).

TABLE 1
Arrangement of DNA probes (cosmid, PAC, BAC)
used for the FISH studies on four
benign primary thyroid tumours and two SV40
transformed cell lines derived
from benign thyroid tumours, showing
all aberrations relating to the band 19q13

| | | | Position of |
|---|---|---|---|
| | | | FISH signals |
| | | | relative to |
| Tumour No. | DNA probe | Translocation | break point |
| S141.2 | c15841 | t(2; 19) (p12; q13) | Proximal |
| c29573 | | | Proximal |
| BAC 41372 | | | Proximal |
| BAC 280723 | | | Spanning |
| BAC 829651 | | | Distal |
| PAC 13174 | | | Proximal |
| S172 | c29573 | t(2; 19) (p12; q13) | Proximal |
| BAC 41372 | | | Proximal |
| BAC 280723 | | | Spanning |
| BAC 829651 | | | Distal |
| PAC 13174 | | | Proximal |
| S476.1 | c29573 | t(5; 19) (q13; q13) | Proximal |
| BAC 41372 | | | Proximal |
| BAC 280723 | | | Spanning |
| BAC 829651 | | | Distal |
| PAC 13174 | | | Proximal |
| S172/SV40 | BAC 280723 | t(2; 19) (q13; q13) | Spanning |
| BAC 829651 | | | Distal |
| S290.1/SV40 | c30316 | t(11; 19) (q23; q13) | Proximal |
| c15841 | | | Proximal |
| BAC 41372 | | | Proximal |
| PAC 13174* | | | Spanning |
| BAC 829651 | | | Distal |
| S532.1 | BAC 280723 | t(14; 19) (q22; q13) | Spanning |

TABLE 2

Primers for PCR amplification of STS markers which
were physically mapped

| | | |
|---|---|---|
| Marker | | |
| gene bank | | Prod- |
| Accession | Forwards | uct |
| number Primer | Primer reversed | (bp) |

| | | |
|---|---|---|
| RSTS1G64894 | TCCTGGCTCATAATTCCATAACCCTTGGT | 423 |
| | GTGCGCCACCTTTTCAGAGTTCTTTTGT | |
| RSTS2G64900 | TTATGCCTGATTCAGTGACACTACTTTTTC | 397 |
| | CGGCTGCCTCTAACATACGGAAGATTC | |
| RSTS3G64895 | GAGGCTGACGCGGCGGCTCTATCTC | 113 |
| | AGCGGTTGCGTCACCGGTGCAGAAG | |
| RSTS4AF260531 | ATATACGTGTATCAGTTTCAGAATGC | 188 |
| | TATTTTAAAGTCAGACATGAAAAAGG | |
| RSTS5AF260970 | GGGCCAGGAAAATGCACGGAAGTGAAG | 360 |
| | CCCGGGCTTGTGGAATTAACTGAGCAG | |
| RSTS6G64896 | TTATGTGGGCCCCAAACCATTACTGATACTC | 385 |
| | CTCATGCCCATAAACCCCTAAATTCCTTGT | |
| RSTS7G64897 | GCCTGCTAAGCCTCTGTGCCTAGTAAAG | 189 |
| | GGTATCAGAAAGGTACAATCCCTATGTCTC | |
| RSTS8G64901 | CCCAAAAGATCCCAAGTCCAGGCAGAAAG | 397 |
| | CCTGGGAAGCTCACAAGGGTGGAAGAC | |
| RSTS9G64898 | GTACATTGGCATCCGCAGGGGTAAC | 406 |
| | CCTCTCAAGGCGTGCTTTTTCGTAAGA | |
| RSTS10G64899 | AGCCCGCTCGTATCTATGG | 301 |
| | TGAGGACTACTTGGGCACAGG | |

### EXAMPLE 2

### Characterisation of a Human CAT-Homologous Gene

During the characterisation of the genes occurring in the break point region of thyroid tumours a new human gene was found which, on the basis of the corresponding sequence of rattus norwegicus, is homologous to the CAT genes. CAT genes are those genes which code for transporter proteins of cationic amino acids. The gene discovered by the present inventor is herein designated in the following as CAT-A or CAT-A1. CAT-A1 is a splice variant of CAT-A as is further verified below.

The CAT-A or CAT-A1 gene lies precisely in the region q13.4 of human chromosome 19. It lies partly in the break point region of the thyroid tumour. The CAT-A/A1 gene lies in the BAC CTB-167G5 (BC93504) with the gene bank number AC011487. The gene direction points to the centromere i.e., the 3′-end is organised towards the centromere.

An overview of the position of CAT-A is given in FIG. 4.

The mRNA of CAT-A is currently given with 1437 nucleotides and is represented as cDNA in SEQ. ID. No. 10. It should be noted that CAT-A and CAT-A1 are currently substantially shorter than the members of the human CAT family.

### EXAMPLE 3

### Genomic Organisation of CAT-A

The genomic organisation of CAT-A and its splice variant CAT-A1 is shown in FIG. 4. The genomic DNA sequence of the homologous gene to the human DC2 already known in the prior art, which is disclosed herein for the first time, is given as having a length of 5278 base pairs. It should be noted that at the present time the 5′-end has not yet been determined. However, starting from the disclosure made herein, it is possible to carry out further sequencing and in this respect complement the sequence. On the other hand, it is recognised by those skilled in the art that on the basis of the information given herein, completion is possible and especially the various representatives of the compound classes of the peptides, proteins, antibodies, anticalins and functional nucleic acids can already be produced or selected on the basis of the disclosure given herein.

In the case of CAT-A, a total of four exons are known, with exon 1 being 312 base pairs long, exon 2 being 167 base pairs long, exon 3 being 108 base pairs long and exon 4 being 850 base pairs long.

CAT-A1 on the other hand has five exons, which arise because exon 4 comprises an alternative splice site. In this respect, exons 1 to 3 of CAT-A1 are identical to the first three exons of CAT-A. The fourth exon of CAT-A1, i.e., exon 4a, however, is only 200 base pairs long and exon 5 of CAT-A1 is 381 base pairs.

Characterisation of the mRNA for CAT-A

The length of the CAT-A mRNA is currently given as 1437 nucleotides. In this case, two reading frames exist for which one start codon each exists. The two reading frames and the translation products thereof are designated herein as ORFstart1 and ORFstart2. A third reading frame, likewise contained in CAT-A, herein designated as ORF3 has no start codon, i.e., the sequencing has not yet progressed to the extent that this could have been uniquely identified and contains ORFstart2. ORF3 and ORF2 thus have the same reading frames.

Characterisation of the CAT-A1 mRNA

The CAT-A1-mRNA currently has a length of 1168 nucleotides. This differs from the CAT-A mRNA in having a different 3′-end which however possibly does not code. The sequence hereto is disclosed herein as SEQ. ID. No. 11.

### EXAMPLE 4

### Characterisation of the Different Reading Frames of the CAT'A mRNA

1. Characterisation of the Reading Frame ORFstart1

Possible transmembrane domains of the different reading frames were determined using a total of three different software programs. The corresponding software programs are “DAS” Transmembrane Prediction which can be downloaded via the following URL address: http://www.sbc.su.se/˜miklos/DAS/. This method was developed by Cserzo et al. (M. Cserzo, E. Wallin, I.

Simon, G. von Heijne and A Elofsson: Prediction of transmembrane alpha-helices in procariotic membrane proteins: the Dense Alignment Surface method: Prot. Eng. vol. 10, no. 6, 673-676, 1997).

According to the result shown in FIG. 12, the ORFstart1 reading frame has two transmembrane domains.

Another method for determining transmembrane domains is provided by the so-called HMMTOP server. HMMTOP is an automatic server for the prediction of transmembrane helices and topologies of proteins as developed by G. E. Tusnady from the Institute of Enzymology. The corresponding URL address is: http://www.enzim.hu/hmmtop/index.html. The method is also described in Tusnády, G. E. et al. (G. E. Tusnady and I. Simon (1998) Principles Governing Amino Acid Composition of Integral Membrane Proteins: Applications to Topology Prediction. J. Mol. Biol. 283, 489-506).

The result of the HMMTOP servers relating to the reading frame ORFstart1 can be summarised as follows. Two transmembrane helices are also identified here and allocated to the positions 72-88 or 95-112. The N-terminus is arranged outwards, i.e., extracellularly.

Another method for predicting transmembrane regions is the so-called TMpred program. The algorithm is based on a statistical analysis of TMbase, a database of naturally occurring transmembrane proteins. The prediction is made using a combination of a plurality of weighted matrices. The corresponding URL address is: www.ch.embnet.org/software/TMPRED form.html. The method is also described by Hofmann et al. (K. Hofmann & W. Stoffel (1993) TMbase—A database of membrane spanning protein segments Biol. Chem. Hoppe-Seyler 374, 166).

As a result of using TMpred, two models are considered, differing in respect of the number of transmembrane helices. In one clearly preferred model, the N-terminus is intracellularly oriented and comprises three strong transmembrane helices. The positions 1-21 exhibit an inside to outside orientation, the amino acids of positions 66-87 exhibit an outside to inside orientation and amino acids 95-114 exhibit an inside to outside orientation.

The model proposed as an alternative has two strong transmembrane helices, the first helix comprising the amino acid positions 8-30 and having outside to inside orientation and the second helix comprising the amino acid positions 95-114 with inside to outside orientation.

2. Characterisation of the Reading Frame ORFstart2

The amino acid sequence encoded by this reading frame is given herein as SEQ. ID. No. 14.

Characterisation of the reading frame ORFstart2 using the three software programs for determining transmembrane helices described previously yielded the following result:

Three transmembrane helices were determined using the “DAS” transmembrane protein program. The N-terminus is in this case intracellularly arranged. The length of the amino acid sequence of this reading frame is 119 amino acids.

The result of the applied analysis using the DAS program is shown in FIG. 13.

A more extensive analysis of the reading frame ORFstart2 using the HMMTOP server likewise yielded the presence of three transmembrane helices, namely in the region of amino acids 38-57, 70-89 and 98-117.

Two possible models were again considered using the TMpred method. The strongly preferred model yielded three strong transmembrane helices, the helix of the amino acid positions 38-57 extending with an inside to outside orientation, the helix of amino acid positions 69-90 extending with an outside to inside orientation, and the helix of amino acid positions 99-119 extending with an inside to outside orientation.

The alternative model likewise comprises three strong transmembrane helices, wherein for the formation of the helix the amino acids of positions 38-58 are assumed to have a transmembrane helix with an outside to inside orientation, amino acid positions 72-90 are assumed to have an inside to outside orientation and amino acid positions 100-118 are assumed to have an outside to inside orientation.

3. Characterisation of the Reading Frame ORF3

The protein encoded by this reading frame is herein disclosed as SEQ. ID. No. 15 and comprises a total of 140 amino acids. This reading frame has 34% homology with respect to amino acids 1-130 to the solute carrier family 7 comprising three members (cationic amino acid transporter, Y⁺) system. The corresponding gene bank entry is found under the accession number Gene Bank Acc. AAL37184.

A further homology exists with respect to the amino acids 107-130 to the ectropic retrovirus receptor of rattus norwegicus. A 62% homology is established here. This sequence is found as gene bank entry BAB83893.

A characterisation of this reading frame using the DAS method yielded the existence of a total of three transmembrane domains. The result of the analysis is shown herein as FIG. 14. The analysis using the HMMTOP program also yielded three transmembrane helices formed by amino acids 59-78, 91-110 and 119-138.

An analysis using TMpred finally again yielded three strong transmembrane helices on the basis of two models. In the preferred model these are formed by the amino acids at positions 79-80 with inside to outside orientation, 90-110 with outside to inside orientation and 120-140 with inside to outside orientation.

The alternative model likewise has three strong transmembrane helices, these being formed by the amino acids at positions 59-79 with outside to inside orientation, 93-111 with inside to outside orientation and 121-139 with outside to inside orientation.

In connection with the present invention, especially when using the various translation products of the sequences according to the invention or the polypeptides according to the invention, such as are, among others, the subject of the sequences SEQ. ID. No. 13-15, . . . can function as target molecules. In this case, it is particularly noticeable that transmembrane proteins are involved which is important insofar that during the development of molecules of the various classes of compounds discussed herein, i.e. peptides, proteins, antibodies, anticalins and functional nucleic acids, but also small molecules, the accessibility of parts of these sequences is especially easy insofar as parts of these target molecules are arranged extracellularly, as follows from the previous descriptions of the transmembrane domains.

### EXAMPLE 5

### Overexpression of PKCG in Thyroid Carcinoma Cells

A semiquantitative PCR and a Northern Blot Analysis were used to study the extent to which PKCG was overexpressed in thyroid carcinoma cells.

The semiquantitative RT-PCR was carried out on cDNA of the anaplastic thyroid carcinoma S227, the thyroid adenoma cell line S40.2, normal thyroid tissue, the cell line HeLa, the mammary carcinoma cell line MCF-7 and the cell line EFM-19.

HPKCGup1 (5′-ccttgcccctctcctgcccacctc-3′) and HPKCGlo2 (5′-gggacggctgtagaggctgtatggagttcagaag-3′) were used as primers which include the 2091 bp open reading frame of PKC? on the mRNA level and amplify a fragment of 2424 bp.

In order to compare the RT-PCR of the PKC? gene, an RT-PCR was carried out in parallel on cDNA of the above-mentioned tissue using the GAPDH primers GAPDH2 (5′gtgaaggtcggagtcaacg-3′) and GAPDH3 (5′-ggtgaagacgcccagtggactc-3′) which amplify a product of 299 bp. The attachments (50 μl total volume per attachment) contained 2 μl cDNA, 5 μl 10×PCR buffer, respectively 1 μl primer, 1 μl 2 mM dNTP mix, 1.5 μl 15 mM MgCl₂ and 0.5 μl Taq-polymerase as well as 38 μl twice distilled H₂O.

The RT-PCR conditions were the same for said primer pairs (HPCKGup1/1o2 and GAPDH2/3) except for the annealing temperatures (HPCKGup1/1o2: 68° C.; GAPDH2/3: 52° C. for 30 sec in each case) and were 95° C. (30 sec) for the denaturing and 72° C. (45 sec) for the extension. In total these steps took place in 35 cycles. Before commencement there was a single denaturing at 95° C. (3 min.) and to conclude an additional extension at 72° C. for 10 min.

The RT-PCR was detected by gel electrophoresis.

The Northern Blot analysis was carried out as follows:

A 2.4 kbp PKCG specific fragment which includes the ORF was used as the molecular probe. Radioactive labelling with ³²p was carried out using the principle of “random primer extension”. After pre-hybridising the membrane in ExpressHyb™ hybridising solution (Clontech Laboratories, Palo Alto, U.S.A.) for 30 min at 68° C., the membrane was hybridised with 100 ng of probe in ExpressHyb™ hybridising solution for 60 min at 68° C. The membrane was then washed twice for 20 min at room temperature in 2×SSC/0.05% SDS and four times for 10 min at 50° C. in 0.1×SSC/0.1% SDS. The signals were detected on a STORM Phosphorimager (Molecular Dynamics, Sunnyvale, U.S.A.).

The result of the Northern Blot analysis is shown in FIG. 9 and the result of the semiquantitative PCR in FIG. 10.

The human PKCG gene lies approximately 150 kb from the break point region of the thyroid tumours. Regulatory elements of the PKCG gene can be affected by rearrangement in the break point region. For these regions PKCG is a target molecule in connection with functional disorders of the thyroid and/or hyperplasias of the thyroid and/or thyroid tumours. The relative position of the PKCG gene is shown in FIG. 8. As can be seen from the Northern Blot analysis, PKCG is overexpressed in thyroid carcinoma cells. Detection was carried out using a PKCG-specific probe which was used for hybridisation purposes. The hybridisation conditions herein correspond generally and particularly to those as disclosed in the examples.

The thyroid carcinoma was transferred into a cell line, which also shows a change at chromosome 19 and was the subject of the investigations described in this example.

The results described previously in connection with the Northern Blot analysis were also confirmed using a semiquantitative PCR. The expression in a thyroid carcinoma cell was determined by semiquantitative RT-PCR using PKCG-specific primers. On the agarose gel shown in FIG. 10 the following are plotted: in lane 1—a marker (Marker III (Roche), λ-DNA cut with HindIII-EcoRI), lane 2—a cell line of the anaplastic thyroid carcinoma S277, lane 3—the cell line of the thyroid adenoma S40.2, lane 4—normal thyroid tissue, lane 5—a Hela cell, i.e., mammary carcinoma, lane 6—the cell line MCF7 (ATCC number HTB-22, likewise an adenocarcinoma), lane 7—the cell line EFM19, a human breast cancer cell line (DSMZ number ACC231), lane 8—a negative control, lane 9—a positive control (detection of GAP dehydrogenase to confirm isolation of cDNA).

It thus follows from FIG. 10 that in the case of the anaplastic thyroid carcinoma S277, the expression of the PKCG gene, in the present case having a length of 2442 kb, could be detected. PKCH gene activity could also be detected in the cell line MCF7. The corresponding bands in FIG. 10 are shown by an arrow.

The features of the invention disclosed in the previous description, the sequence protocol and the claims, both individually or in any combination, can be important for the implementation of the invention in its various embodiments. The disclosure content of the claims is hereby taken up by reference.

1. A nucleic acid that has a modified expression in hyperplasias and/or tumours, characterised in that it comprises a nucleic acid sequence which is selected from the group comprising SEQ. ID. No. 1, 2, 7, 10, 11 and 12.

2. The nucleic acid according to claim 1, characterised in that the tumour is selected from the group comprising epithelial tumours with a change in the chromosomal band 19q and tumours with a change in the chromosomal band 19q13.

3. The nucleic acid according to claim 1 or claim 2, characterised in that the hyperplasia is selected from the group comprising hyperplasias of the thyroid.

4. The nucleic acid sequence according to any one of claims 1 to 3, characterised in that SEQ. ID. No. 1, 2, 7, 10, 11 and/or 12 codes for a CAT, especially a human CAT, or a part thereof.

5. A nucleic acid comprising a nucleic acid sequence which, without the degeneration of the genetic code, would code for the same amino acid sequence as a nucleic acid according to any one of claims 1 to 4.

6. A nucleic acid which hybridises to one of the nucleic acids according to any one of claims 1 to 5.

7. A vector characterised in that it comprises a nucleic acid according to any one of the preceding claims.

8-10. (canceled)

11. A polypeptide encoded by a nucleic acid according to any one of claims 1 to 6.

12. (canceled)

13. A cell, preferably an isolated cell which comprises a vector according to claim 7.

14. An antibody characterised in that it is directed against a polypeptide according to claim 11.

15. An antibody characterised in that it is directed against a polypeptide encoded by the nucleic acid of claim 1.

16. A ribozyme characterised in that it is directed against a nucleic acid according to any one of claims 1 to 6.

17. is canceled.

18. An antisense nucleic acid comprising a sequence complementary to one of the nucleic acid sequences according to any one of claims 1 to 6.

19. A RNAi comprising a sequence complementary to or identical to one of the nucleic acids according to any one of claims 1 to 6, wherein preferably the RNAi comprises a region having a length of 21 to 23 nucleotides which is complementary or identical.

20. A method for determining a compound which influences the effect of a translation product of a nucleic acid according to any one of the preceding claims, especially inhibits said effect, characterised by the following steps:
a) Providing the translation product and the compound
b) Bringing into contact the translation product and the compound in a system which represents the effect of the translation product, and
c) Determining whether any modification of the effect of the translation product occurs under the influence of the compound.

21. The method for determining a compound which influences the effect of a transcription product of a nucleic acid according to any one of the preceding claims, especially inhibits said effect, characterised by the following steps:
a) Providing the transcription product and the compound
b) Bringing into contact the transcription product and the compound in a system which represents the effect of the transcription product, and
c) Determining whether any modification of the effect of the transcription product occurs under the influence of the compound.

22. is canceled.

23. The method for determining genes responsible for the formation of hyperplasias and tumours, especially of the thyroid, comprising the following steps:
a) Determination of break points at chromosomal translocations of the hyperplasias and the tumours,
b) Determination of genes which lie within a region of 400 kbp, preferably 150 kbp, in each direction from the break point region, and
c) Determining whether the translation/transcription of the gene in a cell of the hyperplasia or of the tumour is modified compared with a non-hyperplasia cell or a non-tumour cell.

24. Use of a nucleic acid according to any one of claims 1 to 6 and/or a ribozyme according to claim 16 and/or an antisense nucleic acid according to claim 18 and/or an RNAi according to claim 19 for the diagnosis and/or treatment of functional disorders of the thyroid and/or hyperplasias of the thyroid and/or thyroid tumours.

25. Use of a nucleic acid according to any one of claims 1 to 6 and/or a ribozyme according to claim 16 and/or an antisense nucleic acid according to claim 18 and/or an RNAi according to claim 19 for the manufacture of a medicament, especially for the treatment and/or prevention of functional disorders of the thyroid and/or hyperplasias of the thyroid and/or thyroid tumours.

26. Use of a polypeptide according to claim 11 for the diagnosis and/or treatment of functional disorders of the thyroid and/or hyperplasias of the thyroid and/or thyroid tumours.

27. Use of a polypeptide according to claim 11 for the manufacture of a medicament, especially for the treatment and/or prevention of functional disorders of the thyroid and/or hyperplasias of the thyroid and/or thyroid tumours.

28. Use of an antibody according to any one of claims 14 to 15 for the diagnosis and/or treatment of functional disorders of the thyroid and/or hyperplasias of the thyroid and/or thyroid tumours.

29. Use of an antibody according to any one of claims 14 to 15 for the manufacture of a medicament.

30. A kit for the diagnosis of functional disorders of the thyroid and/or hyperplasias of the thyroid and/or thyroid tumours, characterised in that the kit comprises at least one element selected from the group comprising a nucleic acid, a vector, a polypeptide, a cell, an antibody, an antisense nucleic acid, RNAi and a ribozyme, each according to one of the preceding claims.

31. A method for detecting functional disorders of the thyroid and/or hyperplasias of the thyroid and/or thyroid tumours, characterised by the following steps:
a) contacting thyroid material with the agent selected from the group comprising a nucleic acid, a vector, a polypeptide, an antibody, an antisense nucleic acid, RNAi, a ribozyme and a cell, each according to one of the preceding claims, and
b) determining whether functional disorders of the thyroid and/or hyperplasias of the thyroid and/or thyroid tumours are present.

32. (canceled)

33. Use of a nucleic acid according to any one of claims 1 to 6 as a primer and/or a probe.

34. A primer for representing and/or screening and/or detecting a nucleic acid, characterised in that it is complementary to a part of a nucleic acid sequence according to any one of claims 1 to 6.

35. Use of a primer for representing and/or screening and/or detecting a nucleic acid, preferably a nucleic acid according to any one of the preceding claims, characterised in that the primer is complementary to a part of a nucleic acid sequence according to any one of claims 1 to 6.

36. A method for representing a nucleic acid which comprises a sequence which can be detected in thyroid tumours or goitres, in which there is a translocation with a break point in the chromosomal band 19q13, wherein the sequence lies within the chromosomal band 19q13, characterised in that the method comprises the following steps:
a) Providing at least one of the primers according to claim 34 for carrying out a polymerase chain reaction,
b) Providing a nucleic acid sequence taken from the band 19q13 of the human chromosome 19 or a nucleic acid according to any one of claims 1 to 6,
c) Mixing the nucleic acid sequence or the nucleic acid with the primers,
d) Carrying out a polymerase chain reaction.

37. A pharmaceutical composition characterised in that it comprises:
at least one agent selected from the group comprising a nucleic acid, a vector, a polypeptide, a cell, an antibody, an antisense nucleic acid, RNAi, a ribozyme, each according to one of the preceding claims, and a combination thereof, and
at least one pharmaceutically acceptable carrier.

38. A method for the treatment and/or prevention of tumours and hyperplasias, characterised in that a compound is administered to a patient which prevents the effects of modified expression of the nucleic acids according to any one of the preceding claims.

39. Use of a compound which prevents the effects of modified expression of the nucleic acids according to any one of the preceding claims, for the manufacture of a medicament.

40. The use according to claim 39, characterised in that the medicament is for the treatment and/or prevention of tumours and/or hyperplasias, especially of tumours and/or hyperplasias of the thyroid.

41. Use of a nucleic acid having a sequence, wherein the sequence is selected from the group comprising SEQ. ID. No. 1, 2, 7, 10, 11 and 12, or derivatives thereof and/or polypeptides encoded thereby or derivatives thereof for the manufacture of a medicament, especially for the treatment of functional disorders of the thyroid and/or hyperplasias of the thyroid and/or thyroid tumours and/or for the manufacture of a diagnostic means, especially for the diagnosis of functional disorders of the thyroid and/or hyperplasias of the thyroid and/or thyroid tumours.

42. The use according to claim 41, characterised in that the polypeptide has an amino acid sequence in accordance with SEQ. ID. No. 13 and/or SEQ. ID. No. 15.

43. The use according to claim 42 or 43, characterised in that the nucleic acid would hybridise with the nucleic acid according to one of the sequences SEQ. ID. No. 1, 2, 7, 10, 11 and 12 without the degeneracy of the genetic code.

44. Use of a polypeptide having a sequence, wherein the sequence is selected from the group comprising SEQ. ID. No. 13 and SEQ. ID. No. 15, or derivatives thereof for the manufacture of a medicament, especially for the treatment of functional disorders of the thyroid and/or hyperplasias of the thyroid and/or thyroid tumours and/or for the manufacture of a diagnostic means, especially for the diagnosis of functional disorders of the thyroid and/or hyperplasias of the thyroid and/or thyroid tumours.

45. A method for screening a means for the treatment of functional disorders of the thyroid and/or hyperplasias of the thyroid and/or thyroid tumours and/or a diagnostic means for the diagnosis of functional disorders of the thyroid and/or hyperplasias of the thyroid and/or thyroid tumours, comprising the steps:
a) Providing a candidate compound,
b) Providing an expression system and/or activity system;
c) Bringing the candidate compound in contact with the expression system and/or the activity system;
d) Determining whether under the influence of the candidate compound, the expression and/or the activity of a nucleic acid having a sequence, wherein the sequence is selected from the group comprising SEQ. ID. No. 1, 2, 7, 10, 11 and 12, or derivatives thereof and/or polypeptides encoded thereby and/or polypeptides having a sequence in accordance with SEQ. ID. No. 13 or 15 or derivatives thereof, is modified.

46. The method according to claim 45, characterised in that the candidate compound is contained in a compound library.

47. The method according to claim 45 or 46, characterised in that the candidate compound is selected from the group of compound classes comprising peptides, proteins, antibodies, anticalins, functional nucleic acids and small molecules.

48. The method according to claim 47, characterised in that the functional nucleic acids are selected from the group comprising aptamers, aptazymes, ribozymes, spiegelmers, antisense oligonucleotides and RNAi.

49. Use of a nucleic acid having a sequence, wherein the sequence is selected from the group comprising SEQ. ID. No. 1, 2, 7, 10, 11 and 12, or derivatives thereof and/or polypeptides encoded thereby or derivatives thereof and/or a polypeptide having a sequence in accordance with SEQ. ID. No. 13 or 15 or a derivative thereof and/or an especially natural interaction partner of a nucleic acid having a sequence wherein the sequence is selected from the group comprising SEQ. ID. No. 1, 2, 7, 10, 11 and 12, or derivatives thereof and/or polypeptides encoded thereby or derivatives thereof and/or a nucleic acid coding therefor and/or an interaction partner of a polypeptide having a sequence in accordance with SEQ. ID. No. 13 or 15 or a derivative thereof as a target molecule for the development and/or manufacture of a diagnostic means for the diagnosis of functional disorders of the thyroid and/or hyperplasias of the thyroid and/or thyroid tumours and/or for the development and/or manufacture of a medicament for the prevention and/or treatment of functional disorders of the thyroid and/or hyperplasias of the thyroid and/or thyroid tumours.

50. The use according to claim 49, characterised in that the medicament or the diagnostic means comprises an agent which is selected from the group comprising antibodies, peptides, anticalins, small molecules, antisense molecules, aptamers, spiegelmers and RNAi molecules.

51. The use according to claim 50, characterised in that the agent interacts with a polypeptide having a sequence in accordance with SEQ. ID. No. 13 or 15 or a derivative thereof or an interaction partner thereof.

52. The use according to claim 50, characterised in that the agent interacts with a nucleic acid having a sequence, wherein the sequence is selected from the group comprising SEQ. ID. No. 1, 2, 7, 10, 11 and 12, or derivatives thereof and/or with a nucleic acid coding for an especially natural interaction partner, especially with mRNA, genomic nucleic acid or cDNA.

53. Use of a polypeptide that interacts with a peptide which is encoded by a nucleic acid having a sequence wherein the sequence is selected from the group comprising SEQ. ID. No. 1, 2, 7, 10, 11 and 12, or derivatives thereof and/or with a polypeptide in accordance with SEQ. ID. No. 13 or 15 and/or interacts with an especially natural interaction partner thereof for the development or manufacture of a diagnostic means for the diagnosis of functional disorders of the thyroid and/or hyperplasias of the thyroid and/or thyroid tumours and/or for the development or manufacture of a medicament for the prevention and/or treatment of functional disorders of the thyroid and/or hyperplasias of the thyroid and/or thyroid tumours.

54. The use according to claim 53, characterised in that the polypeptide is selected from the group comprising antibodies and binding polypeptides.

55. Use of a nucleic acid that interacts with a polypeptide wherein the polypeptide is encoded by a nucleic acid having a sequence wherein the sequence is selected from the group comprising SEQ. ID. No. 1, 2, 7, 10, 11 and 12, or derivatives thereof and/or with a polypeptide in accordance with SEQ. ID. No. 13 or 15 and/or with an especially natural interaction partner thereof for the development or manufacture of a diagnostic means for the diagnosis of functional disorders of the thyroid and/or hyperplasias of the thyroid and/or thyroid tumours and/or for the development or manufacture of a medicament for the prevention and/or treatment of functional disorders of the thyroid and/or hyperplasias of the thyroid and/or thyroid tumours.

56. The use according to claim 55, characterised in that the nucleic acid is selected from the group comprising aptamers and spiegelmers.

57. Use of a first nucleic acid that interacts with a second nucleic acid wherein the second nucleic acid has a sequence wherein the sequence is selected from the group comprising SEQ. ID. No. 1, 2, 7, 10, 11 and 12 or derivatives thereof and/or with a nucleic acid which codes for an interaction partner of a polypeptide having a sequence in accordance with SEQ. ID. No. 13 or 15 for the development or manufacture of a medicament for the prevention and/or treatment of functional disorders of the thyroid and/or hyperplasias of the thyroid and/or thyroid tumours.

58. The use according to claim 57, characterised in that the interacting first nucleic acid is an antisense oligonucleotide, a ribozyme and/or RNAi.

59. The use according to claim 57 or claim 58, characterised in that the second nucleic acid is the respective cDNA or mRNA.

60. A pharmaceutical composition comprising at least one agent selected from the group as defined by the use according to any one of claims 49 to 59, and at least one pharmaceutically acceptable carrier, especially for the prevention and/or treatment of functional disorders of the thyroid and/or hyperplasias of the thyroid and/or thyroid tumours.

61. A kit for characterising the state of the thyroid comprising at least one agent that is defined by the use according to any one of claims 49 to 59.


SEQUENCE LISTING (43 sequences)

SEQ ID NO: 1 - DNA, 394 bp, Homo sapiens
  misc_feature: EST, Acc. No. BE 564764
ccaaagcagaatccccagcatgtgttcctgaagcgtccagcactccggcgagtctgtggagccctgtcagcaccatccccaccccgagatggggccgcatcgtctatggatgtgcctttctgcttgttgtcctgctgtccatgctatgcctggtattggcccattggcccaaacgcctgttctctggagagctgatctacatcgcagcggctgtgttgctgctggtgctcattgtgggattcactttcaccgtctggagacagccccagagcaacactcctctttacttcaaggtccccctgttgcctgttctcctctggtcagcatctctgcacatgtttacctgatgatgcagatgaccactgagacctgggcccaatgtggagtctggaat

SEQ ID NO: 2 - DNA, 394 bp, Homo sapiens; identical to SEQ ID NO: 1
  misc_feature: EST, Acc. No. BF 029168

SEQ ID NO: 3 - DNA, 867 bp, Homo sapiens
  misc_feature: EST, Acc. No. AF 201937
tgcaggagttttccgggagcgcgtggggcttgaggccgagaacggcccttgctgccaccaacatggagactttgtaccgtgtcccgttcttagtgctcgaatgtcccaacctgaagctgaagaagccgccctggttgcacatgccgtcggccatgactgtgtatgctctggtggtggtgtcttacttcctcatcaccggaggaataatttatgatgttattgttgaacctccaagtgtcggttctatgactgatgaacatgggcatcagaggccagtagctttcttggcctacagagtaaatggacaatatattatggaaggacttgcatccagcttcctatttacaatgggaggtttaggtttcataatcctggaccgatcgaatgcaccaaatatcccaaaactcaatagattccttcttctgttcattggattcgtctgtgtcctattgagttttttcatggctagagtattcatgagaatgaaactgccgggctatctgatgggttagagtgcctttgagaagaaatcagtggatacctggatttgctcctgtcaatgaagttttaaaggctgtaccaatcctctaatatgaaatgtggaaaagaatgaatgaagcagcagcagtaaaagaaatatctagtgaaaaaacaggaagcgtattgaagcttggactagaatttcttcttgggtattaaagagacaagtttatcacagaattttttttcctgctggccctattgctataccaatgatgttgagtggcattttctttttagtttttcattaaaatatattccatatctacaactataatatcaaataaagtgttattttttacaaccccctaacattttttggggtgg

SEQ ID NO: 4 - DNA, 535 bp, Homo sapiens
  misc_feature: EST, Acc.No. BE 271492
tagactttgtaccgtgtcccgttcttagtgctcgaatgtcccaacctgaagctgaagaagccgccctggttgcacatgccgtcggccatgactgtgtatgctctggtggtggtgtcttacttcctcatcaccggaggaataatttatgatgttattgttgaacctccaagtgtcggttctatgactgatgaacatgggcatcagaggccagtagctttcttggcctacagagtaaatggacaatatattatggaaggacttgcatccagcttcctatttacaatgggaggtttaggtttcataatcctggaccgatcgaatgcaccaaatatcccaaaactcaatagattccttcttctgttcattggattcgtctgtgtcctattgagttttttcatggctagagtattcatgagaatgaaactgccgggctatctgatgggttagagtgcctttgagaagaaatcagtggatactggatttgctcctgtcaatgaagttttaaaggctgtaccaatcctctaatatgaaatgt

SEQ ID NO: 5 - DNA, 2506 bp, Homo sapiens
  misc_feature: EST, Acc. No. AF 345 987
ccttgcggagccggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatggctggtctgggccccggcgtaggcgattcagaggggggaccccggcccctgttctgcagaaagggggctctgaggcagaaggtggtccacgaagtcaagagccacaagttcaccgctcgcttcttcaagcagcccaccttctgcagccactgcaccgacttcatctggggtatcggaaagcagggcctgcaatgtcaagtctgcagctttgtggttcatcgacgatgccacgaatttgtgaccttcgagtgtccaggcgctgggaagggcccccagacggacgacccccggaacaaacacaagttccgcctgcatagctacagcagccccaccttctgcgaccactgtggctccctcctctacgggcttgtgcaccagggcatgaaatgttcttgctgcgagatgaacgtgcaccggcgctgtgtgcgtagcgtgccctctctgtgcggtgtggaccacaccgagcgccgcgggcgcctgcagctggagatccgggctcccacagcagatgagatccacgtaactgttggcgaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttcaacctgaagccaggggatgtggagcgccggctcagcgtggaggtgtgggactgggaccggacctcccgcaacgacttcatgggggccatgtcctttggcgtctcggagctgctcaaggcgcccgtggatggctggtacaagttactgaaccaggaggagggcgagtattacaatgtgccggtggccgatgctgacaactgcagcctcctccagaagtttgaggcttgtaactaccccctggaattgtatgagcgggtgcggatgggcccctcttcctctcccatcccctccccttcccctagtcccaccgaccccaagcgctgcttcttcggggcgagtccaggacgcctgcacatctccgacttcagcttcctcatggttctaggaaaaggcagttttgggaaggtgatgctggccgagcgcaggggctctgatgagctctacgccatcaagatcttgaaaaaggacgtgatcgtccaggacgacgatgtggactgcacgctggtggagaaacgtgtgctggcgctggggggccggggtcctggcggccggccccacttcctcacccagctccactccaccttccagaccccggaccgcctgtatttcgtgatggagtacgtcaccgggggagacttgatgtaccacattcaacagctgggcaagtttaaggagccccatgcagcgttctacgcggcagaaatcgctatcggcctcttcttccttcacaatcagggcatcatctacagggacctgaagctggacaatgtgatgctggatgctgagggacacatcaagatcactgactttggcatgtgtaaggagaacgtcttccccgggacgacaacccgcaccttctgcgggaccccggactacatagccccggagatcattgcctaccagccctatgggaagtctgtcgattggtggtcctttggagttctgctgtatgagatgttggcaggacagcctcccttcgatggggaggacgaggaggagctgtttcaggccatcatggaacaaactgtcacctaccccaagtcgctttcccgggaagccgtggccatctgcaaggggttcctgaccaagcacccagggaagcgcctgggctcagggcctgatggggaacctaccatccgtgcacatggctttttccgctggattgactgggagcggctggaacgattggagatcccgcctcctttcagaccccgcccgtgtggccgcagcggcgagaactttgacaagttcttcacgcgggcggcgccagcgctgacccctccagaccgcctagtcctggccagcatcgaccaggccgatttccagggcttcacctacgtgaaccccgacttcgtgcacccggatgcccgcagccccaccagcccagtgcctgtgcccgtcatgtaatctcacccgccgccactaggtgtccccaacgtcccctccgccgtgccggcggcagccccacttcacccccaacttcaccaccccctgtcccattctagatcctgcaccccagcattccagctctgcccccgcgggttctagacgcccctcccaagcgttcctggccttctgaactccatacagcctctacagccgtcccgcgttcaagacttgagcg

SEQ ID NO: 6 - DNA, 1149 bp, Homo sapiens
  misc_feature: EST, Acc. No. X 625337
ctcccagagaggagacagagacggactcccaaagagtcaagttgcagagggagggggagacagagtccgcctgagagagaatagagttcacagagccagagagacaaagacagggaaagaaacaaactcagggagacagaggcggaagaatttatcagacgtagagggagagacagagggagactgattcacagtgaggcagaccaagaccccgagaagaaccacgacaccaagacccagagatggcgacagatacagatggagacacacagcagaggaaatagagaaatagaaagaaatagaaacagcatttctcggagacagggaaaagaaaacagatccactgagaggcagaaacagagacacatcgagaaagctcgctccaggaatagagggagagggacagaggtcacaagaaagacacacgcaggcagagagctacacaaataatggagaggccaggggaggaaataaagactcagccggcatcggagaaagtgagaaccctagcgccctgcctgtccactgctggacccctagcgtggagcataaagtttgttgaaggaaggagaggggcagggtcagacacaggaccccagggcgcccacaggacacacgaggcacctagtgggggaggaacgcgggcaggatgacagattgcagggtggtgggggggagccaggctcagaggatgcccctccctccagccaggcccccggagtgggtgtgtgcacgtgtggggggcgggcagggaggacatttgtcccgtgtctccgggaggggagcgcctttaagccgaaacccgccctctcggtcgtcctgcaacgcctcccccacccggggctcccacatttcagcaggtgccggagctggagctcccaccgccgccgcccgtgcctccggctgccggcgcccctgcctttggctcttcctccccactcgcccgctccccctggcggagccggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatg

SEQ ID NO: 7 - DNA, 647 bp, Homo sapiens
  misc_feature: cDNA sequence of the CAT homologous gene on 19q13.4
catgtgttcctgaagcgtccagcactccggcgagtctgtggagccctgtcagcaccatccccaccccgagatggggccgcatcgtctatggatgtgcctttctgcttgttgtcctgctgtccatgctatgcctggtattggcccattggcccaaacgcctgttctctggagagctgatctacatcgcagcggctgtgttgctgctggtgctcattgtgggattcactttcaccgtctggagacagccccagagcaacactcctctttacttcaaggtccccctgttgcctgttctcctctggtcagcatctctgcacatgtttacctgatgatgcagatgaccactgagacctgggcccaatgtggagtctggatgttgattggtttgctatgtattttggatatgggatcccacacagcttggagaacaatcaacagccaccagcgtcaagctcccaaactcctcatgaaaacatccctagtcccttgctgaattagtttaaccacggaaaagggatggtccagtccactggaggtgtcctctggatgaagggaaagtgtgaagctctgtggagcatgaaggtgggatacacttaaagccccttgtgcattgccagtgagcaaacactttttaaaaaactttattt

SEQ ID NO: 8 - DNA, 749 bp, Homo sapiens
  misc_feature: cDNA sequence of the DC2 homologous gene on 19q13.4 (our sequence)
tagaggccgagaatggcccttgctgccaccaacatggagactttgtaccgtgtcccgttcttagcgcttgaatgtcccaacctgaagctgaagaagccgccctggctgcacatgccgttggccatgactatgtatgctctggtggtggtgtcttacttcctcatcaccagaggaatcgtttatgatgttacggttgaaccgccaggtgttggctctatgactgatgaacaagggcatcagaggccagtagctttcttggcctacagagtaagtggacaatattattatggaaggacttggatccagcttcctgtttacaatgggaggtttaggtttcataatcctggaccgatcgaatgcaccaaatatcccaaaactcaatagatttcttcttctattcgttggattcgtctgtgtcctattgagtttttttcacggctagagtattcgtgagaatgaaactaccgggctctctgatgggttagagtgcctttaagaagaaatcagtgcatactggatttgctcctgtcaatgaagttttaaaggctgtccaatcctctaatatgagatgtagaaaagaaggaagagcagcagtaaaagaaatatctagtgaaaaaccaggaagtgtattgaagcttggactagaatttcttctttattaaagagacaaatttatcacagtattttcttttcctgctgaccacattgctataccaatgatgatgagtggcattttcttcttagttttt

SEQ ID NO: 9 - DNA, 3457 bp, Homo sapiens
  misc_feature: cDNA sequence of the PKCgamma homologous gene on 19q13.4, almost 100% homology as far as comparable, composed of Acc. No. X 625337 and AF 345987
ctcccagagaggagacagagacggactcccaaagagtcaagttgcagagggagggggagacagagtccgcctgagagagaatagagttcacagagccagagagacaaagacagggaaagaaacaaactcagggagacagaggcggaagaatttatcagacgtagagggagagacagagggagactgattcacagtgaggcagaccaagaccccgagaagaaccacgacaccaagacccagagatggcgacagatacagatggagacacacagcagaggaaatagagaaatagaaagaaatagaaacagcatttctcggagacagggaaaagaaaacagatccactgagaggcagaaacagagacacatcgagaaagctcgctccaggaatagagggagagggacagaggtcacaagaaagacacacgcaggcagagagctacacaaataatggagaggccaggggaggaaataaagactcagccggcatcggagaaagtgagaaccctagcgccctgcctgtccactgctggacccctagcgtggagcataaagtttgttgaaggaaggagaggggcagggtcagacacaggaccccagggcgcccacaggacacacgaggcacctagtgggggaggaacgcgggcaggatgacagattgcagggtggtgggggggagccaggctcagaggatgcccctccctccagccaggcccccggagtgggtgtgtgcacgtgtggggggcgggcagggaggacatttgtcccgtgtctccgggaggggagcgcctttaagccgaaacccgccctctcggtcgtcctgcaacgcctcccccacccggggctcccacatttcagcaggtgccggagctggagctcccaccgccgccgcccgtgcctccggctgccggcgcccctgcctttggctcttcctccccactcgcccgctccccctkgcggagccggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatggctggtctgggccccggcgtaggcgattcagaggggggaccccggcccctgttctgcagaaagggggctctgaggcagaaggtggtccacgaagtcaagagccacaagttcaccgctcgcttcttcaagcagcccaccttctgcagccactgcaccgacttcatctggggtatcggaaagcagggcctgcaatgtcaagtctgcagctttgtggttcatcgacgatgccacgaatttgtgaccttcgagtgtccaggcgctgggaagggcccccagacggacgacccccggaacaaacacaagttccgcctgcatagctacagcagccccaccttctgcgaccactgtggctccctcctctacgggcttgtgcaccagggcatgaaatgttcttgctgcgagatgaacgtgcaccggcgctgtgtgcgtagcgtgccctctctgtgcggtgtggaccacaccgagcgccgcgggcgcctgcagctggagatccgggctcccacagcagatgagatccacgtaactgttggcgaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttcaacctgaagccaggggatgtggagcgccggctcagcgtggaggtgtgggactgggaccggacctcccgcaacgacttcatgggggccatgtcctttggcgtctcggagctgctcaaggcgcccgtggatggctggtacaagttactgaaccaggaggagggcgagtattacaatgtgccggtggcygatgctgacaactgcagcctcctccagaagtttgaggcttgtaactaccccctggaattgtatgagcgggtgcggatgggcccctcttcctctcccatcccctccccttcccctagtcccaccgaccccaagcgctgcttcttcggggcgagtccaggacgcctgcacatctccgacttcagcttcctcatggttctaggaaaaggcagttttgggaaggtgatgctggccgagcgcaggggctctgatgagctctacgccatcaagatcttgaaaaaggacgtgatcgtccaggacgacgatgtggactgcacgctggtggagaaacgtgtgctggcgctggggggccggggtcctggcggccggccccacttcctcacccagctccactccaccttccagaccccggaccgcctgtatttcgtgatggagtacgtcaccgggggagacttgatgtaccacattcaacagctgggcaagtttaaggagccccatgcagcgttctacgcggcagaaatcgctatcggcctcttcttccttcacaatcagggcatcatctacagggacctgaagctggacaatgtgatgctggatgctgagggacacatcaagatcactgactttggcatgtgtaaggagaacgtcttccccgggacgacaacccgcaccttctgcgggaccccggactacatagccccggagatcattgcctaccagccctatgggaagtctgtcgattggtggtcctttggagttctgctgtatgagatgttggcaggacagcctcccttcgatggggaggacgaggaggagctgtttcaggccatcatggaacaaactgtcacctaccccaagtcgctttcccgggaagccgtggccatctgcaaggggttcctgaccaagcacccagggaagcgcctgggctcagggcctgatggggaacctaccatccgtgcacatggctttttccgctggattgactgggagcggctggaacgattggagatcccgcctcctttcagaccccgcccgtgtggccgcagcggcgagaactttgacaagttcttcacgcgggcggcgccagcgctgacccctccagaccgcctagtcctggccagcatcgaccaggccgatttccagggcttcacctacgtgaaccccgacttcgtgcacccggatgcccgcagccccaccagcccagtgcctgtgcccgtcatgtaatctcacccgccgccactaggtgtccccaacgtcccctccgccgtgccggcggcagccccacttcacccccaacttcaccaccccctgtcccattctagatcctgcaccccagcattccagctctgcccccgcgggttctagacgcccctcccaagcgttcctggccttctgaactccatacagcctctacagccgtcccgcgttcaagacttgagcg

SEQ ID NO: 10 - DNA, 1437 bp, Homo sapiens
  misc_feature: cDNA of CAT-A
tggatatgggtagatgaaacctcggatacgtgggagctaccgtgggtgtattaggctctctgatgttggatgaacagggacctgggttaatcttgccctactctctctaattcaggtaccagccagacgagaatttcagcaagaacgagaagccgaaggaggaagtagttgagatgaattctgtacccaaagcagaatccccagcatgtgttcctgaagcgtccagcactccggcgagtctgtggagccctgtcagcaccatccccaccccgagatggggccgcatcgtctatggatgtgcctttctgcttgttgtcctgctgtccatgctatgcctggtattggcccattggcccaaacgcctgttctctggagagctgatctacatcgcagcggctgtgttgctgctggtgctcattgtgggattcactttcaccgtctggagacagccccagagcaacactcctctttacttcaaggtccccctgttgcctgttctcctctggtcagcatctctgcacatgtttacctgatgatgcagatgaccactgagacctgggcccaatgtggagtctggatgttgattggatttgctatgtattttggatatgggatcccacacagcttggagaacaatcaacagccaccagcgtcaagctcccaaactcctcatgaaaacatccctagtcccttgctgaattagtttaaccacggaaaagggatggtccagtccactggaggtgtcctctggatgaagggaaagtgtgaagctctgtggagcatgaaggtgggatacacttaaagccccttgtgcattgccagtgagcaaacactttttaaaaaactttattttgttgtttgtttgtttgtttgtttgtttgtttgttatagaggtgaggacttgctctgttgcccaggcggaacttgaactcctgggctcaagtgatcctcctgcctcagtcttccaagcagctgggactacaggtaccccatcatacccagcttaacaaaatatctttatttgatttgattttttttctttttgagacagagtctcgctctattgcccagtctggagtgcagtggcatgatcttggctcactgcaacctccgcctcccgggttcaagtgattcccctgygtgagcctccagagtagctggaattacaggcacatgacactgtgcccagctaatttttctttattagyagagaccaggtttcaccatgttggccaggctggtcttgaactcctgacctcaagtgatccacctgcctcggcctcccaaagtgctgagattacaggcctgagccaccgtgcccagccaaaataactttaaaaacttacatttttaaaaattgacaaayaaaagttgtctatgtttatggtgtacaattgcatatattatatgtattaaaattgtgtatgtttatggtat

SEQ ID NO: 11 - DNA, 1168 bp, Homo sapiens
  misc_feature: cDNA of CAT-A1
tggatatgggtagatgaaacctcggatacgtgggagctaccgtgggtgtattaggctctctgatgttggatgaacagggacctgggttaatcttgccctactctctctaattcaggtaccagccagacgagaatttcagcaagaacgagaagccgaaggaggaagtagttgagatgaattctgtacccaaagcagaatccccagcatgtgttcctgaagcgtccagcactccggcgagtctgtggagccctgtcagcaccatccccaccccgagatggggccgcatcgtctatggatgtgcctttctgcttgttgtcctgctgtccatgctatgcctggtattggcccattggcccaaacgcctgttctctggagagctgatctacatcgcagcggctgtgttgctgctggtgctcattgtgggattcactttcaccgtctggagacagccccagagcaacactcctctttacttcaaggtccccctgttgcctgttctcctctggtcagcatctctgcacatgtttacctgatgatgcagatgaccactgagacctgggcccaatgtggagtctggatgttgattggatttgctatgtattttggatatgggatcccacacagcttggagaacaatcaacagccaccagcgtcaagctcccaaactcctcatgaaaacatccctagtcccttgctgaattagtttaaccacggaaaagggatggtccagtccactggaggtgtcctctggatgaagggaaagtgtgaagctctgtggagcatgaagagatggagtttcaccttgttgccgaggctggtcttgaactcctgagctgaagccatcctcctgcctctggcctctaaaagtgctgggattccaggcgtgagccagcgtgcccggccatcatgtttacttcaaacagattttctctggcgtcttctaggacacttgtatctgatctttactgaaagtcacattcttagcaagacttaaccttacatcctgagtaaaattgcgaattccacagatgccctcaatctttcctgacctcggctcctcttctgtttcccggcatggcttgaagcgcaacggcattctacctgtttactcaggatgtgtcttaccattaaagtccctgcctgcagagcagttggcttgatttcacct

SEQ ID NO: 12 - DNA, 5278 bp, Homo sapiens
  exon (1)..(312): Exon 1 of CAT-A/A1
tggatatgggtagatgaaacctcggatacgtgggagctaccgtgggtgtattaggctctctgatgttggatgaacagggacctgggttaatcttgccctactctctctaattcaggtaccagccagacgagaatttcagcaagaacgagaagccgaaggaggaagtagttgagatgaattctgtacccaaagcagaatccccagcatgtgttcctgaagcgtccagcactccggcgagtctgtggagccctgtcagcaccatccccaccccgagatggggccgcatcgtctatggatgtgcctttctgcttggtgagcagtggcgtttctccgcggggtgttttgaacctgatagcatgcggcagggggccgagtagggagtgtggggctttggtagttgagaggagttgtgaagatgtggtggcccttcttagtaggcgcacttttggccgggcacagtggattacacatggaatcccagcactttgggaggctgaggcgggacaactgcttgagcctgggcagtcgaggcttcagtgaatcaagaccacaccactgcactgtagtctgggcaatggggcgagatcccatctcaaaaaacaaacaaaaagccatatatatatatacacacacacacacatatatacacacacacacacatacatatacacacacaaatatatatatataaaattaaaagtttaggctgggtgtggtggctcactttagaaggctgaggcaagtggatcacttgaggtgaggagttgagaccagcctggccaacatggtgaaaccccatctctactaaaaatacaaaaaattaggtcaggcacagtggctcacgcctgtaatcccagcactctgggaggccgggtgccgggggggagtggatcacaaggtcaggagttcgagatcagcctggccaacatggtgaaaccccatctctactaaaaatacaaaaattagccgagcacggtggcagacgcctgtaatcccagctactcggggagactgaggcaggagaattatttgaaccggagaggccgagtttccagtgagccaagatcacgccactgcacttcagcctgggcaacagagaaagactttttctcaaaaaaaaaaaaaaaatacaaaaaattagctgggtgtggtggcagttacctgtaatcccagctacttgggaggctgaggcaggagaatcacttgaacccaggaggcagaggttgcagtgagccaagattgtgccactgcactccatcctgggcaacaagagtgaaactgcatctcaaaaaaaaaaaaaaagtatttgggtgtgctgacacgtgcctgtagtccctacttgggaggctggcacacacacacacacacacacacacacacacacacacacacacacccatacccatatatatggtttgggggataggatgtggcccgagatcttcacttatttagcttctgaagttgaacctgttctccccaaccttgacccttttccagttgtcctgctgtccatgctatgcctggtattggcccattggcccaaacgcctgttctctggagagctgatctacatcgcagcggctgtgttgctgctggtgctcattgtgggattcactttcaccgtctggagacagccccagagcaacactcctctttacttcaaggtaggtggccttatgtccccacaccaccctcctgagtcagtggactctgcatgcctcaagctctaaatactctacgggaccaggggtaaaaagggcagttgcgaaaaaccaatggactgttcatggacaccccctccagcccccacatatcagtacttcccaaaacctgtctcttccagaacacccaatggaatacctactcagaacaggcattgagccaggtgaggtggctcacacctgtaatcccagcactttgggaggccaaggcgggtggatcacttgaggcgaggagttcgggaccagcctgaccaacatggtgaaattccggttctactaaaaatacaaaaattagccgggcatggtggtgtgtgccggtaatcccagctactcaggaggctgaggcaggagaatcgcttgaacccaggaggcggatgttgcattcagctgagatcgcgccactgcactccagcctgggtgacacagcaagactctgtctcaaaaaaaaaaaaaaaagaataggcattgaatatgcgactgcaacagagctgaggccttcccaccccttggaataagggtcccttgcagacgttcgtggtgtgctcacggttaacgctttgcctgcaggtccccctgttgcctgttctcctctggtcagcatctctgcacatgtttacctgatgatgcagatgaccactgagacctgggcccaatgtggagtctggatgttgattggtgagtggcttcctggaaaaaaagaggcctcttgggtgtctgagcccaacactcttcctgcgacctttccctatagctgtctcaggctaacagtgtggggtgtgcctgtagtcccagctactcaggagatgaaggcaggagcatcgcttgagaccaggagctccagaccagcctgggcaacatggcgagactccacctctaccaaaataaaaaactggccgcacgcagtggctcgcacctataatcccagcacttcagggggccaaggcaggtgggtcacctaagatcaggagctcaagaacagcctggccaacatggtgaaaccccatctctactaaaaatacaaaaaagtagccgggcgtggtggcgggcgcctgtaatcccacctactcaggaggctgaggcatgagaattgcttgaacccaggaggcagagtttgcggtgagccgagatcatgccattgcactccagcgcaggcgacaggagctgagattgtgccactgcactccagcctgggtgacagagtgagatgctgtctcaaaaaaaaaaaaaaaaaaaaaaaaaaattagccaggcacggtggcacctgcctgtagtcacatctattctgaaggctgaggtgggaggatcgcttgaacccaggaggtcgaggctgcagtgagccatgatcatgccactgcactccagcctgggcaatggagcgaagctctatttctaaataaaagaaacaagaaaacctccaaataaacaaaccacagtataatcgctatagggcatttataagggaaggggtgactacatttcctttcattgtctcatttccctgctaggatttgctatgtattttggatatgggatcccacacagcttggagaacaatcaacagccaccagcgtcaagctcccaaactcctcatgaaaacatccctagtcccttgctgaattagtttaaccacggaaaagggatggtccagtccactggaggtgtcctctggatgaagggaaagtgtgaagctctgtggagcatgaaggtgggatacacttaaagccccttgtgcattgccagtgagcaaacactttttaaaaaactttattttgttgtttgtttgtttgtttgtttgtttgtttgttatagaggtgaggacttgctctgttgcccaggcggaacttgaactcctgggctcaagtgatcctcctgcctcagtcttccaagcagctgggactacaggtaccccatcatacccagcttaacaaaatatctttatttgatttgattttttttctttttgagacagagtctcgctctattgcccagtctggagtgcagtggcatgatcttggctcactgcaacctccgcctcccgggttcaagtgattcccctgygtgagcctccagagtagctggaattacaggcacatgacactgtgcccagctaatttttctttattagyagagaccaggtttcaccatgttggccaggctggtcttgaactcctgacctcaagtgatccacctgcctcggcctcccaaagtgctgagattacaggcctgagccaccgtgcccagccaaaataactttaaaaacttacatttttaaaaattgacaaayaaaagttgtctatgtttatggtgtacaattgcatatattatatgtattaaaattgtgtatgtttatggtatacatgaagcttggaaatatgtatacattgtggaattgctaaatcaggttgattaacgtattcattaccttacataccttttttttttttgagatggagtctccctctgtcgcccagactggagtgcagtagtgtgatctcagctcactgcaacctccgcctcccaggttcaagcaattctcctgcctcagcctcctgagtagctgggattacaggtgtgccccaccattcccggctaatttttgtgtttttagtagagacggggtttcaccatgtcggtcaggctggtctcgaatgcctgacctcatgatctgcccgcctcggcctcccaaagtgctgggattacaggcgtgagccaccacacccagccttacgtgccatttttttttttttttgtggtaaaaacatttaaaatggccgggtgcggtggctcatgcctgtaatcccagcactttgggaggccaaggcaggtggatcatctgaggtcaggagttcaagaccagtctggccaacgtggcgaaaccctgtctctactaaaaatacaaaaaattagctggggcgcagtggcaggtgcctgtaatcccagctactcgggagactgaggcttgaacccagaggcagaggttgcagtgagctgagattgcaccattgcactccagcttgggcaacaagagtgaaattcatctcaaaaaaaaaaaaaaatagccaggtgcggtctggaactcccggcctcaagtgatcctcttgcctcagcctcccgagtagctgggaatacaggcatgcgctaccacaccagggtaatttttgtatttacagtagagatggagtttcaccttgttgccgaggctggtcttgaactcctgagctgaagccatcctcctgcctctggcctctaaaagtgctgggattccaggcgtgagccagcgtgcccggccatcatgtttacttcaaacagattttctctggcgtcttctaggacacttgtatctgatctttactgaaagtcacattcttagcaagacttaaccttacatcctgagtaaaattgcgaattccacagatgccctcaatctttcctgacctcggctcctcttctgtttcccggcatggcttgaagcgcaacggcattctacctgtttactcaggatgtgtcttaccattaaagtccctgcctgcagagcagttggcttgatttcacct

SEQ ID NO: 13 - protein, 142 aa, Homo sapiens
  MISC_FEATURE: protein sequence of ORF 1 of mRNA of CAT-A
MGPHRLWMCLSACCPAVHAMPGIGPLAQTPVLWRADLHRSGCVAAGAHCGIHFHRLETAPEQHSSLLQGPPVACSPLVSISAHVYLMMQMTTETWAQCGVWMLIGFAMYFGYGIPHSLENNQQPPASSSQTPHENIPSPLLN

SEQ ID NO: 14 - protein, 119 aa, Homo sapiens
  MISC_FEATURE: protein sequence of ORF 2 of mRNA of CAT-A
MNSVPKAESPACVPEASSTPASLWSPVSTIPTPRWGRIVYGCAFLLVVLLSMLCLVLAHWPKRLFSGELIYIAAAVLLLVLIVGFTFTVWRQPQSNTPLYFKVPLLPVLLWSASLHMFT

SEQ ID NO: 15 - protein, 140 aa, Homo sapiens
  MISC_FEATURE: protein sequence of ORF 3 of mRNA of CAT-A
FRYQPDENFSKNEKPKEEVVEMNSVPKAESPACVPEASSTPASLWSPVSTIPTPRWGRIVYGCAFLLVVLLSMLCLVLAHWPKRLFSGELIYIAAAVLLLVLIVGFTFTVWRQPQSNTPLYFKVPLLPVLLWSASLHMFT

SEQ ID NO: 16 - DNA, 1686 bp, Homo sapiens
  misc_feature: Genomic sequence of the DC2 homologous gene
ttttttaattagtatttatttctgttcctttcatacatgttattgttcaggcagcagtcttacatacctatctgaaataatctaccatcacacactctagttaaagccaaagctccgtaggtaaagctgaccagctgtccagagttctcagctcatggaatcttgcttcttgattttatgttaatttctgaaagtcagaaatgtcatctccaaaaagtgttaagggggttgtaaaaaataatcactatatttgctattatagttgtaggtatgcagtatattttcttttctttctttctttctttttttttttttgaaacagagtctcgctctgtcgcccaggctggagtgcagtggcagcgatctaggctcactgcagcctccgcctcccaggttcacgtgattctcctgcctcagccttccaagtagctgggattacaggtgccccgccaccaccacgcccggctaatttttgtatttttagtagagatggggtttcaacatggtggccaggctggtctggaactcctgacctcgtgatctgcccaccttggcctcccaaagtgctgggactacagaagtgagccaccgcgcctggcttgtattttcttaaagaaataaaaaactaagaagaaaatgccactcatcatcattggtatagcaatgtggtcagcaggaaaagaaaatactgtgataaatttgtctctttaataaagaagaaattctagtccaagcttcaatacacttcctggtttttcactagatatttcttttactgctgctcttccttcttttctacatctcatattagaggattggacagcctttaaaacttcattgacaggagcaaatccagtatgcactgatttctttttttttttcttttttttttttttggtgagacggagatttactcttgttgtccaggctggagtgcaatggtgcgatctcggctcaccgcatcctctgcctcccgggttcaagcaattctcctgcctcagcctcccgagtagctgggattacagacatgtgtcaccacgcctggctaattttgtatttgtagtagggatggggttttaccatgttgcccaggctgatctcgaactcctgacctcagataatccgctcgcctcggcctcccaaagtgctgggattgacaggcgtgagccactgcacccagcctgatttcttcttaaaggcactctaacccatcagagagcccggtagtttcattctcacgaatactctagccgtgaaaaaaactcaataggacacagacgaatccaacgaatagaagaagaaatctattgagttttgggatatttggtgcattcgatcggtccaggattatgaaacctaaacctcccattgtaaacaggaagctggatccaagtccttccataataatattgtccacttactctgtaggccaagaaagctactggcctctgatgcccttgttcatcagtcatagagccaacacctggcggttcaaccgtaacatcataaacgattcctctggtgatgaggaagtaagacaccaccaccagagcatacatagtcatggccaacggcatgtgcagccagggcggcttcttcagcttcaggttgggacattcaagcgctaagaacgggacacggtacaaagtctccatgttggtggcagcaagggccattctcggcctc

SEQ ID NO: 17 - DNA, 1035 bp, Homo sapiens
  misc_feature: Possible mRNA of DC2 homologous gene
gaggccgagaatggcccttgctgccaccaacatggagactttgtaccgtgtcccgttcttagcgcttgaatgtcccaacctgaagctgaagaagccgccctggctgcacatgccgttggccatgactatgtatgctctggtggtggtgtcttacttcctcatcaccagaggaatcgtttatgatgttacggttgaaccgccaggtgttggctctatgactgatgaacaagggcatcagaggccagtagctttcttggcctacagagtaagtggacaatattattatggaaggacttggatccagcttcctgtttacaatgggaggtttaggtttcataatcctggaccgatcgaatgcaccaaatatcccaaaactcaatagatttcttcttctattcgttggattcgtctgtgtcctattgagtttttttcacggctagagtattcgtgagaatgaaactaccgggctctctgatgggttagagtgcctttaagaagaaatcagtgcatactggatttgctcctgtcaatgaagttttaaaggctgtccaatcctctaatatgagatgtagaaaagaaggaagagcagcagtaaaagaaatatctagtgaaaaaccaggaagtgtattgaagcttggactagaatttcttctttattaaagagacaaatttatcacagtattttcttttcctgctgaccacattgctataccaatgatgatgagtggcattttcttcttagtttttaaagaaaatatactgcatacctacaactataatagcaaatatagtgattattttttacaacccccttaacactttttggagatgacatttctgactttcagaaattaacataaaatcaagaagcaagattccatgagctgagaactctggacagctggtcagctttacctacggagctttggctttaactagagtgtgtgatggtagattatttcagataggtatgtaagactgctgcctgaacaataacatgtatgaaaggaacagaaataaatactaattaaaaaa

SEQ ID NO: 18 - DNA, 24801 bp, Homo sapiens
  misc_feature: Genomic organisation of PKCG contained in BAC clone Acc.AC008440 (Homo sapiens chromosome 19 clone CTC-331H23, complete sequence)
ggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatggctggtctgggccccggcgtaggcgattcagaggggggaccccggcccctgttttgcagaaagggggccctgaggcagaaggtggtccacgaagtcaagagccacaagttcaccgctcgcttcttcaagcagcccaccttctgcagccactgcaccgacttcatctggtgagggaagggggctgggggactgggggacgaggggactaggggtgcagactcctatcacgccgacccctgtggaaggaagaaggagggggctgtagtcccgactcccaggttctaggatggccagggaacgctgggagcttcgactcctgggtttcagtgaggaggaggctggttcctggagtgctgggtccgagggaggaggaggctggaggacagaggtcctggagtcttgggtctgagggaggaagggcctggggggctgggagcctggattcctgggtctgaaggaggaagaaactgggggctgaactccagtctaagggaagaagggctgggggccaaaatttctgggttctagaaagaggaggtggccggggcttggacacctgggccctgcgggaggagggtcagagagcgcaggccccctgtggctcgcagaggttgggggtccaggtacccctttctgcactgacctaggatccctgactcttccaggggtatcggaaagcagggcctgcaatgtcaaggtaagagctggggaccggggctcctgggaccctcaggagggtggaggctggggccccacagctgaggctgcttgacacacgtgttctctggtccccagagaggcgcgggggagcccggggcggggggtgtggcagagacacagcctgtggtggggagggagctttgatggtggggccaccgcggaggtggtgctgggggcccctccctcggccggctccaggtggggacagatatttggagaatctgttgccatgggaacatggagatttggaaaaggggagctgaaggggggaagggggagggggctggagatgcaaagtcagagcccccccccaccccaggctgccgtcgccatgacaacaccagccgtcctaggcaggggcaggccgggtggggtcaccaatgggcgagtgggggccgggcggggccggcagttctggggggcgggagaggggggcgagtccttgagcaccagctgctactgctgagaacagagtgagtcagggtggggggcgggccggcagcagcgcccccagactcacttctgcccaagttgctgcttcctgggctgcgtctgaagatatttcggttttcgctctttgaatctgtctgcctctctcctggctttctgatctccgtccgtgggcctgtgtctgtttgtcaatgggatcctattttctttctctctttttccatctcccttccctgagctctgtgctctgtgtctctctgtaagtctctgcgtctctgtttctgactctgagcccatctcttgggtttctgtctcctgcttctctctctggcctccgattttctctctgttggactctctgtgttgagatccctctctttctggttttctcagtgtccgagttccgctctctctttccaattttctgtctgctggggtctcccgctggactaatccatgcctccgtctgtgtctctatgattttcatctatagtctgcagctttgtggttcatcgacgatgccacgaatttgtgaccttcgagtgtccaggcgctgggaagggcccccagacggacgtgagtgctcggacacctggttctcctcctcgggccgtgcccccgccctcaccccctcggcgtccgtcccaatttctcctgctatttttatggctgggaggggaggggggctggagagatagggggagctatctggcccagattccttgcccttggcctggaaagggggaatgcgagggggactgacaggctggggacacgggtggggcacagagaggaggccggggtgaggagactgaagatgggtgctgccgggggtgggctgtgatccaggggtgaagggatttaaaaattgagagctgaggggcacacggagaaaaatatcagtgcaggtgcggagatgccaacataagacagagggaatctcagggagaggaacagagacagaagaagacagagacctaggagagactgaagctgaggcagagagagagagagatggagcagagaaagaaccagggagaaagagaggaatttggaggcaccaaaagatggacagagaaactccaagagacggagacacagacacctggagaaagagactaaaatagaaaatggtggatacagacaacagcttaggagatgctgagaggagacccagggaagtcccacagtacacttgcacacatgtgcgtgcatgtacagatgcccctgtcatcacagatgtgcagcacgcagagacacacagcctctccccaccccctctctctccatcagaggctcacaagaccagtcaaccctgagtgcccattcccgttcccttttctgctttatctctgagcctcagtttcctcctctataaaatggggctgatgatctagcattgaccaacagaactgtttacagtgacggaagcattctgtacatgcactgtccaataggcagccaccagctccatgtagccagtgagcacttgaaacagggcgactgtggctgaggattagaaattccatttcgtgtcatttgaattggcttaaattttttttttttatttttattttttgagacggagtcgcgctctgttgcccaagctggagtgcagtggcgcagtctcagctcaatgcaacttccgtttcccaggttcaagcaattcttctacctcagcctcctgagtagctgggactacaggcgcatgccaccacaccccgctaattttttttgtatttttagtagagatggggtttcaccatgttggccaggctggtctcgaactcctgaccttgtgatccacccgcctcggcctcccaaagtgttgggattacaggcgtgagccaccgcgcccggcagaactgacttaaatttaaacagccacatgtagttagtggccaccaaatgggacagcacacatctggacacttccaggcttgttttgaagtcaggtgggttcagagtttcgcccagggtttgacacaagatcggagacagttttatgatgtacagatggagagagaggcagagagagagagatccacagaagtccatgaggcgttttaccaccctctcagctgaaaataacagaagtctacatagaagatgtgacttcatagaacatatattgagcaccactgtctaccagcacgtgtatgtgattgatgacccccctcgtccactcacctccgccaccaacaccagataagtctgatgcatccagtgctcattgggtacactcatcaagatttttttttttgttttttgcctgtaatcccagctactcgggaggctgaggcagaattgcttgaacctgagcggcagaggttgcagtgagccaagatcacgccactgcactccagcctgggtgacagagcaagactttgtcttggaaaaaaaaaaaaagatttttttttgtttttgttgttttgtttgattttggggtattttttagatggagtttcactctgtcacccaggctggagtgcactggtgcaatcttggttcactgcaacctctacctcccaggttcaagcgattctcatgcctcagcctcccgagtagctgggactagaacaggcatgagccaccatggccggctaatttttatatttttagtagacagggtttcatcatgctggccaagctggtcttgctcctgacctcacgtgatccacccacctcggcctcccaaagtgctgggattacaggcgtgagccaccgcacccagccgatttttggggttttttgagacagggtcccactctgtcacctagactggagtacagtgatgggatcatagctcactgcagccttgaattctccaggctcaagtgctcctcctgccccagcttctcaagtagctgggactataggcacaagccacaacacctagctaattaaaaaaaaatgtttttgtagagatggagtctcactcactatattgcccaggctggtcttcaactcctggtctcactcgattctcctgcctcagcctcccaaaatgatgggattacaggcgtgagccactgcacctggcctcaagtattttgtatacagtataggttggatccacacaacagcttatttggttatttttccctgtctatctggtttgaatcccagctccaccactttttggttctgtgacatttcctgagttaatttacctctctgcacttgttgaattccttgtttgtaaagtggagatgataattatgctcactatggattgttttgaagatttagtgagtcagacatttgggatggtttctgacacatagcaagagccaaaatattattttttattcttgttaaaattattattatgaccaatgaggaaacgagtgaatagtgagaaggagatctttcctctgcatcactcgggggtttttttgtttttgtttttttttgctgttgagacagggtctcactctgttgcccaggctggagtgcaatagtgctatcactgctcactgcagtcttgacctccggggctcaagtgattcactgctggcagttgatcttcttaaaagtaacatgcaggccaggcacagtggctcacgcctgtaatcccaacactttgggagaccgaggcgggtggatcacctgaggttgggagttcgagaccagcctgaccaacatggagaagccacgtgtctactaagaatacaaaattagctgggcgtggtggcacacgcctgtaatcccagctactcaggaggctgaggcaggagaatcacttgaacccaggaggcggaggttgcagtgagccaagattgcgccattgcactccagcctgggcagcaagaacaaaactctgtctcaaaaaaaaaaaaaaaaaaaaaaaaaaaaaaaaaaaaaaggtaacatgccttgaccaggcatggtggctcatgcctgtaatcccagctctttgggaggctgaggcaagcggatcacgaggtcaggagatccaaaccatcctggctaacgcggtgaaaccccgtctctactaaaaatacaaaaaattagccaggcacggtggcacgcgcctgtagtcccagctactcgggaggctgaggcaggagtatcgcttgaaccccggaggcagaggttgcagtgagccgagatcacaccactgcactctagcctgggcgacagagtgagactccatctcaaaaaaaaaaaaaaaaaaaaagtaacatggatcaagatttggcaagaatgatttcatttagtccccagggagggcacatgcccccacttttcaagcggtaaatctgaggctttgaaaaggggcaggcgtggtgaggccacctagctgggtaggggatagaggcaagatttgaactctgatgtatgtaaccacagcctccgtgctgtctcctttttaacagcgacgttcacttttgagaataagaacagcagcctctgtctgtggatggtttgtgtgtcaggcagcctgctgagaaccttcacacacagcatcttatttagtgcggcaggaaccctttgagttagggtcagcggagatatttagaagcccagcacattgaaaaggatcctggaaactgccaacaccctcctctacccaaatatatattcaaactagaaaccccactaacatagatgaaaatccaagaatggcctaaatgttcaccctgtgataatccttttaatggattcttagaatcaagttatttaacaagcagatattgtcccgggggtagtgggtggcaggggagaaggagaagcttcactgatgacctgcattcttgcttaatggactctggaaatttagcattgcttcaaaatatagattatttatttccacttgacagaggaggatgctgaggctcaaaactgggaatgaacttgccccaaatcacacagctgagaagcggcctagctcttacttacattcagggctatgtagggacttcaataaaatccctaacaataaccacagtaatagtattgggagcatctaacgtggtaggcacagctatgttttgtacaccaattattttatttaggcctcctgggttcctattataactgagaggtcatgttctccattccacctggagctcagagaaggcataccatatggacactgacagaagctgccagtgaaggagctggggttcagggtggcccacaagtcctcaaataataacttgtgggagttggggggatgggaactatgggaggttggaagctcctgcctccttcatgttctgcccagatatcatttggtcagcgaggcccccctgccactccccctgcacacagttttgttgttgttgttgttgtttgagacagaatcttgctctgtcatccaggctggagtgcagtggcacaatcttggctcactgcaacctcggcctcctggattcaagcaattctcatgcctcagcctcctgagtagctgggattacaggcatgcaccaccatgtccggctaatttttgtatttttagtagagacagagttttgccatgttgcctagcctggtccggaactcctgagctcaaggcaatccgcccacctcggcctcccaaagtgctgggattacaggcatgagccaccgcacccagccaggaccaccgtatttaaaatttcaatcccccaacttctggtggtccccatccctgcctcattttttctccagagcacccattaccaaccatcaaactatatgttttatttatttaccatgtttacattctgtatccctccattaggaagtaaactccatgtgacaaagaggtttttttttttcatttgtttaatgctgggtccccacaccaagaacagtccctggcacacagcaggtgctcaatgattattggtacatagagtgaaagagatggagcctcaggctgacctagagagcaaggcaggaggaaaagataaaagggcccctcccctggggttttaggaccctcccaacgccccctaagccagtcttctctgcccccaggacccccggaacaaacacaagttccgcctgcatagctacagcagccccaccttctgcgaccactgtggctccctcctctacgggcttgtgcaccagggcatgaaatgctcctgtgagtgacctgggccttgccagggcccttccaaagcgcccggtctgggttccgggaaatgcccgggatggggtggggggtggagtcttggcttgggggcggggcctgaggtgctacccgcagctttcccctccaggctgcgagatgaacgtgcaccggcgctgtgtgcgtagcgtgccctccctgtgcggtgtggaccacaccgagcgccgcgggcgcctgcagctggagatccgggctcccacagcagatgagatccacgtaactggtgaggccccgccccctcgcctggccccgccccctccccaagtgtgaggcggggctgacccaaggcacttgtgctggcccagccctaccccaaagatggggccacgcctctttctatggtcacgcccacactcctgaccccaccccaaaggccgagcacacccagccataccccttttggctcgaagccccgcctccaacctggcttcttgcaactttctgcacctgttaatgactttgactttctttttttttttgggacggagtttcgctcttgttgctcaagctggagtgcaatggcgcgatctcggctcactgcaacttccgcctcccgggttcaagtgattctcctgcctcagcctcccgagtagctgggattacaggcgcgtgtcaccaagcccggctaattttttgtatttttagtacaaacggggtttcaccatgttagccaggctggtctcgaactcctgaccccaggtgatcccctcgactcggcctcccaaagtgctgggattaacaggcgtgagccaccgcgcctggccaatggctttcttttttgtttttattttatgtttatttttttgagatggagtcttgctctgtcacccaggctggagtgcagtggtgcaatcttggctcactgcaatctctgcctccggggttcaagggattctcctgcctcagcctcccgagtagctggaattacaggcgcctgccaccacatccggctaatttttttttttttttttttttgagacaagatctcgctctgttgcccaggctggagtgcagtagcatgatctcagctcactgcaacctccgcctctcaggttcaagcgattctcctgcttcagcctcctgagtagctgggactacaggtgcatgacactgcacccagctcatttttgtatttttagtagagacagggtttcaccatgctagccaggctggtctggaactcctgacctcaggtgatccgcccgcctccgcctcccaaagtgctgggattacaggggtgaggaccgtgcccggcaatggctttctgggtataaggatcttgagaagggagagtacctggttctgagggaggctgtggttcagtactggtgacatggccagggtccaaactctggttcctaatggagagaagggctctggatctgatttcagggtcactggttgcggaaagggctctatgccctgtcttctgggttctggagaggtaagaagtcatgagaaacgagactgagagcttggaattcttttttttttttttttgagacggagtctcgctgtgacgcccaggctggagtgcagtggcgtaatctcggctcactgcaagctccgactcctgggttcacgtcattctcccgcctcagcctcctgagtagctgggaccacagacacctgccaccacgcccagctaatttttttttttttttgtatttttagtggagacggggtttcaccattcacaggatggtctcgatctcctgaccttgtgatccgcccgccttggcctcccaaagtgctgggattacaggcatgagccgccgtgcctggccaagcttggaactcttgattgctgactggaggagggctgggagccccttcctggatctctaacccgtcacactcttcctcactccccgtttagttggcgaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttgtgagtctggggtgcagggaaggcaatgacagctgacagagaatgatctgagggtcctagtggcccccagagagcagctgatgggaggggttaggatagagggaacccagaaaagggcagaagaagatggtgggaaaagggaatagagtgattgagggagtgggatggagatacagaaacggagagacagccagaccactgtataattagtctccattgaagcccccaactttagagttagacagagatgagagagagaagagagagtctcagaagaggcagaaacccaaagagagacacagatggagagggaggggagaagatgggggatggcagggagacagagatcagttgacaggaagacagagtgatagagacccagagaggagagaagggtacagagactcagagagagagatctcgagagacaagagacagagatgggaaggggcggagaatgcaggaggaagggagaggaagagctctctaggtttacttcaggccccaaagccctagctggagagagagcccggctgggaaggtcagaggtcggagaccgacaaagcaggagaggagccccagctggctgggtttgcccccacctccagcaccaaggatggggaaccgaggggagccatgagctcggctctgcaccccatccaccccaccttcctgcagcaacctgaagccaggggatgtggagcgccggctcagcgtggaggtgtgggactgggaccggacctcccgcaacgacttcatgggggccatgtcctttggcgtctcggagctgctcaaggcgcccgtggatggctggtgaggagcagggctggggcctggggatggagcgcaatattaccatctccatctgtgtgtggtctctctcctccaggccactgtccttccctctgcctcccagcatgcgcacacacacacacacacacacacacacacgcacacacacgcacacacccctctctctctattcttctcttcttctcccctccctttctccctctccctctctttttatctcactctttctctcttccatctctgtgtccgtctctctgtgtctctttcctcccttccaatgtctttgcctctcccatgggtgccccatccccgctgcccgcctctggtctccgtctgtatgtcaggtacaagttactgaaccaggaggagggcgagtattacaatgtgccggtggccgatgctgacaactgcagcctcctccagaagtttgaggtacccagaccctggcttcctcaagggagcccagcccagcctcccacggttcagagctggcctttccttccacccctgagtgcccgctggtcctgggactacagttcccagaagaccctaggactccctcctctgctcttctaggggactcgagccccagggtctgatgggaattatagttcctatctatcgccatggcttgagggtactaggggccaccagcccctgttctagggcgatcccctgcatctcttgggaccctgactctctctttcttttctcccaggcttgtaactaccccctggaattgtatgaggtgagtagaaccagggcgttgaatggaggcagtttttgcctacttctctgatttcttattcctcctctgacttctgtcttcaattccccacacatgagttgagcacacatttgtgctaggcctgtcttgtgcttgctgaataatccaggatccagagatgaatctgaccctcaagcaactctccaaggtagggacacagtcacagatacttaaaatacaggaagatgtgctaaattagaggtagcccagggcactgaagaggcctaacggaggcactaatccagcctgggggagggtggtcagggaggacttccctgaggaggtgacgcctgaattgattcttgaggttttttaaaattttttaatttatttttatttttatttttatttttatttctgtcgcccaggctggagtgcaatggcacaatctcgcctcactgcaacctccagctcccgggttcaagcaattctcttgcctcagcctcctgagtagctgggattacaggtgaccgccaccacacccagctaattttttttttatttttagtagagatgggatttcgccatgttggccaggctgatctcaaactcccaaactcaggtgatccgcctgccttggcctcccaaagtgctgggattacaggcatgagccactgcgcccgaccgattcttgagttttttattttttttttgagacggagtctcgctgtgacgcccaggctggagtgcagtggtgcgatctcggctcactgcaagctccgcctcctgggttcacgccattctcctgcatcagcctcctgagtagctgggactacaggcgcccaccaccatgcccggctaattttttgtatttttagtagagatggggtttcaccgtgttagccaggatggtctcgatctcctgacctggtgatccacccgcctcagcctcccaaagtgctgggattacaggcgtgagccaccacacccagccgattcttgagttttaaaaaatctatcaagcatgatcatcttaatctctccattcattcattcactcactgaatatccttctttttctttctttctttctttctttttttttttgagacagaatctccttttgtcacccaggttggagtgcagtgatgcagtctcagctcactgcaacctctgcctcccagattcaagtgattctcctgcctcagcctcctgagtagttgggattacaggagcgcaccaccacacctggctaatttttgtatttttagtagagatggggtttcgacatgttggccaggctggtctcgaactcctgacctcaagtgatccacccgccttggcctcccaaagcgctgagattagaggcgtgagcgaccacgcccagacgaatacccattttctagggtgtcataagccaggccctgttctgggaatagaatcaggccattccctggtggagctcttcttctagtggaggacaaagttacaaacccagacattcacaacgaggagcaatgctgctgtaatggagacagcctcaggcactggggcgtccctggcacagcctgagtcagagaaagcttcctagagaggtgagacctggtagaagggcgggatttcccaaaggagagaccagattttcaggcaggaggaagtaatgctctctccctcatttaccctttcaaaaaatactttacagagcatctttgtgtgccaggcgtggctctactcactggggatatagagaaagcagggaaagaacaaacaaacaaacaaaaaagttcctttccttatgggatttacaccgggaggaagacattaaacaaaatatataagcatatgatagactgggcacgatgcctcatgtctgtgatcctagtaaggcgggcggatcacctgaggtcaggagtttgagaccagcctggccagcgtggcaaaaccccatctctactaaaaaatacaaaaatcagctgggcatggtggaggcgcctgtaatcccagctactcggaaggctgaggcaggagaattgctggattccgggaagtagaggctgcagtgagcccagatcgctccactgcactccagcctggatgacagagggagactctgtctcaaaaaaaaaaaaaaaaaaaaagaagacaagaatcttcaagattcaacaacagcaacaacatgttatagtctttactggactcttacagaaactttcaccagagtttttaatgttgtgtgtgggggttcacctgcatcagaattcctagagtgcttgcttttaaaagcacattccccagcctttctgcagacctactcagtgacgatctctctgatgcctcaaatgtctgcctactaaattaattcctcaggtgatccttttgcaaagttaagtttgagaatgggctctgcggccgggcgcagtggctcacgcctgtcatcccagcactttgggaggccaaggcgggtggatcacgaggtcaggagatcgagaccatcctggctaacacggtgaatccccgtctctactaaaaatacaaaaaaattagctgggcgtggtggtgggtgcctgtagtcccaggtactcaggaggctgaggcaggagaatggcatgaacctgggaggtggagcttgcaatgagccgagatcgtgccactgcactacagcctgggtgacagagcgagactctatctcagaaaaaaaaaaaaaagagagaatgggctctgcaggagacaagggtaccagcgggaggacattctgagccaaagaggtagagtcttttgagatcagcagggatgatcctcccgtacaaacccaagaaacccagcagggcagatggtgggcaaaggcctagaggcagggagtgtagggtggtgtgtgtgcctgttgtggctcacagcactctcccacagttcagcaggcaccacttaatattaccaatgaacaccaactctgtgccaagccttgagctaggtacggggctaacaacacagcaaacagaaacagccctgattattattattattattattattattattattattattattattatgtatttatctatttgagacacagtctcgctctgtcgcccaggctggaatgcagtggagcgatctcagctcactgcaacctctgcctcccgggttcaagcgattctcctgccttggcctcccaagtagctgggactacaggcatgtgccaccatgtcctactaatttttatattgctagtagagatggggcttcgccatgttggccaggctggtcttgaactcctgacctcaggtgatctgcccacctcggcctcccaaagtgctgggattacaggcatgagccaccgcacccagccctcaacaaatatttatgtagcctcaatgaggtaggcagtgttactgtgtcttagcgaacaaagcagacccctgccttagggagctcacaggcagaaagcagatagtcacacagatagatgtaaattactaagaataaaagtgccaggaaggtgctgtccatggtgaccaaggggtggtaagagaggcatctgacccagtttaaaaagtcaggcgaggcctctatgaagtgatgcttgagtcaaggtctaaagggtgtttgggagacaactaggagggaaggggaggggagagctttacaggaagacctaacggcacatccagaggccctgaggtgggagggaggacaatgagtgtcaggccagggtggctggaccatggagcctgggagagagaagaacaacctgcagtgtcagtctcagcctggctctgcaagtcatgtggaataaaatcttaacacagagggagcagttaaagggtttacaagcataggggagacatgacctggtttatttatttttaaattggctcctgtgcctgctgagtagagaatgcattagaaagggcagccgtccatgtagagggacaagtgtggaagctgtgacagcagcttagtcttgggccccctccctggggggccgaggcaggaaaaggtagagaagggaccctagctgaaagccaggtgtgctccctggactggcagcacccatgtcacccagaagctttttacacataacgattctcaggtcccaccccagatttatagagttagaaaatctggcagtgggacccagcaatctgttttaccaaaccctctagggaattccggcttagaggctaagagcaaccagattctagagctggactgcttgggtttcatttctggctctgtcctttacctgctgtgtgacttggggcaagttacttaacgtctctgtgctagtctcctcttctgtaaaatggaaacgatagcagggttttctggaaacagcatatgataagctatctaaaaaaaaaaaagaagaaaaaaagagctaagtgtttgttgaataataaataaaccctccaggctatggggagtcagagaaaattaagccaaggacagggtaggagggtggccattttcctctgtctagcgattctcatcctttcctttcttgggtgctgtgtctcttgggagcatttccttatcgctgtgtaaggtctaactgcctctggctctttctttctcctttccacagcgggtgcggatgggcccctcttcctctcccatcccctccccttcccctagtcccaccgaccccaagcgctgcttcttcggggcgagtccaggacgcctgcacatctccgacttcagcttcctcatggttctaggaaaaggcagttttgggaaggttggattcctggggttctgggggaaagggaggatgtctgtgggaaggtcagatttctggttcttagggaggaagtgggggtgggaagagactgggctcctgcatcttcaaatatggttaggttgggccgttcaggttcctggagaggagaggtttacagatgtggacactctccttgaggggacgggcggcaagtcagggctgtcagtcccttaagagatggaggaagggcctgggatcccgtttccctgcgtcccttagggagggggcaggtcctgtaccactgggttcccaacatggactggcccttttggaactgtgcgcataggtgatgctggccgagcgcaggggctctgatgagctctacgccatcaagatcttgaaaaaggacgtgatcgtccaggacgacgatgtggactgcacgctggtggagaaacgtgtgctggcgctggggggccggggtcctggcggccggccccacttcctcacccagctccactccaccttccagaccccggtaaggatggagggggcggaggctgtcctccgggccctgccttatccagttctggacatctgcgttgggattctgagtttagggcgaggcaagagaactttgtgctctctgagtgggcgaggccaggcggattgtctcctcagggggcgtggccgggggggggtccttggggggcgtggccaggcgaagggactcatcggggggcgtggccaggcggaggggctcaacggaggcgaggccgggtggaggggctcctcgggggcgtggccaggtggagggactcatcgggggcgtggccaggcagaggggctcttcgcggggcgtggtcaggcggatgaaatctttgggggggtggtttagaggggcgggctttgtcaggcgatgggatcattaataggcgtggccaggcagattggctccttgggggcgaggccaggcagacgagattatgaatgagcgtatccaggcaggtagattcttcggagggcgtggtcgggcggatgagctcctcgggggcgtggccaggcggtgagttcctcggtggcatggcctggccaggtgaatgggtcctgcggaggtgtcgtgaagcggttgagttccttgggggcgtggccaggtggatgggctcttggggggagtggccagatgcctgtttccctggggagcttggtcttgagtggctgtagccagtgctctggaattttcagcaaaggggcacagtggaggagggtgccttcctagtgggcctgcccagaattgggctccgagtgacggggtcatcacttttggattctgactgaaggacacatcagaaacaggacattatttccttaggattgcgacttaggggcagagagtcagaacctgcaagattttaagagggcgtgactttacttccaggggctccgaatgagagtggccagccacctggattaaaatatatgtatgagcaactttgattcctttttttttttttgagaaggagttagctcttgtcccccaggctggagtgcaatggcgcgatctcggctcactgcaacctccgcctcccgggtttaagcaattctcccgtctcagcctcctgagtagctgggattacaggctcccgccaccacactcagctgatttttgtatttttagtagagaccgggtttcgccacgttggccaggctggtctggaactcctgacctcaggtgatccacccgcttcggcctcccaaagtgctgggattacaggcgtgagccaccacgcccagctgcaactttgattcttagtaggaagccagaattgcatctgtgtgtgagtggctgtggaaagagattttggtgttcccggatttcgagcgaatggtgggcttcagtcttcaattctgagaaggcggggccagaacacgtggtctgatagttggcggtggtctggcgggtggagattctgaggtagcaggattagcaccttagggccctcccagggatgtggctaggtgctctgaatttctggttgggtgcatctggaaccttccacgtctgtcctgagtgatcaggaaagaaattctcctactctgggtagatggatcccgcctctaagcccatgcacttctccgcaggaccgcctgtatttcgtgatggagtacgtcaccgggggagacttgatgtaccacattcaacagctgggcaagtttaaggagccccatgcagcgtgagtctcggccaacagagaatggtcggggtggtggaagggggcaggatccagccactgaccttctgacgtccccacccaccccgtcctccaggttctacgcggcagaaatcgctatcggcctcttcttccttcacaatcagggcatcatctacaggtgagcagccccaggaatttccgtggaggaaatcacgcccctggaagggaagggatttgaatatgtggctctagactgctgaactcaacacttcttgcaattcctgccccacacccctgcatcgtccagggacctgaagctggacaatgtgatgctggatgctgagggacacatcaagatcactgactttggcatgtgtaaggagaacgtcttccccgggacgacaacccgcaccttctgcgggaccccggactacatagccccggaggtaaccccaaccctgctgctctggtcacgctttgagatcccttagagggtgtagctgatggtccagtattcaccacgggtgaggcctgaccctcagaccttgtcatgagttgtggccttcttacacagccagtcgttcctccagcctccagcacaggtgagcttggcactgagcctgccaggtgggcccagctgggtctctaaataggtaaggtgggcagcacctgtgggtgaatgttccaggagagtgggaccagctcgtaggaattccaagtaggacctgaccctggatccttctgagaaggggcagacgatttctagtgtactctgagtgggtgtggcctgtcccctgccaacactgaacatgtccggactatcttctgaatactttaaactgggcagggctctccctggagtattcagttggatggaagcttatttcctgtgttgtacgtgtttctctgatgtaagtgtactggacttctgtgctgcatttttcaagagggcaggatcagctgggcgcggtggctcacacctgtaatcccagcactttgggaggctgaggcaggtggatcacttgaggtcaggagtttgagaccagcctggccaacatggtgaaacctcatctctaacaaaattacacaaattagccgggcgtggtggcatgcgcctgtaatcccagctattcgggaggctgaggcaggagaatcgcttgaaccggggaggcggaggttgcagtgagctgagatcacaccactgcactccagcctgggtgacagagcataacttcataacttcatctcaaaaaaaaaaaaaaaaaaaagccgggtgcagtggctcacacctgtaattccagcacttgggaggctgaggcgggcggatcacaaggtcaggagtttgagaccagcctgactaacatggtgaaactccatctctactaaaaatacaaaaattagccaggcgtggtggcgggtgcctgtagtcccagctacttgggaggctgaggcaggagaattacttgaacccgggaggtggaggttgcagtgagctgagatcgcgccactgcactccagtctgggcaacagagtgagaccctgtctcaaaaaaaaaaaaaaaaagaaaaagaaaaagggcagggtgagatccctaaggttctgggagagcagatgctgtcctatgagtattttaagtgggtggggtattacccgactttgttaaaggggtggggctgatgttctgaatgtacgtatagatggataaagcacatgcctgtagtcccagctacttgggaggatgtgcctgactcgtgcccaaataatcaatgtcagtgatcacaaaacctggctggtaatcagaatcatctgtagaaaatttgaaaactgaggccagacatggtggctcatgcctgtaatcccagcactttgggaagctgaggcaggcagatcacttgaggtcaggaattcaagaccagcctagccaacatggtgaaaccccgtctctactaaaaatacaaaaattagctggacatggtgatgtgtgcctgtcaacccagctactcaggaggctgaggcaggagaatcacttgaacccaggaggtggaggttgcagtgagccaagattgcaccactgcactgcatcctgggctacagagtgagactccatctcaaaaaagaaaaaagataagaaaatttgaaaactacacacatattcctgactctgacacaaatattctaggtgggtggaaccagtgacttggccgttaggtagtctttctatttgaggccaaatgaatgtttgaagtaggtatgctttgtttccagaatattccaaaagttagatcgctggccaaaatattcagagtggaggctgggcgtggtggctcactcctataatcccagcactttgggaggcggaggcaggccaattgcttgagtccgggagtttgagaccagcctgggcaacatagtgaggacctatcttcactaaaagtgcaaaaattagccaggtgtggtggtgcacacctgtagcccacctacttgggaggctgaggtagaagaattacctgagcctgggaagttgaggctgagtgagccgtgatcacactactgtgctccagcctgggcaacagagtgagaccctgtcaaaaaaaaaaaaaaaaaaaaaaaaaacgaaacaaaaaatcacctgatgaaataaatattcagagtgggaagagcttgtgctgaaagcacttaacgtgggtagcgctcccagggggtgaggccagaggggtcctaggcttcctaaagaacgcatcatgattccctgccttccacctcccctagatcattgcctaccagccctatgggaagtctgtcgattggtggtcctttggagttctgctgtatgagatgttggcaggacaggtaagggaaggtggggagaagctggcttggctaaaagagacagagaggggcacctggatctcaggaggagccagttagaaaggagcccagaaggttgtgctcgaatagcgctgtccatggttctgaagtgttgtcttaatgtagaccaggtgttttgttttgttttgttttgtttgttctgttaccatggattctttctccctagatggtaaagtagcagtctggttaagcctatggatcccttctcagaagaatgtttttcaatgcacaaaatagaataatacatacgcagaaaaccaaggttcaaatcctcactttgccacttactggctgtgtcagcttagacaattacatacattctaaatcagcttgattggattcctggactgctgagtgtactacaactaaaaaaattggttaccacactcttgatcatcttcttagctggctatggtagtgggcacctatagtcccagctactggggaggctgaggcaggagggtcacatgagcccaagaactcaaggttacactgaactatgagtatgccactgtgttccagcacaggcgacagagcaagaccccatctcaaaagaacaaacaaacaaaagaaatcaatcattttgaaagatagtcaccaaaacttttaaaatttaattgaatctaacaaccaactaaattcaatgtatacagttgtctaaatatttaaaattggaaaatggacatatgtgcaatttcttattaattcattaagtagtaagttctaatggtagtaaagtaaaaaggagctctaacagtaattttgatgtagcaatgactaataatttggaatatctgcaactctcatgtgatgagaaaatacctgtgattactattgataatgaagccacaagcactgctgctgcctgcattcataactgaagggaatgctaaattttagttagaagttaaaaaaaaatttggccggacatggtggctcacgcctgtaatccaagcactttgggaggccgaagtgggcagatcacttgaggtcaggagtttgagaccagcctggccaatatgatgaaaccccatctctaaatctttgccttggtatcattttttgtaacctcagaagactgtgaactactcatccaaccaggagaatgcttttagggtgtttcctgcagtttttcctctcttctatttaactgacatgttgcataattaacagcctgctgatttacatagcagataaagagaggcagaatagtacagagatgcacagatctgaggcatccgagataggaaatgagagaacctgagaaggagagagatcaagctttggtggtttggtctgatctctcctgagggtgtggtcaggtgtgcatgtggggcgtgtgatgggtcaggcatgttcccggtggggtgaggagggtgtggaaggtttggggaaaggcagttgggcatgtccctgactctctatcccctccactttgatagcctcccttcgatggggaggacgaggaggagctgtttcaggccatcatggaacaaactgtcacctaccccaagtcgctttcccgggaagccgtggccatctgcaagggggtgagagccccctgactcccagcttctccaggctcacaaccacacaccccattgctgtctctgtgcctattagaaaaatgctcccattcctgaagtcactttacttccatctgttggaaaagttgatatgatgcataggttttgttagaacaatgatttccagccctgttgccacgaggcctggagatggcctctgtctcatccttctctgtgactcccactccccagctccctgcttgcaggaagtgctgaaagtccagggtgtctgtctgtctagaactgggtgggtcaggtaaacccaacttctgcagcttttcttcctgtgtgaacttgggtgagtcaccaaaactttgtgagcttaactctcttcaggggttatggagttgacacagaagaaagcacctggcccatagcagattttcagcccatgtcagcaccttctgcgtctagctgctctctctgcatctccctgacagtctctctggtttctgtctcatgcctcccctccatctgcatacgatggggctctctgtgtttcttccttttctctgtgtctccttctgcatctctgtctacacttttgggcctttgtccaacccccctaccccccatctctgtcccctcctgtgtccactctcatacacacacgctacatctcaccctcctctcctgctggctttctgtcctcccttttctctgggtctctgtccccatatttggtctttattcctccctctgggtgtgtgtttcctgggtctctatccttctctctttctgaatctctgtccccctgggtctcctctgtctcctcccttctctgagtttctgccttcttacctggggtctctgtaccaccctctgaatttctatttccccttttctctgggtctgcaacctctcttcccccattccctccctcttccccttcatctcctctacccttccctccctccccctaccgacctccctctgtccttcactctccccttccctccctccccctacccttccctctccccgtccctctctccccttccctctctccccctaccccttttccctctcctcttctccatctccctcatgatttgttctgtttctctgtgtgtccctgggtctctgggtatgaatttcatctgctatcattttcatgtatcctctccttcttcctgtctctctctctctctctctcactcactctcaccctctgtctctctctctctctgtccctcttcctctctctctcctctctgtctctctctgtctcgctctctgtctgtctccctcctcctcctcctccctcctcctcctcctcctcctcctcctcctcctcctcctcctccttcttcttcttcttcttcttcttcttcttcttcttttcttctctctctctctcctttcttttcccttcctccctccatttgcctgtttcccctggctgttcttatctctccggatctcatgcctgtgtctcttggttcctccatctgcctgtctctgtccctctttctctgggtctacctgtccggcactctgtctgtttgtctgtctgtctctctctgtgtttcccacagttcctgaccaagcacccagggaagcgcctgggctcagggcctgatggggaacctaccatccgtgcacatggctttttccgctggattgactgggagcggctggaacgattggagatcccgcctcctttcagaccccgcccggtcagtcaccctccaggcaacaaaaacctggtccctgaaggggtggggttcccctgggcctcaatatacctgtatgtgggggtggggttccctctgcagagccccccgcccccaacaaaaggaggtgcagacaccatgaagcatgaatagagattctgcaggagacaggagatgagactggggtacacagagggacacccgaggagccctcggagctgcttaactttccctcccccacgtctcccacagtgtggccgcagcggcgagaactttgacaagttcttcacgcgggcggcgccagcgctgacccctccagaccgcctagtcctggccagcatcgaccaggccgatttccagggcttcacctacgtgaaccccgacttcgtgcacccggatgcccgcagccccaccagcccagtgcctgtgcccgtcatgtaatctcacccgccgccactaggtgtccccaacgtcccctccgccgtgccggcggcagccccacttcacccccaacttcaccaccccctgtcccattctagatcctgcaccccagcattccagctctgcccccgcgggttctagacgcccctcccaagcgttcctggccttctgaactccatacagcctctacagccgtcccgcgttcaagacttgagcg

SEQ ID NO: 19 - DNA, 2524 bp, Homo sapiens
  misc_feature: PKCG according to GB Acc. XM_017991
ggcgcgcccggggtgccgctccctgcctggcgcgctccgcacctggaggtgccttgcccctctcctgcccacctcggaatttccctgtggctcctttgatccttcgagtctccagctcctctcccttccacctgtttcccccaagaaaggcaggatcctggtccctgctacgtttctggggccatggctggtctgggccccggcgtaggcgattcagaggggggaccccggcccctgttttgcagaaagggggccctgaggcagaaggtggtccacgaagtcaagagccacaagttcaccgctcgcttcttcaagcagcccaccttctgcagccactgcaccgacttcatctggtgagggaagggggcagggcctgcaatgtcaaggtaagagctggggtggttcatcgacgatgccacgaatttgtgaccttcgagtgtccaggcgctgggaagggcccccagacggacgtgagtgctcggaccggaacaaacacaagttccgcctgcatagctacagcagccccaccttctgcgaccactgtggctccctcctctacgggcttgtgcaccagggcatgaaatgctcctgtgagtgagatgaacgtgcaccggcgctgtgtgcgtagcgtgccctccctgtgcggtgtggaccacaccgagcgccgcgggcgcctgcagctggagatccgggctcccacagcagatgagatccacgtaactggtgagggaggcccgtaacctaattcctatggaccccaatggtctctctgatccctatgtgaaactgaagctcatcccagaccctcggaacctgacgaaacagaagacccgaacggtgaaagccacgctaaaccctgtgtggaatgagacctttgtgttgtgagtcctgaagccaggggatgtggagcgccggctcagcgtggaggtgtgggactgggaccggacctcccgcaacgacttcatgggggccatgtcctttggcgtctcggagctgctcaaggcgcccgtggatggctggtgacaagttactgaaccaggaggagggcgagtattacaatgtgccggtggccgatgctgacaactgcagcctcctccagaagtttgaggtactgtaactaccccctggaattgtatgaggtgagcgggtgcggatgggcccctcttcctctcccatcccctccccttcccctagtcccaccgaccccaagcgctgcttcttcggggcgagtccaggacgcctgcacatctccgacttcagcttcctcatggttctaggaaaaggcagttttgggaagggtgatgctggccgagcgcaggggctctgatgagctctacgccatcaagatcttgaaaaaggacgtgatcgtccaggacgacgatgtggactgcacgctggtggagaaacgtgtgctggcgctggggggccggggtcctggcggccggccccacttcctcacccagctccactccaccttccagaccccggaccgcctgtatttcgtgatggagtacgtcaccgggggagacttgatgtaccacattcaacagctgggcaagtttaaggagccccatgcagcggttctacgcggcagaaatcgctatcggcctcttcttccttcacaatcagggcatcatctacaggggacctgaagctggacaatgtgatgctggatgctgagggacacatcaagatcactgactttggcatgtgtaaggagaacgtcttccccgggacgacaacccgcaccttctgcgggaccccggactacatagccccggaggatcattgcctaccagccctatgggaagtctgtcgattggtggtcctttggagttctgctgtatgagatgttggcaggacaggcctcccttcgatggggaggacgaggaggagctgtttcaggccatcatggaacaaactgtcacctaccccaagtcgctttcccgggaagccgtggccatctgcaaggggggttcctgaccaagcacccagggaagcgcctgggctcagggcctgatggggaacctaccatccgtgcacatggctttttccgctggattgactgggagcggctggaacgattggagatcccgcctcctttcagaccccgcccggtgtggccgcagcggcgagaactttgacaagttcttcacgcgggcggcgccagcgctgacccctccagaccgcctagtcctggccagcatcgaccaggccgatttccagggcttcacctacgtgaaccccgacttcgtgcacccggatgcccgcagccccaccagcccagtgcctgtgcccgtcatgtaatctcacccgccgccactaggtgtccccaacgtcccctccgccgtgccggcggcagccccacttcacccccaacttcaccaccccctgtcccattctagatcctgcaccccagcattccagctctgcccccgcgggttctagacgcccctcccaagcgttcctggccttctgaactccatacagcctctacagccgtcccgcgttcaagacttgagcg

SEQ ID NO: 20 - DNA, 29 bp, Artificial
  Forwards primer Accession number RSTS1G64894
tcctggctcataattccataacccttggt

SEQ ID NO: 21 - DNA, 28 bp, Artificial
  Reverse primer, Accession Number RSTS1G64894
gtgcgccaccttttcagagttcttttgt

SEQ ID NO: 22 - DNA, 30 bp, Artificial
  Forwards primer, Accession Number RSTS2G64900
ttatgcctgattcagtgacactactttttc

SEQ ID NO: 23 - DNA, 27 bp, Artificial
  Reverse primer, Accession number RSTS2G64900
cggctgcctctaacatacggaagattc

SEQ ID NO: 24 - DNA, 25 bp, Artificial
  Forwards primer, Accession Number RSTS3G64895
gaggctgacgcggcggctctatctc

SEQ ID NO: 25 - DNA, 25 bp, Artificial
  Reverse primer, Accession Number RSTS3G64895
agcggttgcgtcaccggtgcagaag

SEQ ID NO: 26 - DNA, 26 bp, Artificial
  Forwards primer, Accession Number RSTS4AF260531
atatacgtgtatcagtttcagaatgc

SEQ ID NO: 27 - DNA, 26 bp, Artificial
  Reverse primer, Accession Number RSTS4AF260531
tattttaaagtcagacatgaaaaagg

SEQ ID NO: 28 - DNA, 27 bp, Artificial
  Forwards primer, Accession Number RSTS5AF260970
gggccaggaaaatgcacggaagtgaag

SEQ ID NO: 29 - DNA, 27 bp, Artificial
  Reverse primer, Accession Number RSTS5AF260970
cccgggcttgtggaattaactgagcag

SEQ ID NO: 30 - DNA, 31 bp, Artificial
  Forwards primer, Accession Number RSTS6G64896
ttatgtgggccccaaaccattactgatactc

SEQ ID NO: 31 - DNA, 30 bp, Artificial
  Reverse primer, Accession Number RSTS6G64896
ctcatgcccataaacccctaaattccttgt

SEQ ID NO: 32 - DNA, 28 bp, Artificial
  Forwards primer, AccessionNumber RSTS7G64897
gcctgctaagcctctgtgcctagtaaag

SEQ ID NO: 33 - DNA, 30 bp, Artificial
  Reverse primer, Accession Number RSTS7G64897
ggtatcagaaaggtacaatccctatgtctc

SEQ ID NO: 34 - DNA, 29 bp, Artificial
  Forwards primer, Accession Number RSTS8G64901
cccaaaagatcccaagtccaggcagaaag

SEQ ID NO: 35 - DNA, 27 bp, Artificial
  Reverse primer, Accession Number RSTS8G64901
cctgggaagctcacaagggtggaagac

SEQ ID NO: 36 - DNA, 25 bp, Artificial
  Forwards primer, Accession Number RSTS9G64898
gtacattggcatccgcaggggtaac

SEQ ID NO: 37 - DNA, 27 bp, Artificial
  Reverse primer, Accession Number RSTS9G64898
cctctcaaggcgtgctttttcgtaaga

SEQ ID NO: 38 - DNA, 19 bp, Artificial
  Forwards primer, Accession Number RSTS10 G64899
agcccgctcgtatctatgg

SEQ ID NO: 39 - DNA, 21 bp, Artificial
  Reverse primer, Accession Number RSTS10 G64899
tgaggactacttgggcacagg

SEQ ID NO: 40 - DNA, 24 bp, Artificial
  HPKCGup1 Primer
ccttgcccctctcctgcccacctc

SEQ ID NO: 41 - DNA, 34 bp, Artificial
  HPKCGlo2 Primer
gggacggctgtagaggctgtatggagttcagaag

SEQ ID NO: 42 - DNA, 19 bp, Artificial
  GAPDH2 Primer
gtgaaggtcggagtcaacg

SEQ ID NO: 43 - DNA, 22 bp, Artificial
  GAPDH3 primer
ggtgaagacgcccagtggactc